(19) United States
(12) Patent Application Publication
(10) Pub. No.: US 2007/0128129 A1
(43) Pub. Date: Jun. 7, 2007

(54) ENZYMATIC BLEACHING SYSTEM

(76) Inventors: Regina STEHR, Neuss (DE); Cornelius BESSLER, Dusseldorf (DE); Karl-Heinz MAURER, Erkrath (DE); Susanne WIELAND, Dormagen-Zons (DE); Nina HOVEN, Dusseldorf (DE); Inken PRUSER, Dusseldorf (DE)

Correspondence Address: PAUL AND PAUL, 2000 MARKET STREET, SUITE 2900, PHILADELPHIA, PA, 19103, US

(21) Appl. No.: 11/610,958

(22) Filed: Dec. 14, 2006

Related U.S. Application Data
(63) Continuation of application No. PCT/EP05/06178, filed on Jun. 9, 2005.

(30) Foreign Application Priority Data
Jun. 18, 2004 (DE) 10 2004 029475.5

(51) Int. Cl. A61K 8/96; C11D 3/386; C12N 9/02

(52) U.S. Cl. 424/50; 510/320; 424/62; 435/189

(57) ABSTRACT

The present invention relates to a novel enzymatic bleaching system, including at least one oxidase and at least one perhydrolase, body care agents, hair shampoos, hair care agents, mouth-, tooth- or denture care products, cosmetics, therapeutics, textile detergents, cleaning compositions, rinsing agents, textile detergents for automatic washing machines, hand detergents, hand dishwashing agents, automatic dishwasher agents, disinfectants and agents for bleaching or disinfecting filter media, textiles, hides, paper, skins or leather, comprising the novel enzymatic bleaching system, as well as uses of the novel enzymatic bleaching system and this composition.

### CROSS REFERENCE TO RELATED APPLICATIONS

The present application is a Section 365(c) continuation of International Application No. PCT/EP2005/006178 filed 9 Jun. 2005, which in turn claims the priority of DE Application 10 2004 029 475.5 filed Jun. 18, 2004, each of which is incorporated herein by reference.

### FIELD OF THE INVENTION

The present invention relates to a novel enzymatic bleaching system, including at least one oxidase and at least one perhydrolase, body care agents, hair shampoos, hair care agents, mouth-, tooth- or denture care products, cosmetics, therapeutics, textile detergents, cleaning compositions, rinsing agents, textile detergents for automatic washing machines, hand detergents, hand dishwashing agents, automatic dishwasher agents, disinfectants and agents for bleaching or disinfecting filter media, textiles, hides, paper, skins or leather, comprising the novel enzymatic bleaching system, as well as uses of the novel enzymatic bleaching system and this composition.

### BACKGROUND OF THE INVENTION

Usually, inorganic, highly alkaline hydrogen peroxide suppliers, such as percarbonate or perborate in combination with bleach boosters (TAED or NOBS), are employed for the conventional chemical bleaching of washing. However, this standard bleaching system is fully effective only at temperatures above 60° C. This type of bleaching system is not well suited for the low temperature range. Moreover, local spotting frequently occurs with colored textiles. The bleach component hydrogen peroxide is formed by spontaneous decomposition of the salt and thereby rapidly, but for a short period, yields high concentrations. A gentle, delayed bleaching at milder pH is consequently not usually possible.

In addition, enzymatic systems have also been developed, for example those according to EP 1002041 B1, based on non-heme containing haloperoxidases. A further very complex system emerges from e.g. DE 10126988 A1: This comprises an enzymatic system for manufacturing reactive oxygen species as the “system component A” and as the “system component B” an organic compound described as the “precursor”, which is activated by the enzymatic system and performs the actual bleaching reaction.

For hair dyeing, bleaching is usually carried out with hydrogen peroxide and ammonia solution before dyeing in order to level the gray tones. The short-term high concentration of hydrogen peroxide in combination with the alkaline pH lead to significant damage to the hair.

Enzymes often exhibit secondary activities besides their true, i.e. best catalyzed reactions. Thus, in a secondary reaction, many proteases can liberate percarboxylic acids as the bleaching agent from carboxylic acid esters with the help of hydrogen peroxide.
CH₃CH₂CH₂COOCH₃+H₂O₂→CH₃CH₂CH₂COOOH+CH₃OH

Up to now, this perhydrolase-secondary reaction of proteases was not sufficient for an economic use of proteases as the bleaching enzyme in detergents and cleaning compositions and was therefore not further considered for technical purposes.

Proteases and of these, the subtilisins in particular, have long been used as wash-active substances in detergents and cleaning compositions, simply because of their proteolytic activity. Also included is the protease subtilisin Carlsberg, which is presented in the publications of E. L. Smith et al. (1968) in J. Biol. Chem., volume 243, p. 2184-2191, and of Jacobs et al. (1985) in Nucl. Acids Res., volume 13, p. 8913-8926. It is formed naturally from Bacillus licheniformis and was or is obtainable under the trade name Maxatase® from Genencor International Inc., Rochester, N.Y., USA, as well as under the trade name Alcalase® from Novozymes A/S, Bagsværd, Denmark.

Variants of this enzyme obtained by point mutations have also been described, which in relation to their use in detergents and cleaning compositions, however were solely orientated to an optimization of the proteolytic activity. Thus Carlsberg variants with reduced binding to the substrate with simultaneously increased hydrolysis rates are known for example from the application WO 96/28566 A2.

For example, WO 95/10591 A1 discloses a plurality of possible point mutations on subtilisins, which are each intended to be combined with an amino acid exchange in position 76 in the count of subtilisin from B. licheniformis (BPN′). These are intended to increase the performance attributed to the protease activity of these enzymes, especially in detergents and cleaning compositions. That other mutations could cause a shift in activity of such enzymes away from proteolysis and towards the above described perhydrolase secondary activity and that these variants, due to their shift in activity, could be of interest for use in detergents and cleaning compositions neither emerges from this document nor from others that describe other point mutations of subtilisins.

The not previously published application WO 2004/058961 A1, which is based on the application DE 10260903.9 from the applicant, describes perhydrolases, obtained from the protease subtilisin Carlsberg by means of point mutagenesis, which can be employed in bleaching systems and which extensively avoid the disadvantages associated with the relevant prior art.

Oxidases, such as for example alcohol oxidases or amino acid oxidases, are known from the prior art. It is also known that oxidases together with their substrates can be employed for hydrogen peroxide generation for bleaching and inhibiting color transfer in detergents or for enzymatic hair dyeing and bleaching in cosmetic compositions.

In WO 97/21796 A1, it is described that oxidases, with the help of atmospheric oxygen, liberate hydrogen peroxide from their corresponding substrates under technical conditions (for example from a detergent matrix). Hydrogen peroxide is continuously formed, the efficiency of the product formation being dependent on the temperature stability and pH stability, as well as the tolerance against substrate and product.

Choline oxidases are also known per se from the prior art, including the following cited publications: Ikuta, S., Imamura, S., Misaki, H., und Horiuti, Y. (1977): “Purification and characterization of choline oxidase from Arthrobacter globiformis”; J. Biochem. (Tokyo), volume 82, pages 1741-1749 and Deshnium, P., Los, D. A., Hayashi, H., Mustardy, L., und Murata, N. (1995): “Transformation of Synechococcus with a gene for choline oxidase enhances tolerance to salt stress”; Plant Mol. Biol., volume 29, pages 897-907. Choline oxidase from A. globiformis is also listed under the numbers AAP68832 and AAS99880 in the databank GenBank (National Center for Biotechnology Information NCBI, National Institutes of Health, Bethesda, Md, USA.

The not previously published application WO 2004/058955 A2, which is based on the application DE 10260930.6 from the applicant, describes how choline oxidases can be isolated from the bacteria Arthrobacter nicotianiae and A. aurescens and further developed, and can be employed in bleaching systems which extensively avoid the disadvantages associated with the relevant prior art.

Enzymatic bleaching systems that comprise a combination of oxidases and perhydrolases have not been described up to now.

### SUMMARY OF THE INVENTION

Accordingly, the object was to provide a novel bleaching system that is suitable for technical purposes. If possible, this should afford better bleaching performance, especially when employed in detergents or cleaning compositions. Additional advantages were seen in a continuous as possible and therefore comparatively gentle reaction.

Surprisingly, it was found that an enzymatic bleaching system that comprises a combination of oxidases and perhydrolases not only extensively avoids the disadvantages in bleaching systems associated with the relevant prior art, but also affords a significantly better bleach performance than the use of oxidases or perhydrolases alone.

The subject matter of the present invention is accordingly an enzymatic bleaching system comprising at least one oxidase and at least one perhydrolase.

The elegance of this system consists in that both system components engage with each other over the provisionally formed hydrogen peroxide, thus effecting the expected gentle reaction; as excessively high bleaching agent concentrations do not occur (in general initially in the prior art), instead the bleaching agent is formed comparatively continuously. Moreover, as supported by the examples of the present application, this system has proven to perform better than a system solely based on oxidase.

In accordance with the above stated reasons, oxidases are understood to mean those enzymes that oxidize their specific substrate with the help of atmospheric oxygen, thereby releasing hydrogen peroxide. Consequently, the oxidase is advantageously present together with its specific substrate in the inventive enzymatic bleaching system.

In a more specific embodiment of this, both are brought into contact with each other at the moment of the intended reaction, for example, both components are indeed present next to one another but are separated by a barrier that is removed only at a given time. This can happen, for example, by encapsulating the enzyme and/or the substrate, wherein these capsules dissolve away, for example, on contacting the bleaching system with water and the components only then react with one another.

The inventively employable oxidase is preferably selected from 2 electron oxidoreductases in combination with their specific substrates, for example
Pyranose-oxidase (E.C. 1.1.3.10) and for example D-glucose or galactose,
Glucose-oxidase (E.C. 1.1.3.4) and D-glucose,
Glycerine-oxidase (E.C. 1.1.3.21) and glycerine,
Pyruvate-oxidase (E.C. 1.2.3.3 or E.C. 1.2.3.6) and pyruvic acid or its salts,
Alcohol-oxidase (E.C. 1.1.1.1) and alcohol (MeOH, EtOH),
Lactate-oxidase (E.C. 1.13.12.4) and lactic acid or its salts,
Tyrosinase-oxidase (E.C. 1.10.3.1 or E.C. 1.14.18.1) and tyrosine,
Uricase (E.C. 1.7.3.3) and uric acid or its salts,
Amino acid-oxidase and each of its oxidizable amino acids, including particularly
Choline oxidase (E.C. 1.1.3.17 or E.C. 1.1.99.1) and choline.

According to the invention, peroxidases (E.C. 1.11.1.7.), due to their catalysis of reactions that consume hydrogen peroxide, are not considered as oxidases.

In accordance with the above statements, perhydrolases are understood to mean those enzymes that with the help of hydrogen peroxide—that is advantageously prepared by the oxidase reaction—release percarboxylic acids as the bleaching agent from carboxylic acid esters. Accordingly, in inventive enzymatic bleaching systems, in addition to the perhydrolase, a carboxylic acid ester or another carboxylic acid derivative is advantageously made available, which reacts under perhydrolysis conditions to afford the corresponding percarboxylic acid.

It is possible, although generally not absolutely necessary, to provide both these components separately from one another because the agent that triggers the reaction, namely hydrogen peroxide (when not other possibly present bleaching systems), is only made available by the oxidase reaction.

In a preferred embodiment, the inventive bleaching system is characterized in that the oxidase is selected from:
a) choline oxidases, whose amino acid sequence matches that listed in SEQ ID No. 2 to at least 76.5%, increasingly preferably to at least 78%, 80%, 82%, 84%, 86%, 88%, 90%, 92%, 94%, 95%, 96%, 97%, 98%, 99% and particularly preferably to 100%,
b) choline oxidases, whose amino acid sequence matches that listed in SEQ ID No. 4 to at least 89%, increasingly preferably to at least 90%, 92%, 94%, 95%, 96%, 97%, 98%, 99% and particularly preferably to 100%,
c) choline oxidases, whose amino acid sequence matches that listed in SEQ ID No. 6 to at least 83.8%, increasingly preferably to at least 84%, 86%, 88%, 90%, 92%, 94%, 95%, 96%, 97%, 98%, 99% and particularly preferably to 100%,
d) choline oxidases, whose amino acid sequence matches that listed in SEQ ID No. 28 to at least 76.4%, increasingly preferably to at least 78%, 80%, 82%, 84%, 86%, 88%, 90%, 92%, 94%, 95%, 96%, 97%, 98%, 99% and particularly preferably to 100%, and
e) choline oxidases according to a), b), c) or d), which are obtained by one or multiple conservative amino acid exchanges from a choline oxidase according to a) to d) or by derivatization, fragmentation, deletion mutation or insertion mutation of a choline oxidase according to a) to d).

Therefore, those choline oxidases are preferably used that are described in the previously unpublished application WO 2004/058955 A1 that is based on the application DE 10260930.6 (see above) whose disclosure is incorporated herein by reference in its entirety. It describes—including a corresponding homology region—the following choline oxidases that were first isolated by the applicant and which are also reported in the sequence listing of the present application both with their nucleotide sequence (each odd numbered) and also with their amino acid sequence (each even numbered).
a) the choline oxidase with the designation KC2 from Arthrobacter nicotiniae, of which a strain is deposited in the Deutschen Sammlung von Mikroorganismen und Zellkulturen GmbH, Mascheroder Weg 1b, 38124 Braunschweig (http://www.dsmz.de) under the number 96-878 (therefore the name “DSMZ-ID 96-878”), listed here under SEQ ID NO. 1 and 2;
b) the choline oxidase from Arthrobacter aurescens, of which a strain is deposited in the American Type Culture Collection, 10801 University Boulevard, Manassas, Va. 20110-2209, USA (http://www.atcc.org) under the number ATCC 13344 (therefore the name “ATCC 13344”), listed here under SEQ ID NO. 3 and 4;
c) a hybrid choline oxidase from both of the previous choline oxidases from A. nicotianiae and A. aurescens, which is listed here under SEQ ID NO. 5 and 6; and
d) an N-terminated deleted choline oxidase (with the designation KC2s) that derives from the cited choline oxidase from A. nicotianiae and listed here under SEQ ID NO. 27 and 28; therefore the sequence listing comprises the following information: “N-terminal deleted choline oxidase from Arthrobacter nicotianiae (DSMZ-ID 96-878)”; in addition it should be noted that by deleting the first amino acid, the codon “ttg” coding for leucin usually appears as the first codon, such that during expression in organisms, whose translation apparatus is set for that, this is translated as methionine; alternatively, it can also be artificially changed into “atg”, to obtain methionine as the first amino acid; therefore, the sequence listing additionally comprises the following information: “First codon will be translated as Met or can artificially be changed into atg, coding for Met.”

The establishment of the DNA sequence facilitates the reproducibility of the present invention for the case where the enzyme itself were not available and therefore one has to fall back on the associated gene that then has to be expressed using known molecular biological methods, as a result of which one obtains the derived protein.

As is explained in example 2 of the present application, the choline oxidase from A. globiformis, listed in the data bank of the NCBI under the numbers AAP68832 and AAS99880 exhibits the following homology values on the amino acid level to these choline oxidases over each total length: to the choline oxidase from Arthrobacter nicotianiae (KC2; SEQ ID NO. 2) 77.7% identity, to the choline oxidase from A. aurescens (SEQ ID NO. 4) 89.6% identity, to the hybrid choline oxidase according to SEQ ID NO. 6 84.5% identity and to the N-terminal deleted choline oxidase from A. nicotianiae (KC2s; SEQ ID NO. 28) 78.5% identity. Their combination with a perhydrolase to an inventive bleaching system is not previously described there nor suggested.

Fundamentally, according to the invention, the homology values concerning the molecules from the prior art are determined according to the method listed by D. J. Lipman and W. R. Pearson in Science, volume 227 (1985), p. 1435-1441, preferably with the computer program Vector NTI® Suite 7.0 with the set default parameters, which is available from the company InforMax Inc., Bethesda, USA.

Further, a no less preferred embodiment of the present invention is illustrated by the selection of a choline oxidase that corresponds to the previous definitions of inventive choline oxidases, including their respective homologous regions, and which are obtainable by a mono or polyconservative amino acid exchange from an inventively useable choline oxidase or by derivatization, fragmentation, deletion mutation or insertion mutation of an inventively useable choline oxidase.

In other words: the above described inventive choline oxidases can be further developed within the listed homology regions by the use of customary molecular biological processes. Such further developments can concern, for example, higher stability values, improved enzyme kinetic parameters, a higher product formation rate (especially for H₂O₂) or modifications of substrate specificity. They then characterize correspondingly preferred embodiments of the present invention.

In the presence of atmospheric oxygen, the inventively employable choline oxidases are able to continuously release hydrogen peroxide from choline and choline derivatives through the formation of betaine aldehyde and betaine.

The inventively employable choline oxidases advantageously exhibit a high specific rate of formation of hydrogen.

The pH profile of the inventively employable enzymes is preferably compatible with the pH needed for technical use, as well as with typical products such as detergents and cleaning compositions and hair dyes. Among these may be cited a desirable highest possible stability towards denaturing agents such as for example surfactants.

The addition of choline and its derivatives, together with the associated formation of the additional reaction product betaine or the corresponding derivatives, results in a considerable secondary benefit, because such compounds can be used as potential softeners.

Examples of substrates are choline and derivatives of N-substituted aminoethanol with the structural formulae 1 or 2:

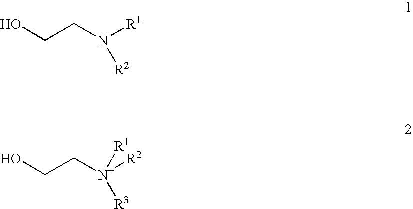

Where, for structural formula 1:
R¹=H, R²=2-hydroxyethyl
R¹=methyl, R²=methyl
R¹=2-hydroxyethyl, R²=2-hydroxyethyl

These compounds are known from the literature to be substrates for the choline oxidase from A. globiformis.

For structural formula 2:
R¹=R²=R³=methyl

A further inventively suitable substrate is betaine aldehyde (OHC—CH₂)N⁺(CH₃)₃.

The additional effect of the betaine being formed is of considerable interest for hair care.

In the context of the present application, a statement of the type “at least X %” means “X % to 100% (including the limiting values X and 100 and all integral and non-integral percentages in between).”

In a preferred embodiment, the inventive bleaching system is characterized in that the perhydrolase is selected from:
a) perhydrolases, whose amino acid sequence corresponds to that listed in SEQ ID NO. 26, but carries one or a plurality of exchanged amino acids at the sequence positions selected from 11, 15, 21, 38, 50, 54, 58, 77, 83, 89, 93, 96, 107, 117, 120, 134, 135, 136, 140, 147, 150, 154, 155, 160, 161, 171, 179, 180, 181, 194, 205, 208, 213, 216, 217, 238, 239, 251, 253, 257, 261,
b) perhydrolases, whose amino acid sequence corresponds to that listed in SEQ ID NO. 26, but carries one or a plurality of exchanged amino acids at the sequence positions selected from 11, 58, 77, 89, 96, 117, 120, 134, 135, 136, 140, 147, 150, 161, 208, 216, 217, 238,
c) perhydrolases, whose amino acid sequence corresponds to that listed in SEQ ID NO. 26, but carries one or a plurality of exchanged amino acids at the sequence positions selected from 58, 89, 96, 117, 216, 217,
d) perhydrolases, whose amino acid sequence corresponds to that listed in SEQ ID NO. 26, but exhibits one or a plurality of amino acid exchanges at the sequence positions selected from T58A or T58Q, L89S, N96D, G117D, L216W and N217D,
e) perhydrolases, whose amino acid sequence matches the amino acid sequences listed in the SEQ ID NO. 8, 10, 12, 14, 16, 18, 20, 22 or 24, increasingly preferably to at least 70%, 72,5%, 75%, 77,5%, 80%, 82,5%, 85%, 87,5%, 90%, 92,5%, 95%, 97,5% and quite particularly preferably to 100%.

Therefore, those perhydrolases are preferably used that are described in the previously unpublished application WO 2004/058961 A1 that is based on the application DE 10260903.9 of the applicant (see above) and whose disclosure is incorporated herein by reference in its entirety. It especially describes perhydrolases, whose amino acid sequence corresponds to that listed in SEQ ID NO. 26, but carries one or a plurality of amino acid exchanges at the sequence positions selected from: 11, 15, 21, 38, 50, 54, 58, 77, 83, 89, 93, 96, 107, 117, 120, 134, 135, 136, 140, 147, 150, 154, 155, 160, 161, 171, 179, 180, 181, 194, 205, 208, 213, 216, 217, 238, 239, 251, 253, 257, 261.

According to the invention, these mutations can be combined with further mutations that for example yield an additional increase in performance or a higher stability towards denaturing agents or higher temperatures.

The coding DNA sequence for the preprotein of subtilisin Carlsberg, an alkaline protease, is listed in SEQ ID NO. 25, and the derived amino acid sequence is listed under SEQ ID NO. 26. This enzyme is naturally formed from B. licheniformis, wherein the first 105 amino acids are split off, i.e. only the last 274 amino acids include the mature protein. As already explained above, this wild type molecule possesses a much too low perhydrolase secondary activity for it to be of interest in the context of the application areas under consideration here. However, by suitable mutations, this enzyme can be provided with a useable perhydrolase activity.

The starting point for obtaining the inventively employable perhydrolase is therefore the protease subtilisin Carlsberg illustrated in SEQ ID NO. 26 of the present application. With the help of these data, this enzyme can be obtained by the use of established molecular biological and biotechnological methods. These generally fall back on the associated nucleotide sequence, which is why this is listed in SEQ ID NO. 25.

The amino acid exchange at the cited positions can be carried out by the use of known molecular biological methods, preferably at the level of the associated nucleotide sequence in the form of point mutations. Suitable commercially available kits for site-specific mutagenesis with the corresponding mismatch primer are, for example the QuickChange® kit from the Stratagene company, La Jolla, USA. Accordingly, genes that already carry a mutation, in particular an inventive mutation, can also be provided with one or a plurality of further inventive mutations, with the result that a large number of inventive variants is accessible.

The inventively employable perhydrolases advantageously exhibit a high specific rate of formation of percarboxylic acid. Advantageously, this is expressed in ppm AO per μg enzyme.

The pH profile of the inventively employable enzymes is advantageously compatible with the pH needed for technical use, as well as with typical products such as detergents and cleaning compositions and hair dyes. In most cases this is an alkaline medium; for this it is advantageous that the starting enzyme for obtaining a preferred inventively employable perhydrolase, the subtilisin Carlsberg, is an alkaline protease. Preferred inventively employable perhydrolases therefore exhibit an optimum pH preferably in the alkaline range of about pH 7 to pH 12, particularly pH 8 to pH 10, depending on the intended technical application.

The temperature optimum of preferred inventively employable perhydrolases likewise depends on the intended technical application and is in the range 20 to 60° C., especially about 30-50° C.

Preferred inventively employable perhydrolases are those whose amino acid sequence corresponds to that listed in SEQ ID NO. 26, but carry one or a plurality of amino acid exchanges at the sequence positions selected from: 11, 58, 77, 89, 96, 117, 120, 134, 135, 136, 140, 147, 150, 161, 208, 216, 217, 238.

Further preferred inventively employable perhydrolases are those whose amino acid sequence corresponds to that listed in SEQ ID NO. 13, but carry one or a plurality of amino acid exchanges at the sequence positions selected from: 58, 89, 96, 117, 216, 217.

Further preferred inventively employable perhydrolases are those exhibiting one or a plurality of amino acid exchanges T58A or T58Q, L89S, N96D, G117D, L216W and N217D.

Here, the following combination of exchanges are preferred:
L89S/L216W/N217D, L216W/N217D, T58A/L89S/L216W/N217D, T58A/G117D/L216W/N217D, T58A/L89S/L216W, T58A/L89S/N96D/L216W, T58A/L216W, T58Q/L89S/L216W and L89S/L216W.

Further preferred inventively employable perhydrolases are those with one of the amino acid sequences listed under SEQ ID NO. 8 (L89S/L216W/N217D), SEQ ID NO. 10 (L216W/N217D), SEQ ID NO. 12 (T58A/L89S/L216W/N217D), SEQ ID NO. 14 (T58A/G117D/L216W/N217D), SEQ ID NO. 16 (T58A/L89S/L216W), SEQ ID NO. 18 (T58A/L89S/N96D/L216W), SEQ ID NO. 20 (T58A/L216W), SEQ ID NO. 22 (T58Q/L89S/L216W) and SEQ ID NO. 24 (L89S/L216W). It should be noted in the sequence listing of the present application, that they concern artificial sequences that have been derived precisely from these point mutations of subtilisin Carlsberg.

In additional, increasingly preferred embodiments of the present invention the inventive enzymatic bleaching systems comprise perhydrolases, whose amino acid sequence matches the amino acid sequences listed in one of the SEQ ID NO. 8, 10,12,14, 16, 18, 20, 22 or 24, increasingly preferably to at least 70%, 72.5%, 75%, 77.5%, 80%, 82.5%, 85%, 87.5%, 90%, 92.5%, 95%, 97.5% and quite particularly preferably to 100%.

In additional preferred embodiments of the present invention the inventive enzymatic bleaching systems comprise perhydrolases that are obtainable by one or a plurality of conservative amino acid exchanges from one of the previously described perhydrolases, preferably within the limits of the homology values for the amino acids or the nucleotide sequences listed herein. Conservative amino acid exchanges are understood to mean exchanges within the following amino acid groups:
aliphatic amino acids: G, A, V, L, I;
sulfur-containing amino acids: C, M;
aromatic amino acids: F, Y, W;
hydroxyl group-containing amino acids: S, T;
amide group-containing amino acids: N, Q;
acidic amino acids: D, E;
basic amino acids: H, K, R, P.

In additional embodiments of the present invention, the inventive enzymatic bleaching systems comprise perhydrolases that are obtainable by derivatization, fragmentation, deletion mutation or insertion mutation of one of the previously described perhydrolases, and precisely within the limits of the above listed homology values for the inventively relevant amino acid sequences.

### DETAILED DESCRIPTION OF THE INVENTION

In the context of the present application, a protein is understood to mean a polymer composed of natural amino acids, which is essentially linear in structure and which assumes in the main a three dimensional structure for carrying out its function. In the present application, the 19 naturally occurring L-amino acids that serve as building blocks of proteins, are designated with the customary international 1 and 3 letter codes.

In the context of the present application, an enzyme is understood to mean a protein that has a specific biocatalytic function.

Numerous proteins are so called preproteins, i.e. formed together with a signal peptide. Included among these is the N-terminal part of the protein, whose function is mainly to guarantee the expulsion of the formed protein from the production cells into the periplasma or the surrounding medium and/or its correct folding. Subsequently, the signal peptide is split off from the remaining protein under natural conditions by a signal peptidase, such that this exercises its original catalytic activity without the first present N-terminal amino acids.

Due to their enzymatic activity, the mature peptides, i.e. the enzymes processed after their production, are preferred over the preproteins for technical applications.

Pro-proteins are inactive preliminary intermediates of proteins. Their precursors with signal sequence are designated as pre-pro-proteins.

In the context of the present application, nucleic acids are understood to mean the molecules that are naturally constructed from nucleotides, which serve as information carriers and code for the linear amino acid sequence in proteins or enzymes. They can be present as a single strand, as a complementary single strand to this single strand or as a double strand. Nucleic acid DNA is preferred as the naturally, long lasting information carrier for molecular biological work. On the other hand, an RNA is formed for the realization of the invention in natural surroundings, such as for example in an expression cell.

In DNA the sequences of both complementary strands have to be taken into account in each of all three possible reading frames. In addition, it has to be taken into account that different codon triplets can code for the same amino acids, with the result that a specific amino acid sequence can be derived from a plurality of different and nucleotide sequences exhibiting possibly only slight identity (degeneracy of the genetic code). Moreover, various organisms exhibit differences in the use of these codons. On these grounds, both amino acid sequences as well as nucleotide sequences have to be included in considerations of the field of protection, and listed nucleotide sequences are only to be regarded as an example of coding for a specific amino acid sequence.

The information unit corresponding to a protein is also designated as a gene in the context of the present application.

The present invention includes the use of recombinant proteins. According to the invention, processes for their manufacture include all gene technical or microbiological processes that are based on the fact that the genes for the proteins of interest are brought in a host organism that is suitable for the production and are transcribed and translated by it. The gene in question is suitably incorporated through vectors, especially expression vectors, but also through those that cause the gene of interest in the host organism to be inserted into an already present genetic element such as the chromosome or another vector. The Functional unit of gene and promoter and possibly additional genetic elements is designated as the expression cassette according to the invention. However, it must not also necessarily be present as a physical unit.

Using today's generally known methods, such as for example chemical synthesis or the polymerase chain reaction (PCR) in combination with molecular biological and/or protein chemical standard methods, it is possible for the person skilled in the art to manufacture, with the help of known DNA sequences and/or amino acid sequences, the corresponding nucleic acids up to complete genes. Such methods are known for example from Sambrook, J., Fritsch, E. F. and Maniatis, T. 2001. Molecular cloning: a laboratory manual, 3rd Edition Cold Spring Laboratory Press.

Modifications of the nucleotide sequence, as can be brought about by known molecular biological methods, are called mutations. Known types depend on the nature of the modification, for example deletion mutations, insertion mutations or substitution mutations or those in which various genes or parts of genes are fused together or recombined; they are gene mutations. The associated organisms are called mutants. The proteins derived from mutated nucleic acids are called variants. Thus, for example deletion-, insertion-, substitution mutations or fusions lead to deletion-, insertion-, substitution mutants or fusion genes and at the protein level to corresponding deletion-, insertion- or substitution variants or fusion proteins.

Fragments are understood to mean all proteins or peptides, which are smaller than natural proteins or those that correspond to completely translated genes, and for example can also be obtained synthetically. Due to their amino acid sequences, they can be assigned to the relevant complete proteins. For example, they can assume the same structure or exercise proteolytic activities or partial activities, such as for example the complexation of a substrate. Fragments and deletion variants of starting proteins are in principle very similar; while fragments depict rather smaller debris, the deletion mutants rather lack only short regions, and therefore only a few partial functions.

At the nucleic acid level, the partial sequences correspond to the fragments.

In the context of the present application, chimeric or hybrid proteins are understood to mean those proteins that are coded from nucleic acid chains that naturally come from different or from the same organism. This procedure is also called recombination mutagenesis. The sense of such a recombination can consist in, for example, providing or modifying a specific enzymatic function with the help of the fused-on protein part. It is irrelevant in the context of the present invention whether such a chimeric protein consists of a single polypeptide chain or a plurality of sub-units, onto which various functions can be distributed.

“Proteins obtained by means of insertion mutation” are understood to mean those variants that have been obtained by known methods of inserting a nucleic acid fragment or protein fragment into the starting sequences. Due to their fundamental similarity, they are classified as chimeric proteins. They differ from those only in the proportion of the size of the unchanged part of the protein to the size of the whole protein. In these insertion mutated proteins, the share of foreign protein is less than in chimeric proteins.

Inversion mutagenesis, meaning a partial reversal of the sequence, can be regarded as a special form of both deletion as well as of insertion. The same is true for new groupings of different molecular parts that differ from the original amino acid sequence. It can be regarded both as a deletion variant, as an insertion variant as well as a shuffling variant of the original protein.

In the context of the present application, derivatives are understood to mean proteins, whose particular amino acid chain has been chemically modified. Such derivatizations can be effected biologically, for example, by the host organism in connection with the protein biosynthesis. Molecular biological methods can be used for this. However, they can also be effected chemically, for example by the chemical transformation of a side chain of an amino acid or by the covalent bonding of another compound onto the protein. This type of compound can also be for example other proteins that are bonded to the inventively employable proteins through a bifunctional chemical compound, for example. These types of modification can influence, for example, the substrate specificity or the binding strength to the substrate or provide a temporary blocking of the enzymatic activity in the case where the attached substance is an inhibitor. This can be meaningful for the storage period, for example. Similarly, derivatization is also understood to mean the covalent bonding to a macromolecular support.

Proteins can also be assimilated to groups of immunologically related proteins by reaction with an antiserum of a specific antibody. The members of a group are characterized in that they possess the same antigen determinant recognized by an antibody.

In the context of the present invention, all enzymes, proteins, fragments and derivatives, in so far as they do not need to be explicitly treated as such, are assimilated under the generic term proteins.

In the context of the present invention, vectors are understood to mean elements that consist of nucleic acids, which comprise a gene of interest as the characterizing nucleic acid region. They are able to establish the gene as a stable genetic element in a species or a cell line over several generations or cell divisions. Vectors, particularly when used in bacteria, especially plasmids, are therefore circular genetic elements. In gene technology, a differentiation is made, on the one hand, between those vectors that serve the storage and thereby to a certain extent also the technical genetic work, the so called cloning vectors, and on the other hand, those that fulfil the function of realizing the gene of interest in the host cells, i.e. to enable the expression of the protein in question. These vectors are called expression vectors.

By comparing with known enzymes, which for example have been deposited in generally accessible data banks, the enzymatic activity of an enzyme under study can be deduced from the amino acid sequence or the nucleotide sequence. This can be qualitatively or quantitatively modified by other regions of the protein, which do not participate in the actual reaction. This can concern, for example, the enzyme stability, the activity, the reaction conditions or the substrate specificity.

This comparison is made by assigning similar sequences in the nucleotide sequences or amino acid sequences of the studied protein with one another. This is called homologization. A tabular assignment of the positions is called the alignment. When analyzing nucleotide sequences, both complementary strands and each of all three possible reading frames have again to be taken into account; the same goes for the degeneracy of the genetic code and the organism-specific codon usage. Alignments have since been drawn up by means of computer programs, such as, for example by the algorithms FASTA or BLAST; this method is described, for example, by D. J. Lipman and W. R. Pearson (1985) in Science, volume 227, pp. 1435-1441. This is preferably carried out with the computer program Vector NTI® Suite 7.0 using the defined default parameters, which is available from InforMax Inc., Bethesda, USA.

A compilation of all matching positions in the compared sequences is called a consensus sequence.

A comparison of this type allows a statement to be made of the similarity or homology of the compared sequences to one another. This is reported in percent identity, i.e. the proportion of identical nucleotides or amino acid residues in the same positions. Another accepted homology term includes the conservative amino acid exchanges in this value. This is then termed the percent similarity. Such statements can refer to the whole protein or gene or only to specific regions.

The construction of an alignment is the first step for defining a sequence space. This hypothetical space includes all sequences obtained by permutation in single positions, which can occur by considering all variations appearing in the relevant single positions of the alignment. Every hypothetically possible protein molecule forms a point in this sequence. For example, two amino acid sequences that each exhibit two different amino acids at only two different positions in a complete identity, therefore establish a sequence space of four different amino acid sequences. A very large sequence space is obtained when additional homologous sequences are each found for single sequences of a space. For those high homologies consisting in pairs, also very low homologous sequences can be recognized as belonging to a sequence space.

Homologous regions of different proteins are defined by matching the amino acid sequence. They can also be characterized by identical functions. This goes as far as complete identities in the smallest region, so called boxes, which include only a few amino acids and mostly exercise essential functions for the overall activity. Functions of the homologous regions are understood to mean the smallest partial functions of the function exercised by the whole protein, such as for example the formation of single hydrogen bonds for complexing a substrate or transition complex.

In the context of the present invention, a recombinant protein is obtained by suitably cloning the nucleic acids into a vector. The molecular biological dimension of the invention accordingly consists in vectors with the genes for the corresponding proteins. For example, they can include those that derive from bacterial plasmids, from viruses or from bacteriophages, or essentially synthetic vectors or plasmids with elements from the most different origin. Vectors with each of the additional available genetic elements are able to establish themselves in the relevant host cells for several generations to as far as stable units. Accordingly, in the context of the invention, it is irrelevant whether they establish themselves extrachromosomally as their own units or are integrated into a chromosome. Whichever of the numerous systems known from the prior art is selected, depends on the individual case. The achievable number of copies, the available selection systems, principally among them resistance to antibiotics, or the ability to cultivate host cells that can take up the vectors, for example, can be decisive.

The vectors form suitable starting points for molecular biological and biochemical investigations of the relevant genes or associated proteins and for inventive further developments and finally for the amplification and production of inventively employable proteins. In this respect, they illustrate embodiments of the present invention, as the sequences of the resulting inventively employable nucleic acid regions each lie within the homology regions more precisely designated above.

Cloning vectors are specific form of vectors. Besides storage, biological amplification or selection of genes of interest for the characterization of the relevant genes, they are suitable, for example, for building a restriction map or sequencing. Cloning vectors are a transportable and storable form of DNA that can be used to obtain a protein. They are also preferred starting points for molecular biological techniques that are not linked to cells, such as for example the polymerase chain reaction.

Expression vectors are chemically similar to cloning vectors, but differ in each partial sequence that enables them to replicate host organisms optimized for the production of proteins and to bring the resulting gene to expression there. Expression vectors that themselves carry the genetic elements required for expression are particularly suitable. The expression is influenced, for example by promoters that regulate the transcription of the genes. Thus, the expression can occur by means of the natural, original, localized promoter with this gene, but also after gene technical fusion, both by means of a prepared promoter of the host cell on the expression vector and also by a modified or a completely other promoter of another organism.

Those expression vectors that are particularly suitable for obtaining proteins are regulatable by changing the conditions of culture or by adding certain compounds, such as for example the cell density or specific factors. Expression vectors permit the associated protein to be produced heterologously, i.e. in a different organism as that from which it can be naturally obtained. A homologous protein production from a host organism that naturally expresses the gene over an appropriate vector lies within the field of protection of the present invention. This can have the advantage that natural, modification reactions in a context of the translation on the resulting protein can be carried out in the same way as they would normally be.

In the context of the present invention, cell-free expression systems can also be important, in which the protein biosynthesis is reconstructed in vitro. Such expression systems are also established in the prior art.

The in vivo synthesis of an inventively employable enzyme, i.e. by living cells, requires the transfer of the associated gene into a host cell, its so called transformation. In principle, all organisms, i.e. prokaryotes or eukaryotes, are suitable host cells. Those host cells are preferred, which can be genetically handled with ease, for example in relation to the transformation with the expression factor and its stable establishment, for example single cell fungi or bacteria. In addition, preferred host cells are those with a good microbiological and biotechnological handleability. For example, this relates to ease of cultivation, high growth rates, low demands on fermentation media and good production rates and secretion rates for foreign proteins. Frequently, the optimum expression system for the individual case must be experimentally determined from the abundance of different systems available from the prior art. Each of the inventively employable proteins can be obtained in this way from a great number of host organisms.

In the context of the invention, such host cells are also important, that can be regulated in their activity due to the genetic regulation elements that are, for example, made available to the expression vector, but which can also be already present in these cells. For example, they can be stimulated to expression by the controlled addition of chemical compounds that serve as activators, by changing the cultivation conditions or by attaining a specific cell density. This enables a very economical production of the products of interest.

Preferred host cells are prokaryotic or bacterial cells. Bacteria, in comparison with eukaryotes, generally have shorter generation times and lesser demands on the cultivation conditions. This enables cost effective processes for obtaining inventively employable proteins to be established. In gram-negative bacteria, such as Escherichia Coli (E. coli), a large number of proteins are secreted into the periplasmatic space, i.e. into the compartment between both the membranes that encapsulate the cells. This can be advantageous for specific applications. On the other hand, gram-positive bacteria, such as bacilli or actinomycetes or other representatives of the actinomycetes, possess no external membrane, such that secreted proteins are immediately emitted into the alimentation medium surrounding the cells, from which according to another preferred embodiment the expressed inventively employable proteins can be directly purified.

Expression systems illustrate a variant of this experimental principle, in which additional genes, for example those that are made available on other vectors, influence the production of inventively employable proteins. They can be modified gene products or those intended to be purified together with the inventively employable protein, for example to influence its enzymatic function. They can be other proteins or enzymes, for example, inhibitors or such elements that influence the interactions with various substrates.

Due to the far-reaching experience obtained with regard to, for example, the molecular biological methods and the cultivation with coliform bacteria, they are preferred for the expression of the inventively employable proteins. Those of the genera Escherichia Coli, especially non-pathogenic strains suitable for the biotechnological production, are particularly preferred.

Representative members of these genera are the K12 derivatives and the B-strains of Escherichia Coli. Strains that can be derived from them according to known genetic and/or microbiological methods and thereby can be considered as their derivatives, possess the most important significance for genetic and microbiological work and are preferably employed for the development of inventive processes. Such derivatives can be modified for example through deletion mutagenesis or insertion mutagenesis in regard to their demands on the conditions of culture, exhibit other or additional selection markers or express other or additional proteins. In particular, they can be such derivatives that express additional economically interesting proteins in addition to the inventively employable manufactured proteins.

Preferred microorganisms are also those, which have been obtained by transformation with one of the vectors described above. This can concern cloning vectors, for example, which have been inserted into any bacterial strain for storage and/or modification. In general, such steps are widespread in the storage and further development of the genetic elements under consideration. As the concerned genetic elements from these microorganisms can be directly transferred into gram-negative bacteria for expression, the previous transformation products can also fulfil the subject matter of the invention under consideration.

Eukaryotic cells can also be suitable for the production of inventively employable proteins. Examples of these are fungi like actinomycetes or yeasts like saccharomyces or kluyveromyces. For example, this can be particularly advantageous if the proteins should be subjected to specific modifications in connection with their synthesis, which permit such systems. For example, these include the binding of low molecular weight compounds such as docking membranes or oligosaccharides.

The host cells are cultivated and fermented in a conventional manner, for example in discontinuous or continuous systems. In the first case, a suitable nutrient medium is inoculated with the microorganisms and the product is harvested from the medium after an experimentally determined time. Continuous fermentations are characterized by the attainment of a flow equilibrium, in which, for a comparatively long time, cells partially die off but also grow again, and product can be removed from the medium.

Fermentation processes per se are well known from the prior art and represent the actual industrial production step; followed by a suitable purification method.

All fermentation processes that are based on one of the above-explained processes for manufacturing recombinant proteins can be used in the scope of the present invention.

Here the optimal conditions for the production process, the host cells and/or the protein being produced have to be experimentally determined by the person skilled in the art with the help of the previously optimised culture conditions of the strains in question, for example, in regard to fermentation volumes, medium composition, oxygen demand or stirring rate.

Fermentation processes, wherein the fermentation is carried out with a supply strategy, can also be considered. For this the ingredients of the medium that are used up by the ongoing cultivation are fed in; this is also known as a feed strategy. Considerable increases in both the cell density and in the dry biomass and/or above all in the activity of the protein of interest can be achieved by this.

In analogy with this, the fermentation can also be designed in such a way that unwanted metabolic products can be filtered off or be neutralized by the addition of buffer or matching counter ions.

The manufactured protein can be subsequently harvested from the fermentation medium. This fermentation process is preferred over the product purification from the dry mass, but requires the availability of suitable secretion markers and transport systems.

Without secretion, the purification of the proteins from the cell mass is possibly required and various processes are known for this, such as precipitation with e.g. ammonium sulfate or ethanol, or chromatographic purification, when required to homogeneity. However, the majority of the described techniques should be done with an enriched, stabilized preparation.

All of the above listed elements can be combined in processes to manufacture the inventively employable proteins. A great many possible combinations of process steps are conceivable for each inventively employable protein. The optimum process has to be determined experimentally for each particular case.

The inventively employable proteins can be produced in sufficient quantities for industrial use by expression or cloning.

The inventively preferred employable choline oxidases exhibit a pH optimum preferably in the almost neutral to weakly alkaline range of about pH 6 to pH 10, particularly preferably pH 7 to pH 9. The activity of such enzymes is usually expressed in U, the unit corresponding to the quantity of enzyme that generates 1 μmol of hydrogen peroxide (H₂O₂) at a defined pH and a defined temperature in 1 minute. For the inventively employable choline oxidases, this refers to a pH of 9.5 and a temperature of 30° C. in the process described in example 6.

The temperature optimum of the inventively employable choline oxidases is in the range about 20 to 60° C., particularly at about 30° C.

Preferably, the amount of inventively employable choline oxidase that is employed is such that the total composition exhibits an oxidase activity of 3 U/g to 20 000 U/g, preferably 5 U/g to 20 000 U/g, particularly 10 U/g to 15 000 U/g, particularly preferably 10 U/g to 1000 U/g and quite particularly preferably 20 to 60 U/g. The unit (U) is defined as the quantity of oxidase that forms 1 μmol hydrogen peroxide in one minute.

Compositions with oxidase activities in the cited ranges possess a sufficiently rapid hydrogen peroxide release for usual European machine-washing processes, whereas an increase in the comprised amount of oxidase to higher activities does not generally produce any corresponding increase in bleach performance.

The quantity of the substrate for the oxidase comprised in the inventive detergent depends on the quantity of hydrogen peroxide required to obtain the desired bleach result. As an indication, each mole of reacted substrate releases up to two moles of hydrogen peroxide in enzyme substrate systems. The presence of about 0.05 wt. % to 1 wt. % of the substrate in the detergent-, bleach- or cleaning liquor is generally sufficient to obtain a good bleaching result.

The inventive enzymatic bleaching system can be advantageously incorporated into appropriate agents.

Accordingly, a separate subject matter of the invention is constituted by body care compositions, hair shampoos, mouth-, tooth- or denture care compositions, care compositions for tooth braces, cosmetics, therapeutics, textile detergents, cleaning compositions, rinse agents, textile detergents for washing machines, detergents for hand washing, dish washing detergents, automatic dishwasher detergents, disinfectants and compositions for bleaching or disinfecting filter media, textiles, furs/pelts, paper, hides or leather, which comprise one of the above described inventive bleaching systems.

Among these, preferred compositions are textile detergents, bleaching agents or cleaning compositions, preferably, detergents for machine-washing textiles or automatic dishwasher detergents.

Firstly, this is because they concern economically particularly important application areas for this type of enzymatic bleaching system. Secondly, it could be demonstrated in example 1 of the present application that such systems used in textile cleaning demonstrate advantages over systems from the prior art.

These types of inventive composition advantageously comprise additional bleach-, detergent- or cleaning ingredients, such as, for example surfactants or builders. These are described in more detail below.

A further preferred inventive composition exhibits an oxidase activity of 1 to 20 000 U/g, preferably 10 to 10 000 U/g, particularly preferably 100 to 1000 U/g and a perhydrolase concentration of 0.5 to 100 μg/ml, preferably 1 to 75 μg/ml, particularly preferably 10 to 50 μg/ml.

The definitions for these activity values have already been given above.

A further preferred inventive composition is present as a free-flowing powder with a bulk density of 300 to 1200 g/l, preferably 400 to 1000 g/l, particularly preferably 500 to 900 g/l.

Bulk densities of this type have become established in the prior art especially for textile detergents used in washing machines, and the consumer as well as the manufacturers of the washing machines accommodate this.

Not less preferred is an inventive composition in the form of a pasty or liquid detergent.

They can be non-aqueous liquid detergents, aqueous detergents on non-aqueous or water-containing pastes. These presentation forms enjoy increased customer favor due to their ease of dosing and their oftentimes lower propensity to forming residues.

The inventive detergent or bleaching agent can be packaged in an airtight container, from which it is released shortly before use or during the washing process. In particular, the oxidase and perhydrolase and/or the corresponding substrates can be encapsulated with a substance that is impermeable to the enzyme and/or its substrate at room temperature or in the absence of water, and which becomes permeable to the enzyme and/or its substrate under the conditions of use of the composition.

A further preferred inventive composition comprises, in addition to the bleaching system,
5 wt. % to 70 wt. %, particularly 10 wt. % to 50 wt. % surfactant,
10 wt. % to 65 wt. %, particularly 12 wt. % to 60 wt. % of water-soluble, water-dispersible inorganic builder,
1 wt. % to 10 wt. %, particularly 2 wt. % to 8 wt. % of water-soluble organic builders,
not more than 15 wt. % solid inorganic and/or organic acids or their salts,
not more than 5 wt. % heavy metal sequestrants,
not more than 5 wt. % graying inhibitors,
not more than 5 wt. % color transfer inhibitors and
not more than 5 wt. % foam inhibitor.

These are ingredients that have proven to be additionally active agents along with an inventive enzymatic bleaching system, in particular for detergents and cleaning compositions.

A further preferred inventive composition additionally comprises further enzymes, in particular proteases, amylases, cellulases, hemicellulases, further oxidoreductases and/or lipases.

These, due to their specific hydrolytic properties contribute especially to the cleaning power of the formulations, cellulase being particularly valued for its action on textile surfaces. This will be discussed in more detail below.

Due to their high technical importance, the different aspects and other ingredients of the inventive, that is the detergent and cleaning composition characterized by the above described bleaching system, will now be described in order to amplify in detail the above described particularly preferred embodiments.

There will be no overall distinction made between textiles and hard surfaces as the material to be washed. The available choices, in particular for the conditions required for the various ingredients, such as, for example temperature, pH, ion strength, redox conditions or mechanical influences, should be optimised for each cleaning problem. Thus, usual temperatures for detergents and cleaning compositions are in the range 10° C. for manual compositions over 40° C. and 60° C. up to 95° C. for machine compositions or for industrial applications. As the temperature is mostly steplessly adjustable in modern washing machines and dishwashers, all intermediate steps of temperature are included. Preferably, the ingredients of the composition are harmonized with each other. Synergies in regard to the cleaning power are preferred.

An inventive bleaching system can be used both in compositions for large-scale end users or industrial users and also in products for the private consumer, wherein all types of cleaning compositions established in the prior art also represent embodiments of the present invention. This includes for example concentrates and compositions to be used without dilution—for use on a commercial scale in washing machines or in hand washing or hand cleaning. These include, for example, detergents for fabrics, carpets or natural fibers, for which the term “detergent” is used in the present invention. These also include, for example, dishwashing detergents for dishwashing machines or manual dishwashing detergents or cleansers for hard surfaces, such as metal, glass, china, ceramic, tiles, stone, painted surfaces, plastics, wood or leather, for which the term “cleaning composition” is used in the present invention.

Embodiments of the present invention include all established and/or all appropriate presentation forms. These include for example solid, powdered, liquid, gel or pasty agents, optionally from a plurality of phases, compressed or non-compressed; further included are for example: extrudates, granulates, tablets or pouches, both in bulk and also packed in portions.

In inventive compositions, the inventive bleaching system is combined with individual or a plurality of the following ingredients: non-ionic, anionic and/or cationic surfactants, (optionally additional) bleaching agents, bleach activators, bleach catalysts, builders and/or cobuilders, solvents, thickeners, sequestrants, electrolytes, optical brighteners, graying inhibitors, corrosion inhibitors, especially silver protectants, soil release agents, color transfer inhibitors, foam inhibitors, abrasives, colorants, fragrances, antimicrobials, UV stabilizers, enzymes such as for example proteases, amylases, lipases, cellulases, hemicellulases or oxidases, stabilizers, especially enzyme stabilizers, and other components, which are known from the prior art.

Preferred non-ionic surfactants are alkoxylated, advantageously ethoxylated, particularly primary alcohols preferably containing 8 to 18 carbon atoms and, on average, 1 to 12 moles of ethylene oxide (EO) per mole of alcohol, in which the alcohol group may be linear or, preferably, methyl-branched in the 2-position or may contain linear and methyl-branched groups in the form of the mixtures typically present in oxoalcohol groups. Particularly preferred are, however, alcohol ethoxylates with linear alcohol groups of natural origin with 12 to 18 carbon atoms, e.g. from coco-, palm-, tallow- or oleyl alcohol, and an average of 2 to 8 EO per mole alcohol. Exemplary preferred ethoxylated alcohols include C₁₂₋₁₄-alcohols with 3 EO or 4EO, C₉₋₁₁-alcohols with 7 EO, C₁₃₋₁₅-alcohols with 3 EO, 5 EO, 7 EO or 8 EO, C₁₂₋₁₈-alcohols with 3 EO, 5 EO or 7 EO and mixtures thereof, as well as mixtures of C₁₂₋₁₄-alcohol with 3 EO and C12-18-alcohol with 5 EO. The cited degrees of ethoxylation constitute statistically average values that can be a whole or a fractional number for a specific product. Preferred alcohol ethoxylates have a narrowed homolog distribution (narrow range ethoxylates, NRE). In addition to these non-ionic surfactants, fatty alcohols with more than 12 EO can also be used. Examples of these are tallow fatty alcohol with 14 EO, 25 EO, 30 EO or 40 EO.

Another class of preferred non-ionic surfactants which may be used, either as the sole non-ionic surfactant or in combination with other non-ionic surfactants are alkoxylated, preferably ethoxylated or ethoxylated and propoxylated fatty acid alkyl esters preferably containing 1 to 4 carbon atoms in the alkyl chain, in particular fatty acid methyl esters.

A further class of non-ionic surfactants, which can be advantageously used, are the alkyl polyglycosides (APG). Suitable alkyl polyglycosides satisfy the general formula RO(G), where R is a linear or branched, particularly 2-methyl-branched, saturated or unsaturated aliphatic group containing 8 to 22 and preferably 12 to 18 carbon atoms and G stands for a glycose unit containing 5 or 6 carbon atoms, preferably glucose. Here, the degree of glycosidation z is between 1.0 and 4.0, preferably between 1.0 and 2.0 and particularly between 1.1 and 1.4. Linear alkyl polyglucosides are preferably employed, that is alkyl polyglycosides, in which the polyglycosyl group is a glucose group and the alkyl group is an n-alkyl group.

Non-ionic surfactants of the amine oxide type, for example N-coco alkyl-N,N-dimethylamine oxide and N-tallow alkyl-N,N-dihydroxyethylamine oxide, and the fatty acid alkanolamides may also be suitable. The quantity of these non-ionic surfactants is preferably no more than the quantity in which the ethoxylated fatty alcohols are used and, particularly no more than half that quantity.

Other suitable surfactants are polyhydroxyfatty acid amides corresponding to the Formula (II), 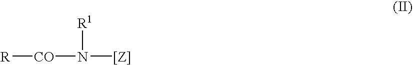
in which RCO stands for an aliphatic acyl group with 6 to 22 carbon atoms, R¹ for hydrogen, an alkyl or hydroxyalkyl group with 1 to 4 carbon atoms and [Z] for a linear or branched polyhydroxyalkyl group with 3 to 10 carbon atoms and 3 to 10 hydroxyl groups. The polyhydroxyfatty acid amides are known substances, which may normally be obtained by reductive amination of a reducing sugar with ammonia, an alkylamine or an alkanolamine and subsequent acylation with a fatty acid, a fatty acid alkyl ester or a fatty acid chloride.

The group of polyhydroxyfatty acid amides also includes compounds corresponding to the Formula (III), 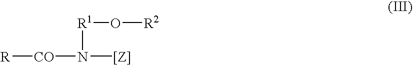
in which R is a linear or branched alkyl or alkenyl group containing 7 to 12 carbon atoms, R¹ is a linear, branched or cyclic alkyl group or an aryl group containing 2 to 8 carbon atoms and R² is a linear, branched or cyclic alkyl group or an aryl group or an oxyalkyl group containing 1 to 8 carbon atoms, C₁₋₄ alkyl or phenyl groups being preferred, and [Z] is a linear polyhydroxyalkyl group, of which the alkyl chain is substituted by at least two hydroxy groups, or alkoxylated, preferably ethoxylated or propoxylated derivatives of that group.

[Z] is preferably obtained by reductive amination of a sugar, for example glucose, fructose, maltose, lactose, galactose, mannose or xylose. The N-alkoxy- or N-aryloxy-substituted compounds may then be converted into the required polyhydroxyfatty acid amides by reaction with fatty acid methyl esters in the presence of an alkoxide as catalyst.

Exemplary suitable anionic surfactants are those of the sulfonate and sulfate type. Suitable surfactants of the sulfonate type are, advantageously C₉₋₁₃-alkylbenzene sulfonates, olefin sulfonates, i.e. mixtures of alkene- and hydroxyalkane sulfonates, and disulfonates, as are obtained, for example, from C₁₂₋₁₈-monoolefins having a terminal or internal double bond, by sulfonation with gaseous sulfur trioxide and subsequent alkaline or acidic hydrolysis of the sulfonation products. Those alkane sulfonates, obtained from C₁₂₋₁₈ alkanes by sulfochlorination or sulfoxidation, for example, with subsequent hydrolysis or neutralization, are also suitable. The esters of α-sulfofatty acids (ester sulfonates), e.g. the α-sulfonated methyl esters of hydrogenated coco-, palm nut- or tallow acids are likewise suitable.

Further suitable anionic surfactants are sulfated fatty acid esters of glycerine. They include the mono-, di- and triesters and also mixtures of them, such as those obtained by the esterification of a monoglycerine with 1 to 3 moles fatty acid or the transesterification of triglycerides with 0.3 to 2 moles glycerine. Preferred sulfated fatty acid esters of glycerol in this case are the sulfated products of saturated fatty acids with 6 to 22 carbon atoms, for example caproic acid, caprylic acid, capric acid, myristic acid, lauric acid, palmitic acid, stearic acid or behenic acid.

Preferred alk(en)yl sulfates are the alkali and especially sodium salts of the sulfuric acid half-esters derived from the C₁₂-C₁₈ fatty alcohols, for example from coconut butter alcohol, tallow alcohol, lauryl, myristyl, cetyl or stearyl alcohol or from C₁₀-C₂₀ oxo alcohols and those half-esters of secondary alcohols of these chain lengths. Additionally preferred are alk(en)yl sulfates of the said chain lengths, which contain a synthetic, straight-chained alkyl group produced on a petro-chemical basis and which show similar degradation behaviour to the suitable compounds based on fat chemical raw materials. The C₁₂-C₁₆ alkyl sulfates and C₁₂-C₁₅ alkyl sulfates and C₁₄-C₁₅ alkyl sulfates are preferred on the grounds of laundry performance. 2,3-Alkyl sulfates are also suitable anionic surfactants.

Sulfuric acid mono-esters derived from straight-chained or branched C₇₋₂₁ alcohols ethoxylated with 1 to 6 moles ethylene oxide are also suitable, for example 2-methyl-branched C₉₋₁₁ alcohols with an average of 3.5 mole ethylene oxide (EO) or C₁₂₋₁₈ fatty alcohols with 1 to 4 EO. Due to their high foaming performance, they are only used in fairly small quantities in cleaning compositions, for example in amounts of up to 5% by weight, usually from 1 to 5% by weight.

Other suitable anionic surfactants are the salts of alkylsulfosuccinic acid, which are also referred to as sulfosuccinates or esters of sulfosuccinic acid and the monoesters and/or di-esters of sulfosuccinic acid with alcohols, preferably fatty alcohols and especially ethoxylated fatty alcohols. Preferred sulfosuccinates contain C₈₋₁₈ fatty alcohol groups or mixtures of them. Especially preferred sulfosuccinates comprise a fatty alcohol group derived from ethoxylated fatty alcohols and may be considered as non-ionic surfactants (see description below). Once again the especially preferred sulfosuccinates are those, whose fatty alcohol groups are derived from ethoxylated fatty alcohols with narrow range distribution. It is also possible to use alk(en)ylsuccinic acid with preferably 8 to 18 carbon atoms in the alk(en)yl chain, or salts thereof.

Soaps in particular can be considered as further anionic surfactants. Saturated fatty acid soaps are suitable, such as the salts of lauric acid, myristic acid, palmitic acid, stearic acid, hydrogenated erucic acid and behenic acid, and especially soap mixtures derived from natural fatty acids such as coconut oil fatty acid, palm kernel oil fatty acid or tallow fatty acid.

Anionic surfactants, including soaps may be in the form of their sodium, potassium or ammonium salts or as soluble salts of organic bases, such as mono-, di- or triethanolamine. Preferably, the anionic surfactants are in the form of their sodium or potassium salts, especially in the form of sodium salts.

The surfactants can be comprised in the inventive cleaning compositions or detergents in an amount of preferably 5 to 50 wt. %, particularly 8 to 30 wt. %, based on the finished composition.

Inventive compositions can comprise additional bleaching agent. Among the compounds, which serve as bleaches and liberate H₂O₂ in water, sodium percarbonate, sodium perborate tetrahydrate and sodium perborate monohydrate are of particular importance. Examples of further bleaching agents that may be used are peroxypyrophosphates, citrate perhydrates and H₂O₂-liberating peracidic salts or peracids, such as persulfates or persulfuric acid. The urea peroxyhydrate percarbamide that can be described by the formula H₂N—CO—NH₂.H₂O₂ is also suitable. Particularly when agents are used to clean hard surfaces, for example for automatic dishwashers, they can, if desired, also comprise bleaching agents from the group of organic bleaching agents, although in principal they can also be used for washing textiles. Typical organic bleaching agents are the diacyl peroxides, such as e.g. dibenzoyl peroxide. Further typical organic bleaching agents are the peroxy acids, wherein the alkylperoxy acids and the arylperoxy acids may be named as examples. Preferred representatives that can be added are peroxybenzoic acid and ring-substituted derivatives thereof, such as alkyl peroxybenzoic acids, but also peroxy-α-naphthoic acid and magnesium monoperphthalate, the aliphatic or substituted aliphatic peroxy acids, such as peroxylauric acid, peroxystearic acid, ε-phthalimidoperoxycaproic acid [phthaloiminoperoxyhexanoic acid PAP)], o-carboxybenzamidoperoxycaproic acid, N-nonenylamido peradipic acid and N-nonenylamido persuccinates and aliphatic and araliphatic peroxydicarboxylic acids, such as 1,12-diperoxycarboxylic acid, 1,9-diperoxyazelaic acid, diperoxysebacic acid, diperoxybrassylic acid, diperoxyphthalic acids, 2-decyldiperoxybutane-1,4-dioic acid, N,N-terephthaloyl-di(6-aminopercaproic acid).

The bleach agent content of the composition is preferably 1 to 40 wt. % and particularly 10 to 20 wt. %, perborate monohydrate or percarbonate being advantageously used. A synergistic use of amylase with percarbonate or of amylase with percarboxylic acid is disclosed in the applications WO 99/63036 and WO 99/63037.

The preparations can also comprise bleach activators in order to achieve an improved bleaching action for washing temperatures of 60° C. and below and particularly during the pre-treatment wash. Bleach activators, which can be used are compounds which, under perhydrolysis conditions, yield aliphatic peroxycarboxylic acids having preferably 1 to 10 carbon atoms, in particular 2 to 4 carbon atoms, and/or optionally substituted perbenzoic acid. Substances, which carry O-acyl and/or N-acyl groups of said number of carbon atoms and/or optionally substituted benzoyl groups, are suitable. Preference is given to polyacylated alkylenediamines, in particular tetraacetyl ethylene diamine (TAED), acylated triazine derivatives, in particular 1,5-diacetyl-2,4-dioxohexahydro-1,3,5-triazine (DADHT), acylated glycolurils, in particular tetraacetyl glycoluril (TAGU), N-acylimides, in particular N-nonanoyl succinimide (NOSI), acylated phenol sulfonates, in particular n-nonanoyl- or isononanoyloxybenzene sulfonate (n- or iso-NOBS), carboxylic acid anhydrides, in particular phthalic anhydride, acylated polyhydric alcohols, in particular triacetin, ethylene glycol diacetate and 2,5-diacetoxy-2,5-dihydrofuran and the enol esters known from the German Patent applications DE-A-196 16 693 and DE-A-196 16 767 and acetylated sorbitol and mannitol or their mixtures (SORMAN) described in the European Patent application EP-A-0 525 239, acylated sugar derivatives, in particular pentaacetylglucose (PAG), pentaacetylfructose, tetraacetylxylose and octaacetyllactose as well as acetylated, optionally N-alkylated glucamine and gluconolactone, triazole or triazole derivatives and/or particulate caprolactams and/or caprolactam derivatives, preferably N-acylated lactams, for example N-benzoylcaprolactam and N-acetylcaprolactam, which are known from the international Patent applications WO-A-94/27970, WO-A-94/28102, WO-A-94/28103, WO-A-95/00626, WO-A-95/14759 and WO-A-95/17498. The hydrophilically substituted acylacetals, known from the German Patent application DE 196 16 769 and the acyllactams described in the German Patent application DE 196 16 770 as well as the international Patent application WO-A-95/14075 are also preferably used. The combinations of conventional bleach activators known from the German Patent application DE 44 43 177 can also be used. Nitrile derivatives such as cyanopyridines, nitrilequats, for example N-alkyl ammonium acetonitrile, and/or cyanamide derivatives can also be used. Preferred bleach activators are sodium 4-(octanoyloxy)benzene sulfonate, n-nonanoyl- or isononanoyloxybenzene sulfonate (n- or iso-NOBS), undecenoyloxybenzene sulfonate (UDOBS), sodium dodecanoyloxybenzene sulfonate (DOBS), decanoyloxybenzoic acid (DOBA, OBC 10) and/or dodecanoyloxybenzene sulfonate (OBS 12), and N-methylmorpholinum acetonitrile (MMA). These types of bleach activators are comprised in the usual quantity range of 0.01 to 20 wt. %, preferably in amounts of 0.1 wt. % to 15 wt. %, particularly 1 wt. % to 10 wt. %, based on the total composition.

In addition to, or instead of the conventional bleach activators mentioned above, so-called bleach catalysts may also be incorporated. These substances are bleach-boosting transition metal salts or transition metal complexes such as, for example, manganese-, iron-, cobalt-, ruthenium- or molybdenum-salen or -carbonyl complexes. Manganese, iron, cobalt, ruthenium, molybdenum, titanium, vanadium and copper complexes with nitrogen-containing tripod ligands, as well as cobalt-, iron-, copper- and ruthenium-amine complexes may also be employed as the bleach catalysts, wherein those compounds that are described in DE 197 09 284 A1 are preferably employed. According to WO 99/63038, acetonitrile derivatives, and according to WO 99/63041, bleach activating transition metal complexes in combination with amylases, are also capable of developing a bleach activating effect.

Generally, inventive compositions comprise one or more builders, in particular zeolites, silicates, carbonates, organic cobuilders and—where there are no ecological grounds against their use—also phosphates. The last are particularly preferred builders employed in cleaning compositions for automatic dishwashers.

Suitable silicate builders are the crystalline, layered sodium silicates corresponding to the general formula NaMSiₓO₂ₓ₊₁ yH₂O, wherein M is sodium or hydrogen, x is a number from 1.6 to 4, preferably 1.9 to 4.0 and y is a number from 0 to 20, preferred values for x being 2, 3 or 4. These types of crystalline layered silicates are described, for example, in the European Patent application EP 0 164 514. Preferred crystalline layered silicates of the given formula are those in which M stands for sodium and x assumes the values 2 or 3. Both β- and also δ-sodium disilicates Na₂Si₂O₅•yH₂O are particularly preferred. These types of compounds are commercially available, for example, under the designation SKS® (Clariant). SKS-6® is mainly a δ-sodium disilicate with the formula Na₂Si₂O₅•yH₂O, and SKS-7® is mainly the β-sodium disilicate. On reaction with acids (e.g. citric acid or carbonic acid), δ-sodium silicate affords Kanemit NaHSi₂O₅•yH2O, commercially available under the trade names SKS-9® and SKS-10® (Clariant). It can also be advantageous to chemically modify these layered silicates. The alkalinity, for example, of the layered silicates can be suitably modified. In comparison with the δ-sodium disilicate, layered silicates, doped with phosphate or carbonate, exhibit a different crystal morphology, dissolve more rapidly and show an increased calcium binding ability. Examples are layered silicates of the general formula x Na₂O•y SiO₂•z P₂O₅ in which the ratio x to y corresponds to a number 0.35 to 0.6, the ratio x to z a number from 1.75 to 1200 and the ratio y to z a number from 4 to 2800, described in the patent application DE 196 01 063. The solubility of the layered silicates can also be increased by employing particularly finely dispersed layered silicates. Compounds of the crystalline layered silicates with other ingredients can also be used. Compounds with cellulose derivatives, which possess advantages in the disintegration action, and which are particularly used in detergent tablets, as well as compounds with polycarboxylates, for example citric acid or polymeric polycarboxylates, for example copolymers of acrylic acid can be particularly cited in this context.

Other useful builders are amorphous sodium silicates with a modulus (Na₂O:SiO₂ ratio) of 1:2 to 1:3.3, preferably 1:2 to 1:2.8 and especially 1:2 to 1:2.6, which dissolve with a delay and exhibit multiple wash cycle properties. The delay in dissolution compared with conventional amorphous sodium silicates can have been obtained in various ways, for example by surface treatment, compounding, compressing/compacting or by over-drying. In the context of this invention, the term “amorphous” also means “X-ray amorphous”. In other words, the silicates do not produce any of the sharp X-ray reflexes typical of crystalline substances in X-ray diffraction experiments, but at best one or more maxima of the scattered X-radiation, which have a width of several degrees of the diffraction angle. However, particularly good builder properties may even be achieved where the silicate particles produce indistinct or even sharp diffraction maxima in electron diffraction experiments. This is to be interpreted to mean that the products have microcrystalline regions between 10 and a few hundred nm in size, values of up to at most 50 nm and especially up to at most 20 nm being preferred. Compacted/densified amorphous silicates, compounded amorphous silicates and over dried X-ray-amorphous silicates are particularly preferred.

Of the suitable fine crystalline, synthetic zeolites containing bound water, zeolite A and/or P are preferred. Zeolite MAP® (commercial product of the Crosfield company), is particularly preferred as the zeolite P. However, zeolite X and mixtures of A, X, Y and/or P are also suitable. Commercially available and preferably used in the context of the present invention is, for example, also a co-crystallizate of zeolite X and zeolite A (ca. 80 wt. % zeolite X), which is marketed by CONDEA Augusta S.p.A. under the trade name VEGOBOND AX® and which can be described by the Formula
nNa₂O•(1-n)K₂O•Al₂O₃•(2-2.5)SiO₂•(3.5-5.5)H₂O

Suitable zeolites have a mean particle size of less than 10 μm μvolume distribution, as measured by the Coulter Counter Method) and comprise preferably 18 to 22% by weight and more preferably 20 to 22% by weight of bound water.

Naturally, the generally known phosphates can also be added as builders, in so far that their use should not be avoided on ecological grounds. In the detergent and cleaning agent industry, among the many commercially available phosphates, the alkali metal phosphates are the most important and pentasodium or pentapotassium triphosphates (sodium or potassium tripolyphosphate) are particularly preferred.

“Alkali metal phosphates” is the collective term for the alkali metal (more particularly sodium and potassium) salts of the various phosphoric acids, in which metaphosphoric acids (HPO₃)ₙ and orthophosphoric acid (H₃PO₄) and representatives of higher molecular weight can be differentiated. The phosphates combine several inherent advantages: they act as alkalinity sources, prevent lime deposits on machine parts and lime incrustations in fabrics and, in addition, contribute towards the cleaning effect.

Sodium dihydrogen phosphate NaH2PO4 exists as the dihydrate (density 1.91 gcm⁻³, melting point 60° C.) and as the monohydrate (density 2.04 gcm⁻³). Both salts are white, readily water-soluble powders that on heating, lose the water of crystallization and at 200° C. are converted into the weakly acidic diphosphate (disodium hydrogen diphosphate, Na₂H₂P₂O₇) and, at higher temperatures into sodium trimetaphosphate (Na₃P₃O₉) and Maddrell's salt (see below). NaH₂PO₄ shows an acidic reaction. It is formed by adjusting phosphoric acid with sodium hydroxide to a pH value of 4.5 and spraying the resulting “mash”. Potassium dihydrogen phosphate (primary or monobasic potassium phosphate, potassium biphosphate, KDP), KH₂PO₄, is a white salt with a density of 2.33 gcm⁻³, has a melting point of 253° C. [decomposition with formation of potassium polyphosphate (KPO₃)ₓ] and is readily soluble in water.

Disodium hydrogen phosphate (secondary sodium phosphate), Na₂HPO₄, is a colorless, very readily water-soluble crystalline salt. It exists in anhydrous form and with 2 mol (density 2.066 gcm⁻³, water loss at 95° C.), 7 mol (density 1.68 gcm⁻³, melting point 48° C. with loss of 5 H₂O) and 12 mol of water (density 1.52 gcm⁻³, melting point 35° with loss of 5 H₂O), becomes anhydrous at 100° C. and, on fairly intensive heating, is converted into the diphosphate Na₄P₂O₇. Disodium hydrogen phosphate is prepared by neutralization of phosphoric acid with soda solution using phenolphthalein as the indicator. Dipotassium hydrogen phosphate (secondary or dibasic potassium phosphate), K₂HPO₄, is an amorphous white salt, which is readily soluble in water.

Trisodium phosphate, tertiary sodium phosphate, Na₃PO₄, are colorless crystals with a density of 1.62 gcm⁻³ and a melting point of 73-76° C. (decomposition) as the dodecahydrate; as the decahydrate (corresponding to 19-20% P₂O₅) a melting point of 100° C., and in anhydrous form (corresponding to 39-40% P₂O₅) a density of 2.536 gcm⁻³. Trisodium phosphate is readily soluble in water with an alkaline reaction and is manufactured by evaporating a solution of exactly 1 mole disodium phosphate and 1 mole NaOH. Tripotassium phosphate (tertiary or tribasic potassium phosphate), K₃PO₄, is a white deliquescent granular powder with a density of 2.56 gcm⁻³, has a melting point of 1340° C. and is readily soluble in water through an alkaline reaction. It is produced by e.g. heating Thomas slag with carbon and potassium sulfate. Despite their higher price, the more readily soluble and therefore highly effective potassium phosphates are often preferred to corresponding sodium compounds in the detergent industry.

Tetrasodium diphosphate (sodium pyrophosphate), Na₄P₂O₇, exists in anhydrous form (density 2.534 gcm⁻³, melting point 988° C., a figure of 880° C. has also been mentioned) and as the decahydrate (density 1.815-1.836 gcm⁻³, melting point 94° C. with loss of water). Both substances are colorless crystals that dissolve in water with an alkaline reaction. Na₄P₂O₇ is formed when disodium phosphate is heated to more than 200° C. or by reacting phosphoric acid with soda in a stoichiometric ratio and spray drying the solution. The decahydrate complexes heavy metal salts and hardness salts and, hence, reduces the hardness of water. Potassium diphosphate (potassium pyrophosphate), K₄P₂O₇, exists in the form of the trihydrate and is a colorless hygroscopic powder with a density of 2.33 gcm⁻³, which is soluble in water, the pH of a 1% solution at 25° C. being 10.4.

Relatively high molecular weight sodium and potassium phosphates are formed by condensation of NaH₂PO₄ or KH₂PO₄. They may be divided into cyclic types, namely the sodium and potassium metaphosphates, and chain types, the sodium and potassium polyphosphates. The chain types in particular are known by various different names: fused or calcined phosphates, Graham's salt, Kurrol's salt and Maddrell's salt. All higher sodium and potassium phosphates are known collectively as condensed phosphates.

The industrially important pentasodium triphosphate, Na₅P₃0₁₀ (sodium tripolyphosphate), is anhydrous or crystallizes with 6H₂O to a non-hygroscopic white water-soluble salt which and which has the general formula NaO—[P(O)(ONa)—O]ₙ—Na where n=3. Around 17 g of the salt free from water of crystallization dissolve in 100 g of water at room temperature, around 20 g at 60° C. and around 32 g at 100° C. After heating the solution for 2 hours to 100° C., around 8% orthophosphate and 15% diphosphate are formed by hydrolysis. In the preparation of pentasodium triphosphate, phosphoric acid is reacted with soda solution or sodium hydroxide in a stoichiometric ratio and the solution is spray-dried. Similarly to Graham's salt and sodium diphosphate, pentasodium triphosphate solubilizes many insoluble metal compounds (including lime soaps, etc.). K₅P₃O₁₀ (potassium tripolyphosphate), is marketed for example in the form of a 50% by weight solution (>23% P₂O₅, 25% K₂O). The potassium polyphosphates are widely used in the detergent industry. Sodium potassium tripolyphosphates also exist and are also usable in the scope of the present invention. They are formed for example when sodium trimetaphosphate is hydrolyzed with KOH:
(NaPO₃)₃+2 KOH→Na₃K₂P₃O₁₀+H₂O

According to the invention, they may be used in exactly the same way as sodium tripolyphosphate, potassium tripolyphosphate or mixtures thereof. Mixtures of sodium tripolyphosphate and sodium potassium tripolyphosphate or mixtures of potassium tripolyphosphate and sodium potassium tripolyphosphate or mixtures of sodium tripolyphosphate and potassium tripolyphosphate and sodium potassium tripolyphosphate may also be used in accordance with the invention.

Organic co-builders, which may be used in the washing and cleaning agents according to the invention, include, in particular, polycarboxylates or polycarboxylic acids, polymeric polycarboxylates, polyaspartic acid, polyacetals, optionally oxidized dextrins, other organic co builders (see below) and phosphonates. These classes of substances are described below.

Useful organic builders are, for example, the polycarboxylic acids usable in the form of their sodium salts, polycarboxylic acids in this context being understood to be carboxylic acids that carry more than one acid function. These include, for example, citric acid, adipic acid, succinic acid, glutaric acid, malic acid, tartaric acid, maleic acid, fumaric acid, sugar acids, aminocarboxylic acids, nitrilotriacetic acid (NTA), providing its use is not ecologically unsafe, and mixtures thereof. Preferred salts are the salts of polycarboxylic acids such as citric acid, adipic acid, succinic acid, glutaric acid, tartaric acid, sugar acids and mixtures thereof.

Acids per se can also be used. Besides their building effect, the acids also typically have the property of an acidifying component and, hence also serve to establish a relatively low and mild pH in washing or cleaning agents, when the pH, which results from the mixture of other components, is not wanted. Acids that are system-compatible and environmentally compatible such as citric acid, acetic acid, tartaric acid, malic acid, glycolic acid, succinic acid, glutaric acid, adipic acid, gluconic acid and mixtures thereof are particularly mentioned in this regard. However, mineral acids, particularly sulfuric acid or bases, particularly ammonium or alkali metal hydroxides can also serve as pH regulators. These types of regulators are preferably comprised in the inventive agents in amounts of not more than 20 wt. %, particularly from 1.2 wt. % to 17 wt. %.

Other suitable builders are polymeric polycarboxylates, i.e. for example the alkali metal salts of polyacrylic or polymethacrylic acid, for example those with a relative molecular weight of 500 to 70 000 g/mol.

The molecular weights mentioned in this specification for polymeric polycarboxylates are weight-average molecular weights M_{w} of the particular acid form which, fundamentally, were determined by gel permeation chromatography (GPC), equipped with a UV detector. The measurement was carried out against an external polyacrylic acid standard, which provides realistic molecular weight values by virtue of its structural similarity to the polymers investigated. These values differ significantly from the molecular weights measured against polystyrene sulfonic acids as standard. The molecular weights measured against polystyrene sulfonic acids are generally significantly higher than the molecular weights mentioned in this specification.

Particularly suitable polymers are polyacrylates, which preferably have a molecular weight of 2000 to 20 000 g/mol. By virtue of their superior solubility, preferred representatives of this group are the short-chain polyacrylates, which have molecular weights of 2000 to 10 000 g/mol and, more particularly, 3000 to 5000 g/mol.

Further suitable copolymeric polycarboxylates are particularly those of acrylic acid with methacrylic acid and of acrylic acid or methacrylic acid with maleic acid. Copolymers of acrylic acid with maleic acid, which comprise 50 to 90 wt. % acrylic acid and 50 to 10 wt. % maleic acid, have proven to be particularly suitable. Their relative molecular weight, based on free acids, generally ranges from 2000 to 70 000 g/mol, preferably 20 000 to 50 000 g/mol and especially 30 000 to 40 000 g/mol. The (co)polymeric polycarboxylates can be used either as powders or as aqueous solutions. The (co)polymeric polycarboxylate content of the compositions is preferably from 0.5 to 20% by weight, in particular from 1 to 10% by weight.

In order to improve the water solubility, the polymers can also comprise allylsulfonic acids as monomers, such as for example, allyloxybenzenesulfonic acid and methallylsulfonic acid.

Other particularly preferred polymers are biodegradable polymers of more than two different monomer units, for example those which contain salts of acrylic acid and maleic acid and vinyl alcohol or vinyl alcohol derivatives as monomers or those which contain salts of acrylic acid and 2-alkylallyl sulfonic acid and sugar derivatives as monomers.

Other preferred copolymers are those, which preferably contain acrolein and acrylic acid/acrylic acid salts or acrolein and vinyl acetate as monomers.

Similarly, other preferred builders are polymeric aminodicarboxylic acids, salts or precursors thereof. Polyaspartic acids or their salts and derivatives are particularly preferred.

Further preferred builders are polyacetals that can be obtained by treating dialdehydes with polyol carboxylic acids that possess 5 to 7 carbon atoms and at least 3 hydroxyl groups. Preferred polyacetals are obtained from dialdehydes like glyoxal, glutaraldehyde, terephthalaldehyde as well as their mixtures and from polycarboxylic acids like gluconic acid and/or glucoheptonic acid.

Further suitable organic builders are dextrins, for example oligomers or polymers of carbohydrates that can be obtained by the partial hydrolysis of starches. The hydrolysis can be carried out using typical processes, for example acidic or enzymatic catalyzed processes. The hydrolysis products preferably have average molecular weights in the range 400 to 500 000 g/mol. A polysaccharide with a dextrose equivalent (DE) of 0.5 to 40 and, more particularly, 2 to 30 is preferred, the DE being an accepted measure of the reducing effect of a polysaccharide by comparison with dextrose, which has a DE of 100. Both maltodextrins with a DE between 3 and 20 and dry glucose syrups with a DE between 20 and 37 and also so-called yellow dextrins and white dextrins with relatively high molecular weights of 2000 to 30 000 g/mol may be used.

The oxidized derivatives of such dextrins concern their reaction products with oxidizing agents that are capable of oxidizing at least one alcohol function of the saccharide ring to the carboxylic acid function. Particularly preferred organic builders for inventive compositions are oxidized starches and their derivatives from the applications EP 472 042, WO 97/25399, and EP 755 944.

Oxydisuccinates and other derivatives of disuccinates, preferably ethylenediamine disuccinate are also further suitable cobuilders. Ethylenediamine-N,N′-disuccinate (EDDS) is preferably used here in the form of its sodium or magnesium salts. In this context, glycerine disuccinates and glycerine trisuccinates are also preferred. Suitable addition quantities in zeolite-containing and/or silicate-containing formulations range from 3 to 15% by weight.

Other useful organic co-builders are, for example, acetylated hydroxycarboxylic acids and salts thereof which may optionally be present in lactone form and which contain at least 4 carbon atoms, at least one hydroxy group and at most two acid groups.

The phosphonates represent a further class of substances with cobuilder properties. In particular, they are hydroxyalkane phosphonates or aminoalkane phosphonates. Among the hydroxyalkane phosphonates, 1-hydroxyethane-1,1-diphosphonate (HEDP) is of particular importance as the cobuilder. It is normally added as the sodium salt, the disodium salt reacting neutral and the tetrasodium salt reacting alkaline (pH 9). Ethylenediamine tetramethylene phosphonate (EDTMP), diethylenetriamine pentamethylene phosphonate (DTPMP) and their higher homologs are preferably chosen as aminoalkane phosphonates. They are preferably added in the form of the neutral-reacting sodium salts, e.g. as the hexasodium salt of EDTMP or as the hepta and octasodium salt of DTPMP. Of the phosphonates, HEDP is preferably used as the builder. The aminoalkane phosphonates additionally possess a pronounced ability to complex heavy metals. Accordingly, it can be preferred, particularly where the agents also contain bleach, to use aminoalkane phosphonates, particularly DTPMP, or mixtures of the mentioned phosphonates.

In addition, any compounds capable of forming complexes with alkaline earth metal ions may be used as co-builders.

Builders can be comprised in the inventive compositions optionally in quantities of up to 90% by weight. They are preferably comprised in quantities of up to 75% by weight. Inventive detergents possess builder contents of particularly 5 wt. % to 50 wt. %. In inventive compositions for cleaning hard surfaces, in particular for automatic dishwashing, the content of builders is particularly 5 wt. % to 88 wt. %, wherein in this type of composition, no water-insoluble builders are employed. In a preferred embodiment, the inventive composition, particularly for automatic dishwashers, comprises 20 wt. % to 40 wt. % of water-soluble organic builders, particularly alkali citrate, 5 wt. % to 15 wt. % alkali carbonate and 20 wt. % to 40 wt. % alkali disilicate.

Solvents that can be added to the liquid to gel-like compositions of washing and cleaning agents originate, for example, from the group of mono- or polyhydric alcohols, alkanolamines or glycol ethers, in so far that they are miscible with water in the defined concentrations. Preferably, the solvents are selected from ethanol, n- or i-propanol, butanols, ethylene glycol methyl ether, ethylene glycol ethyl ether, ethylene glycol propyl ether, ethylene glycol mono-n-butyl ether, diethylene glycol methyl ether, diethylene glycol ethyl ether, propylene glycol methyl-, -ethyl- or -propyl ether, dipropylene glycol methyl-, or -ethyl ether, methoxy-, ethoxy- or butoxy triglycol, 1-butoxyethoxy-2-propanol, 3-methyl-3-methoxybutanol, propylene glycol t-butyl ether as well as mixtures of these solvents.

Solvents can be employed in the inventive liquid to gel-like detergents and cleaning compositions in amounts between 0.1 and 20 wt. %, preferably, however below 15 wt. % and particularly below 10 wt. %.

One or more thickeners or thickener systems can be added to the inventive compositions to adjust the viscosity. These high molecular weight substances, which are also called swelling agents, soak up mostly liquids, thereby swelling up and subsequently transform into viscous, real or colloidal solutions.

Suitable thickeners are inorganic or polymeric organic compounds. The inorganic thickeners include, for example, polysilicic acids, mineral clays like montmorillonite, zeolites, silicic acids and bentonites. The organic thickeners come from the groups of natural polymers, derivatives of natural polymers and synthetic polymers. Exemplary, naturally occurring polymers that can be used as thickeners are agar agar, carrageen, tragacanth, gum Arabic, alginates, pectins, polyoses, guar meal, locust tree bean flour, starches, dextrins, gelatines and casein. Modified natural products that are used as thickeners are mainly derived from the group of the modified starches and celluloses. Examples can be cited as carboxymethyl cellulose and other cellulose ethers, hydroxyethyl- and hydroxypropyl cellulose as well as flour ether. Totally synthetic thickeners are polymers such as polyacrylics and polymethacrylics, vinyl polymers, polycarboxylic acids, polyethers, polyimines, polyamides and polyurethanes.

The thickeners can be comprised in amounts up to 5 wt. %, preferably from 0.05 to 2 wt. %, and particularly preferably from 0.1 to 1.5 wt. %, based on the finished preparation.

The detergents or cleaning agents according to the invention can optionally comprise other conventional ingredients, such as sequestering agents, electrolytes and further auxiliaries.

The washing agents for textiles may contain derivatives of diaminostilbene disulfonic acid or alkali metal salts thereof as optical brighteners. Suitable optical brighteners are, for example, salts of 4,4′-bis-(2-anilino-4-morpholino-1,3,5-triazinyl-6-amino)stilbene-2,2′-disulfonic acid or compounds of similar structure which contain a diethanolamino group, a methylamino group, an anilino group or a 2-methoxyethylamino group instead of the morpholino group. Optical brighteners of the substituted diphenylstyryl type may also be present, for example the alkali metal salts of 4,4′-bis(2-sulfostyryl)diphenyl, 4,4′-bis(4-chloro-3-sulfostyryl)diphenyl or 4-(4-chlorostyryl)-4′-(2-sulfostyryl)diphenyl. Mixtures of the mentioned optical brighteners may also be used.

Graying inhibitors have the task of ensuring that the dirt removed from the textile fibers is held suspended in the wash liquid. Water-soluble colloids of mostly organic nature are suitable for this, for example starch, glue, gelatines, salts of ether carboxylic acids or ether sulfonic acids of starches or celluloses, or salts of acidic sulfuric acid esters of celluloses or starches. Water-soluble, acid group-containing polyamides are also suitable for this purpose. Moreover, aldehyde starches, for example, can be used instead of the abovementioned starch derivatives. Preference, however, is given to the use of cellulose ethers such as carboxymethyl cellulose (Na salt), methyl cellulose, hydroxyalkyl cellulose, and mixed ethers such as methyl hydroxyethyl cellulose, methyl hydroxypropyl cellulose, methyl carboxymethyl cellulose and mixtures thereof, which can be added, for example in amounts of 0.1 to 5 wt. %, based on the agent.

In order to realize a silver corrosion protection, silver protectors for tableware can be added to the inventive cleaning agents. Benzotriazoles, ferric chloride or CoSO₄, for example are known from the prior art. As is known from the European Patent EP 0 736 084 B1, for example, particularly suitable silver corrosion inhibitors for general use with enzymes, are salts and/or complexes of manganese, titanium, zirconium, hafnium, vanadium, cobalt or cerium, in which the cited metals exist in the valence states II, III, IV, V or VI. Examples of these types of compounds are MnSO₄, V₂O₅, V₂O₄, VO₂, TiOSO₄, K₂TiF₆, K₂ZrF₆, Co(NO₃)₂, Co(NO₃)₃ and mixtures thereof.

Soil repellents are mostly polymers that when used in a detergent lend the fibers soil repelling properties and/or support the soil repellent capabilities of the conventional ingredients. A comparable effect can also be observed when they are added in cleaning compositions for hard surfaces.

Particularly effective and well-known soil release agents are copolyesters with dicarboxylic acid, alkylene glycol and polyalkylene glycol units. Examples of these are copolymers or mixed polymers of polyethylene terephthalates and polyoxyethylene glycol (DT 16 17 141 and DT 22 00 911). German Offenlegungsschrift DT 22 53 063 cites acid compositions, which inter alia comprise a copolymer of a dibasic acid and an alkylene or cycloalkylene polyglycol. Polymers of ethylene terephthalate and polyethylene oxide terephthalate and their use in detergents are described in the German texts DE 28 57 292 and DE 33 24 258 and the European Patent EP 0 253 567. The European Patent EP 066 944 relates to agents, which contain a copolyester of ethylene glycol, polyethylene glycol, aromatic dicarboxylic acids and sulfonated aromatic dicarboxylic acids in defined molar ratios. Polyesters, end-capped with methyl or ethyl groups, with ethylene and/or propylene terephthalate units and polyethylene oxide terephthalate units and detergents that comprise such a soil-release polymer are known from EP 0 185 427. The European Patent EP 0 241 984 relates to a polyester, which beside oxyethylene groups and terephthalic acid units also comprises substituted ethylene units as well as glycerine units. Polyesters are known from EP 0 241 985 which contain, beside oxyethylene groups and terephthalic acid units, 1,2-propylene, 1,2-butylene and/or 3-methoxy-1,2-propylene groups as well as glycerine units, and are end-capped with C₁ to C₄ alkyl groups. Polyesters with polypropylene terephthalate units and polyoxyethylene terephthalate units, at least partially end-capped with C₁₋₄ alkyl or acyl groups, are known from the European Patent application EP 0 272 033. The European Patent EP 0 274 907 describes soil-release polyesters containing terephthalate end-capped with sulfoethyl groups. According to the European Patent application EP 0 357 280, soil-release polyesters with terephthalate units, alkylene glycol units and poly-C₂₋₄ glycol units are manufactured by sulfonation of the unsaturated end groups. The international patent application WO 95/32232 relates to acidic, aromatic polyesters that are capable of releasing soil. Non-polymeric soil repellent active substances for cotton materials with a plurality of functional units are known from the international patent application WO 97/31085: a first unit, which can be cationic, for example, is able to be adsorbed onto the cotton surface by electrostatic attraction, and a second unit, which is designed to be hydrophobic, is responsible for the retention of the active agent at the water/cotton interface.

Color transfer inhibitors that can be used in inventive detergents for textiles particularly include polyvinyl pyrrolidones, polyvinyl imidazoles, polymeric N-oxides such as polyvinyl pyridine-N-oxide and copolymers of vinyl pyrrolidone with vinyl imidazole.

On using the agents in automatic cleaning processes, it can be advantageous to add foam inhibitors. Suitable foam inhibitors include for example, soaps of natural or synthetic origin, which have a high content of C₁₈-C₂₄ fatty acids. Suitable non-surface-active types of foam inhibitors are, for example, organopolysiloxanes and mixtures thereof with microfine, optionally silanized silica and also paraffins, waxes, microcrystalline waxes and mixtures thereof with silanized silica or bis-stearyl ethylene diamide. Mixtures of various foam inhibitors, for example mixtures of silicones, paraffins or waxes, are also used with advantage. Preferably, the foam inhibitors, especially silicone-containing and/or paraffin-containing foam inhibitors, are loaded onto a granular, water-soluble or dispersible carrier material. Especially in this case, mixtures of paraffins and bis stearyl ethylene diamides are preferred.

An inventive cleaning composition for hard surfaces can moreover comprise abrasive ingredients, especially from the group comprising quartz meal, wood flour, plastic powder, chalk and microspheres as well as their mixtures. Abrasives are preferably comprised in the inventive cleaning compositions in amounts of not more than 20 wt. %, particularly from 5 wt. % to 15 wt. %.

Colorants and fragrances may be added to the detergents or cleaning agents in order to improve the aesthetic impression created by the products and to provide the consumer not only with the required washing and cleaning performance but also with a visually and sensorially “typical and unmistakable” product. Suitable perfume oils or fragrances include individual perfume compounds, for example synthetic products of the ester, ether, aldehyde, ketone, alcohol and hydrocarbon type. Perfume compounds of the ester type are, for example, benzyl acetate, phenoxyethyl isobutyrate, p-tert.-butylcyclohexyl acetate, linalyl acetate, dimethylbenzyl carbinyl acetate, phenylethyl acetate, linalyl benzoate, benzyl formate, ethylmethylphenyl glycinate, allylcyclohexyl propionate, styrallyl propionate and benzyl salicylate. The ethers include, for example, benzyl ethyl ether; the aldehydes include, for example, the linear alkanals containing 8 to 18 carbon atoms, citral, citronellal, citronellyloxyacetaldehyde, cyclamen aldehyde, hydroxycitronellal, lilial and bourgeonal; the ketones include, for example, the ionones, α-isomethyl ionone and methyl cedryl ketone; the alcohols include anethol, citronellol, eugenol, geraniol, linalool, phenylethyl alcohol and terpineol and the hydrocarbons include, above all, the terpenes, such as limonene and pinene. However, mixtures of various odoriferous substances, which together produce an attractive perfume note, are preferably used. Perfume oils such as these may also contain natural odoriferous mixtures obtainable from vegetal sources, for example pine, citrus, jasmine, patchouli, rose or ylang-ylang oil. Also suitable are muscatel oil, oil of sage, chamomile oil, clove oil, melissa oil, mint oil, cinnamon leaf oil, lime blossom oil, juniper berry oil, vetivert oil, olibanum oil, galbanum oil and laudanum oil and orange blossom oil, neroli oil, orange peel oil and sandalwood oil. Normally the content of dyes lies below 0.01 wt. %, while fragrances can make up to 2 wt. % of the total formulation of the detergents and cleaning composition.

The fragrances may be directly incorporated in the washing and cleaning agents, although it can also be of advantage to apply the fragrances on carriers, which reinforce the adsorption of the perfume on the washing and thereby ensuring a long-lasting fragrance on the textiles by decreasing the release of the fragrance, especially for treated textiles. Suitable carrier materials are, for example, cyclodextrins, the cyclodextrin/perfume complexes optionally being coated with other auxiliaries. A further preferred carrier for fragrances is the described zeolite X, which instead of or in mixtures with surfactants can also take up fragrances. Accordingly, preferred detergents and cleaning agents comprise the described zeolite X and fragrances that are preferably at least partially absorbed on the zeolite.

Preferred colorants, which are not difficult for the expert to choose, have high storage stability, are not affected by the other ingredients of the detergents or by light and do not have any pronounced substantivity for the textile fibers being treated, so as not to color them.

To control microorganisms, the washing or cleaning agents may contain antimicrobial agents. Depending on the antimicrobial spectrum and the action mechanism, antimicrobial agents are classified as bacteriostatic agents and bactericides, fungistatic agents and fungicides, etc. Important substances from these groups are for example benzalkonium chlorides, alkylaryl sulfonates, halophenols and phenol mercury acetate. In the present context of the inventive teaching, the expressions “antimicrobial activity” and “antimicrobial agent” have the usual technical meanings as defined, for example, by K. H. Wallhäuβer in “Praxis der Sterilisation, Desinfektion—Konservierung Keimidentifizierung—Betriebshygiene” (5th Edition, Stuttgart/New York: Thieme, 1995), any of the substances with antimicrobial activity described therein being usable.

Suitable antimicrobial agents are preferably selected from the groups of alcohols, amines, aldehydes, antimicrobial acids and salts thereof, carboxylic acid esters, acid amides, phenols, phenol derivatives, diphenyls, diphenylalkanes, urea derivatives, oxygen and nitrogen acetals and formals, benzamidines, isothiazolines, phthalimide derivatives, pyridine derivatives, antimicrobial surface-active compounds, guanidines, antimicrobial amphoteric compounds, quinolines, 1,2-dibromo-2,4-dicyanobutane, iodo-2-propyl butyl carbamate, iodine, iodophores, peroxy compounds, halogen compounds and mixtures of the above.

Consequently, the antimicrobial active substances can be chosen among ethanol, n-propanol, i-propanol, 1,3-butanediol, phenoxyethanol, 1,2-propylenelycol, glycerin, undecylenic acid, benzoic acid, salicylic acid, dihydracetic acid, o-phenylphenol, N-methylmorpholine-acetonitrile (MMA), 2-benzyl-4-chlorophenol, 2,2′-methylene-bis-(6-bromo-4-chlorophenol), 4,4′-dichloro-2′-hydroxydiphenyl ether (dichlosan), 2,4,4′-trichloro-2′-hydroxydiphenyl ether (trichlosan), chlorhexidine, N-(4-chlorophenyl)-N-(3,4-dichlorophenyl)-urea, N,N′-(1,10-decanediyldi-1-pyridinyl-4-ylidene)-bis-(1-octamine)dihydrochloride, N,N′-bis-(4-chlorophenyl)-3,12-diimino-2,4,11,13-tetraaza-tetradecanediimide amide, glucoprotamines, surface-active antimicrobial quaternary compounds, guanidines, including the bi- and polyguanidines, such as for example 1,6-bis(2-ethylhexylbiguanidohexane)dihydrochloride, 1,6-di-(N₁,N₁′-phenyldiguanido-N₅,N₅′)hexane tetrahydrochloride, 1,6-di-(N1,N1-phenyl-N₁,N₁-methyldiguanido-N₅,N₅′)hexane dihydrochloride, 1,6-di-(N₁,N₁′-o-chlorophenyldiguanido-N₅,N₅′)hexane dihydrochloride, 1,6-di-(N₁,N₁′-2,6-dichlorophenyldiguanido-N₅,N₅′)hexane dihydrochloride, 1,6-di-[N₁,N₁′-β-(p-methoxyphenyl)diguanido-N₅,N₅′]hexane dihydrochloride, 1,6-di-(N₁,N₁′-α-methyl-β-phenyldiguanido-N₅,N₅′)hexane dihydrochloride, 1,6-di-(N₁,N₁′-p-nitrophenyldiguanido-N₅,N₅′)hexane dihydrochloride, ω:ω-di-(N₁,N₁′-phenyldiguanido-N5,N5′)di-n-propyl ether dihydrochloride, ω:ω-di-(N₁,N₁′-p-chlorophenyldiguanido-N₅,N₅′)di-n-propyl ether tetrahydrochloride, 1,6-di-(N₁,N₁′-2,4-dichlorophenyldiguanido-N₅,N₅′)hexane tetrahydrochloride, 1,6-di-(N₁,N₁′-p-methylphenyldiguanido-N₅,N₅′)hexane dihydrochloride, 1,6-di-(N₁,N₁′-2,4,5-trichlorophenyldiguanido-N₅,N₅′)hexane tetrahydrochloride, 1,6-di-[N₁,N₁′-α-(p-chlorophenyl)ethyldiguanido-N₅,N₅′]hexane dihydrochloride, ω:ω-di-(N₁,N₁′-p-chlorophenyldiguanido-N₅,N₅′)m-xylene dihydrochloride, 1,12-di-(N₁,N₁′-p-chlorophenyldiguanido-N₅,N₅′)dodecane dihydrochloride, 1,10-di-(N₁,N₁′-phenyldiguanido-N₅,N₅′)decane tetrahydrochloride, 1,12-di-(N₁,N₁′-phenyldiguanido-N₅,N₅′)dodecane tetrahydrochloride, 1,6-di-(N₁,N₁′-o-chlorophenyldiguanido-N₅,N₅′)hexane dihydrochloride, 1,6-di-(N₁,N₁′-o-chlorophenyldiguanido-N₅,N₅′)hexane tetrahydrochloride, ethylene-bis-(1-tolylphenylbiguanide), ethylene-bis-(p-tolylphenylbiguanide), ethylene-bis-(3,5-dimethylphenylbiguanide), ethylene-bis-(p-tert-amylphenylbiguanide), ethylene-bis-(nonylphenylbiguanide), ethylene-bis-(phenylbiguanide), ethylene-bis-(N-butylphenylbiguanide), ethylene-bis-(2,5-diethoxyphenylbiguanide), ethylene-bis-(2,4-dimethylphenylbiguanide), ethylene-bis-(o-diphenylbiguanide), ethylene-bis-(mixed amylnaphthylbiguanide), N-butylethylene-bis-(phenylbiguanide), trimethylene bis(o-tolylbiguanide), N-butyltrimethylene-bis-(phenylbiguanide) and the corresponding salts like acetates, gluconates, hydrochlorides, hydrobromides, citrates, bisulfites, fluorides, polymaleates, N-coco alkyl sarcinosates, phosphites, hypophosphites, perfluorooctanoates, silicates, sorbates, salicylates, maleates, tartrates, fumarates, ethylenediaminetetraacetates, iminodiacetates, cinnamates, thiocyanates, arginates, pyromellitates, tetracarboxybutyrates, benzoates, glutarates, monofluorophosphates, perfluoropropionates as well as any mixtures thereof. Furthermore, halogenated xylene- and cresol derivatives are suitable, such as p-chloro-meta-cresol, p-chloro-meta-xylene, as well as natural antimicrobial active agents of plant origin (e.g. from spices or aromatics), animal as well as microbial origin. Preferred antimicrobial agents are antimicrobial surface-active quaternary compounds, a natural antimicrobial agent of vegetal origin and/or a natural antimicrobial agent of animal origin and, most preferably, at least one natural antimicrobial agent of vegetal origin from the group comprising caffeine, theobromine and theophylline and essential oils, such as eugenol, thymol and geraniol, and/or at least one natural antimicrobial agent of animal origin from the group comprising enzymes, such as protein from milk, lysozyme and lactoperoxidase and/or at least one antimicrobial surface-active quaternary compound containing an ammonium, sulfonium, phosphonium, iodonium or arsonium group, peroxy compounds and chlorine compounds. Substances of microbial origin, so-called bacteriozines, may also be used.

The quaternary ammonium compounds (QUATS) suitable as antimicrobial agents have the general formula (R¹)(R²)(R³)(R⁴)N⁺X⁻, in which R¹ to R⁴ may be the same or different and represent C₁₋₂₂ alkyl groups, C₇₋₂₈ aralkyl groups or heterocyclic groups, two or—in the case of an aromatic compound, such as pyridine—even three groups together with the nitrogen atom forming the heterocycle, for example a pyridinium or imidazolinium compound, and X⁻¹ represents halide ions, sulfate ions, hydroxide ions or similar anions. In the interests of optimal antimicrobial activity, at least one of the substituents preferably has a chain length of 8 to 18 and, more preferably, 12 to 16 carbon atoms.

QUATS can be obtained by reacting tertiary amines with alkylating agents such as, for example, methyl chloride, benzyl chloride, dimethyl sulfate, dodecyl bromide and also ethylene oxide. The alkylation of tertiary amines having one long alkyl chain and two methyl groups is particularly easy. The quaternization of tertiary amines containing two long chains and one methyl group can also be carried out under mild conditions using methyl chloride. Amines containing three long alkyl chains or hydroxy-substituted alkyl chains lack reactivity and are preferably quaternized with dimethyl sulfate.

Suitable QUATS are, for example, benzalkonium chloride (N-alkyl-N,N-dimethylbenzyl ammonium chloride, CAS No. 8001-54-5), benzalkon B (m,p-dichlorobenzyl dimethyl-C₁₂-alkyl ammonium chloride, CAS No. 58390-78-6), benzoxonium chloride (benzyldodecyl-bis-(2-hydroxyethyl) ammonium chloride), cetrimonium bromide (N-hexadecyl-N,N-trimethyl ammonium bromide, CAS No. 57-09-0), benzetonium chloride (N,N-di-methyl-N-[2-[2-[p-(1,1,3,3-tetramethylbutyl)-phenoxy]-ethoxy]-ethyl]-benzyl ammonium chloride, CAS No. 121-54-0), dialkyl dimethyl ammonium chlorides, such as di-n-decyldimethyl ammonium chloride (CAS No. 7173-51-5-5), didecyldimethyl ammonium bromide (CAS No. 2390-68-3), dioctyl dimethyl ammonium chloride, 1-cetylpyridinium chloride (CAS No. 123-03-5) and thiazoline iodide (CAS No. 1576448-1) and mixtures thereof. Particularly preferred QUATS are the benzalkonium chlorides containing C₈₋₁₈ alkyl groups, more particularly C₁₂₋₁₄ alkyl benzyl dimethyl ammonium chloride.

Benzalkonium halides and/or substituted benzalkonium halides are commercially obtainable, for example, as Barquat® from Lonza, Marquato® from Mason, Variquat® from Witco/Sherex and Hyamine® from Lonza and as Bardac® from Lonza. Other commercially obtainable antimicrobial agents are N-(3-chloroallyl)-hexaminium chloride, such as Dowicide® and Dowicil® from Dow, benzethonium chloride, such as Hyamine® 1622 from Rohm & Haas, methyl benzethonium chloride, such as Hyamine® 10X from Rohm & Haas, cetyl pyridinium chloride, such as cepacolchloride from Merrell Labs.

The antimicrobial agents are used in quantities of 0.0001% by weight to 1% by weight, preferably 0.001% by weight to 0.8% by weight, particularly preferably 0.005% by weight to 0.3% by weight and most preferably 0.01 to 0.2% by weight.

The compositions may also comprise UV absorbers, which attach to the treated textiles and improve the light stability of the fibers and/or the light stability of the various ingredients of the formulation. UV-absorbers are understood to mean organic compounds, which are able to absorb UV radiation and emit the resulting energy in the form of longer wavelength radiation, for example as heat.

Compounds, which possess these desired properties, are for example, the efficient radiationless deactivating derivatives of benzophenone having substituents in position(s) 2 and/or 4. Also suitable are substituted benzotriazoles, acrylates, which are phenyl-substituted in position 3 (cinnamic acid derivatives), with or without cyano groups in position 2, salicylates, organic Ni complexes, as well as natural substances such as umbelliferone and the endogenous urocanic acid. The biphenyl and above all the stilbene derivatives such as for example those described in EP 0728749 A and commercially available as Tinosorb® FD or Tinosorb® FR from Ciba, are of particular importance. As UV-B absorbers can be cited: 3-benzylidenecamphor or 3-benzylidenenorcamphor and its derivatives, for example 3-(4-methylbenzylidene)camphor, as described in the EP 0693471 B1; 4-aminobenzoic acid derivatives, preferably 4-(dimethylamino)benzoic acid, 2-ethylhexyl ester, 4-(dimethylamino)benzoic acid, 2-octyl ester and 4-(dimethylamino)benzoic acid, amylester; esters of cinnamic acid, preferably 4-methoxycinnamic acid, 2-ethylhexyl ester, 4-methoxycinnamic acid, propyl ester, 4-methoxycinnamic acid, isoamyl ester, 2-cyano-3,3-phenylcinnamic acid, 2-ethylhexyl ester (octocrylene); esters of salicylic acid, preferably salicylic acid, 2-ethylhexyl ester, salicylic acid, 4-isopropylbenzyl ester, salicylic acid, homomenthylester; derivatives of benzophenone, preferably 2-hydroxy-4-methoxybenzophenone, 2-hydroxy-4-methoxy-4′-methylbenzophenone, 2,2′-dihydroxy-4-methoxybenzophenone; esters of benzalmalonic acid, preferably 4-methoxybenzmalonic acid, di-2-ethylhexylester; triazine derivatives, such as, for example 2,4,6-trianilino-(p-carbo-2′-ethyl-1′-hexyloxy)-1,3,5-triazine and octyl triazone, as described in EP 0818450 A1 or dioctyl butamidotriazone (Uvasorb® HEB); propane-1,3-dione, such as for example 1-(4-tert. butylphenyl)-3-(4′-methoxyphenyl)propane-1,3-dione; ketotricyclo(5.2.1.0) decane derivatives, as described in EP 0694521 B1. Further suitable are 2-phenylbenzimidazole-5-sulfonic acid and its alkali-, earth alkali-, ammonium-, alkyl ammonium-, alkanol ammonium- and glucammonium salts; sulfonic acid derivatives of benzophenones, preferably 2-hydroxy-4-methoxybenzophenone-5-sulfonic acid and its salts; sulfonic acid derivatives of 3-benzylidenecamphor, as for example 4-(2-oxo-3-bornylidenemethyl)benzene sulfonic acid and 2-methyl-5-(2-oxo-3-bornylidene)sulfonic acid and its salts.

Typical UV-A filters particularly include derivatives of benzoylmethane, such as, for example 1-(4′-tert.-butylphenyl)-3-(4′-methoxyphenyl)propane-1,3-dione, 4-tert.-butyl-4′-methoxydibenzoylmethane (Parsol 1789), 1-phenyl-3-(4′-isopropylphenyl)-propane-1,3-dione as well as enamine compounds, as described in the DE 19712033 A1 (BASF). Naturally, the UV-A and UV-B filters can also be added as mixtures. Beside the cited soluble materials, also insoluble, light protective pigments, namely finely dispersed, preferably, nano metal oxides or salts can be considered for this task. Exemplary suitable metal oxides are particularly zinc oxide and titanium oxide and also oxides of iron, zirconium, silicon, manganese, aluminum and cerium as well as their mixtures. Silicates (talk), barium sulfate or zinc stearate cab be added as salts. The oxides and salts are already used in the form of pigments for skin care and skin protecting emulsions and decorative cosmetics. Here, the particles should have an average diameter of less than 100 nm, preferably between 5 and 50 nm and especially between 15 and 30 nm. They can be spherical, however elliptical or other shaped particles can be used. The pigments can also be surface treated, i.e. hydrophilized or hydrophobized. Typical examples are coated titanium dioxides, such as, for example Titandioxid Z 805 (Degussa) or Eusolex® T2000 (Merck); hydrophobic coating agents preferably include trialkoxy octylsilanes or silicones. Micronized zinc oxide is preferably used. Further suitable UV light protection filters may be found in the review by P. Finkel in SöFW-Journal, volume 122 (1996), p. 543.

The UV absorbers are normally used in amounts of 0.01 wt. % to 5 wt. %, preferably from 0.03 wt. % to 1 wt. %.

To increase their washing or cleaning power, compositions according to the invention can comprise, in addition to the inventive enzymes, additional enzymes, in principle any enzyme established for these purposes in the prior art being useable. These particularly include proteases, amylases, lipases, hemicellulases, cellulases or oxidoreductases as well as preferably their mixtures. In principle, these enzymes are of natural origin; improved variants based on the natural molecules are available for use in detergents and accordingly they are preferred. The detergents according to the invention preferably comprise enzymes in total quantities of 1×10⁻⁶ to 5 weight percent based on active protein. Protein concentrations can be determined using known methods, for example the BCA Process (bicinchoninic acid; 2,2′-bichinolyl-4,4′-dicarboxylic acid) or the biuret process (A. G. Gornall, C. S. Bardawill and M. M. David, J. Biol. Chem., 177 (1948), p. 751-766).

Preferred proteases are those of the subtilisin type. Examples of these are subtilisins BPN′ and Carlsberg, the protease PB92, the subtilisins 147 and 309, the alkaline protease from Bacillus lentus, subtilisin DY and those enzymes of the subtilases no longer however classified in the stricter sense as subtilisins thermitase, proteinase K and the proteases TW3 und TW7. Subtilisin Carlsberg in further developed form is available under the trade name Alcalase® from Novozymes A/S, Bagsvaerd, Denmark. Subtilisins 147 and 309 are commercialized under the trade names Esperase® and Savinase® by the Novozymes company. Variants derived from the protease from Bacillus lentus DSM 5483 (WO 91/02792 A1) are called BLAP®, described particularly in WO 92/21760 A1, WO 95/23221 A1, WO 02/088340 A2 and WO 03/038082 A2. Further suitable proteases from various Bacillus sp. and B. gibsonii emerge from the Patent applications WO 03/054185 A1, WO 03/056017 A2, WO 03/055974 A2 and WO 03/054184 A1.

Further useable proteases are, for example, those enzymes available with the trade names Durazym®, Relase®, Everlase®, Nafizym, Natalase®, Kannase® and Ovozymes® from the Novozymes Company, those under the trade names Purafect®, Purafect® OxP and Properase® from Genencor, that under the trade name Protosol® from Advanced Biochemicals Ltd., Thane, India, that under the trade name Wuxi® from Wuxi Snyder Bioproducts Ltd., China, those under the trade names Proleather® and Protease P® from Amano Pharmaceuticals Ltd., Nagoya, Japan, and that under the designation Proteinase K-16 from Kao Corp., Tokyo, Japan.

Examples of further useable amylases according to the invention are the α-amylases from Bacillus licheniformis, from B. amyloliquefaciens and from B. stearothermophilus, as well as their improved further developments for use in detergents. The enzyme from B. licheniformis is available from the Company Novozymes under the name Termamyl® and from the Genencor Company under the name Purastar®ST. Further development products of this α-amylase are available from the Company Novozymes under the trade names Duramyl® and Termamyl®ultra, from the Company Genencor under the name Purastar®OxAm and from the Company Daiwa Seiko Inc., Tokyo, Japan as Keistase®. The α-amylase from B. amyloliquefaciens is commercialized by the Company Novozymes under the name BAN®, and derived variants from the α-amylase from B. stearothermophilus under the names BSG® and Novamyl®, also from the Company Novozymes.

Moreover, for these purposes, attention should be drawn to the α-amylase from Bacillus sp. A 7-7 (DSM 12368) disclosed in the application WO 02/10356 A2 and the cyclodextrin-glucanotransferase (CGTase) from B. agaradherens (DSM 9948) described in WO 02/44350 A2. Amylolytic enzymes can also be used, which belong to the sequence space of α-amylases, defined in the application WO 03/002711 A2, and those described in the application WO 03/054177 A2. Similarly the fusion products of the cited molecules can be used, for example those in the application DE 10138753 A1.

Moreover, further developments of α-amylase from Aspergillus niger and A. oryzae available from the Company Novozymes under the trade name Fungamyl® are suitable. A further suitable commercial product is, for example Amylase-LT®.

The compositions according to the invention can comprise lipases or cutinases, particularly due to their triglyceride cleaving activities, but also in order to produce in situ peracids from suitable preliminary steps. These include the available or further developed lipases originating from Humicola lanuginosa (Thermomyces lanuginosus), particularly those with the amino acid substitution D96L. They are commercialized, for example by the Novozymes Company under the trade names Lipolase®, Lipolase®Ultra, LipoPrime®, Lipozyme® and Lipex®. Moreover, suitable cutinases, for example are those that were originally isolated from Fusarium solani pisi and Humicola insolens. Likewise useable lipases are available from the Amano Company under the designations Lipase CE®, Lipase P®, Lipase B®, and Lipase CES®, Lipase AKG®, Bacillis sp. Lipase®, Lipase AP®, Lipase M-AP® and Lipase AML®. Suitable lipases or cutinases whose starting enzymes were originally isolated from Pseudomonas mendocina und Fusarium solanii are for example available from Genencor Company. Further important commercial products that may be mentioned are the commercial preparations M1 Lipase® und Lipomax® originally from Gist-Brocades Company, and the commercial enzymes from the Meito Sangyo KK Company, Japan under the names Lipase MY-30®, Lipase OF® and Lipase PL® as well as the product Lumafast® from Genencor Company.

Compositions according to the invention, particularly when they are destined for treating textiles, can comprise cellulases, according to their purpose, as pure enzymes, as enzyme preparations, or in the form of mixtures, in which the individual components advantageously complement their various performances. Among these aspects of performance are particular contributions to primary washing performance, to secondary washing performance of the product, (anti-redeposition activity or inhibition of graying) and softening or brightening (effect on the textile), through to performing a “stone washed” effect.

A usable, fungal endoglucanase(EG)-rich cellulase preparation, or its further developments are offered by the Novozymes Company under the trade name Celluzyme®. The products Endolase® and Carezyme® based on the 50 kD-EG, respectively 43 kD-EG from H. insolens DSM 1800 are also obtainable from Novozymes Company. Further commercial products from this company are Cellusoft® and Renozyme®, based on the application WO 98/12307 A1. Cellulase variants with improved performance emerge from the application WO 98/12307 A1. Similarly, the cellulases disclosed in application WO 97/14804 A1 can be used; for example 20 kD-EG cellulase from Melanocarpus, obtainable from AB Enzymes Company, Finland under the trade names Ecostone® and Biotouch®. Further commercial products from the AB Enzymes Company are Econase® and Ecopulp®. A further suitable cellulase from Bacillus sp. CBS 670.93 is obtainable from the Genencor Company under the trade name Puradax®. Additional commercial products from the Genencor Company are “Genencor detergent cellulase L” and Indiage®Neutra.

Particularly for removing specific problematic stains, the compositions according to the invention can comprise additional enzymes, which are summarized under the term hemicellulases. These include, for example mannanases, xanthanlyases, pectinlyases (=pectinases), pectinesterases, pectatlyases, xyloglucanases (=xylanases), pullulanases and β-glucanases. Suitable mannanases, for example are available under the names Gamanase® and PektinexAR® from Novozymes Company, under the names Rohapec® B1L from AB Enzymes and under the names Pyrolase® from Diversa Corp., San Diego, Calif., USA. A suitable β-Glucanase from a B. alcalophilus emerges from the application WO 99/06573 A1, for example. β-Glucanase extracted from B. subtilis is available under the name Cereflo® from Novozymes Company.

To increase the bleaching action, the detergents or cleaning compositions can comprise oxidoreductases, for example oxidases, oxygenases, katalases, peroxidases, like halo-, chloro-, bromo-, lignin-, glucose- or manganese-peroxidases, dioxygenases or laccases (phenoloxidases, polyphenoloxidases). Suitable commercial products are Denilite® 1 and 2 from the Novozymes Company. Advantageously, additional, preferably organic, particularly preferably aromatic compounds are added that interact with the enzymes to enhance the activity of the relative oxidoreductases (enhancers) or to facilitate the electron flow (mediators) between the oxidizing enzymes and the stains over strongly different redox potentials.

The enzymes used in the inventive compositions either stem originally from microorganisms, such as the species Bacillus, Streptomyces, Humicola, or Pseudomonas, and/or are produced according to known biotechnological processes using suitable microorganisms such as by transgenic expression hosts of the species Bacillus or filamentary fungi.

Purification of the relevant enzymes follows conveniently using established processes such as precipitation, sedimentation, concentration, filtration of the liquid phases, microfiltration, ultrafiltration, mixing with chemicals, deodorization or suitable combinations of these steps.

The enzymes can be added to the inventive compositions in each established form according to the prior art. Included here, for example, are solid preparations obtained by granulation, extrusion or lyophilization, or particularly for liquid agents or agents in the form of gels, enzyme solutions, advantageously highly concentrated, of low moisture content and/or mixed with stabilizers.

Alternatively, all enzymes, both for solid as well as for liquid presentation forms, can be encapsulated, for example by spray drying or extrusion of the enzyme solution together with a preferably natural polymer or in the form of capsules, for example those in which the enzyme is embedded in a solidified gel, or in those of the core-shell type, in which an enzyme-containing core is covered with a water-, air- and/or chemical-impervious protective layer. Further active principles, for example stabilizers, emulsifiers, pigments, bleaches or colorants can be applied in additional layers. Such capsules are made using known methods, for example by vibratory granulation or roll compaction or by fluid bed processes. Advantageously, these types of granulates, for example with an applied polymeric film former are dust-free and as a result of the coating are storage stable.

In addition, it is possible to formulate two or more enzymes together, so that a single granule exhibits a plurality of enzymatic activities.

Protein concentrations on the comprised enzymes, particularly on the comprised choline oxidases can be determined using known methods, for example the BCA Process (bicinchoninic acid; 2,2′-bichinolyl-4,4′-dicarboxylic acid) or the biuret process (A. G. Gornall, C. S. Bardawill and M. M. David, J. Biol. Chem., 177 (1948), p. 751-766).

A protein and/or enzyme in an inventive composition can be protected, particularly in storage, against deterioration such as, for example inactivation, denaturation or decomposition, for example through physical influences, oxidation or proteolytic cleavage. An inhibition of the proteolysis is particularly preferred during microbial preparation of proteins and/or enzymes, particularly when the compositions also contain proteases. According to the invention, stabilizers can be added for this purpose.

One group of stabilizers are reversible protease inhibitors. For this, benzamidine hydrochloride, borax, boric acids, boronic acids or their salts or esters are frequently used, above all derivatives with aromatic groups, for example ortho, meta or para substituted phenyl boronic acids, or their salts or esters. Peptide aldehydes, i.e. oligopeptides with a reduced C-terminus, are also suitable. Ovomucoid and leupeptin, among others, belong to the peptidic reversible protease inhibitors; an additional option is the formation of fusion proteins from proteases and peptide inhibitors.

Further enzyme stabilizers are amino alcohols like mono-, di-, triethanol- and -propanolamine and their mixtures, aliphatic carboxylic acids up to C₁₂, such as for example succinic acid, other dicarboxylic acids or salts of the cited acids. End-capped fatty acid amide alkoxylates are also suitable stabilizers. Specific organic acids, added as builders, are in addition capable of stabilizing a comprised enzyme, as disclosed in WO 97/18287.

Lower aliphatic alcohols, but above all polyols such as, for example glycerol, ethylene glycol, propylene glycol or sorbitol are further frequently used enzyme stabilizers. Di-glycerol phosphate also protects against denaturation by physical influences. Similarly, calcium and/or magnesium salts are used, such as, for example calcium acetate or calcium formate.

Polyamide oligomers or polymeric compounds like lignin, water-soluble vinyl copolymers or cellulose ethers, acrylic polymers and/or polyamides stabilize enzyme preparations against physical influences or pH variations. Polymers containing polyamine-N-oxide act simultaneously as enzyme stabilizers and color transfer inhibitors. Other polymeric stabilizers are linear C₈-C18 polyoxyalkylenes. Alkyl polyglycosides can also stabilize the enzymatic components of the inventive agents and in addition, induce them to increase in performance. Crosslinked nitrogen-containing compounds perform a dual function as soil release agents and as enzyme stabilizers. Hydrophobic, nonionic polymer stabilizes in particular, an optionally comprised cellulase.

Reducing agents and antioxidants increase the stability of enzymes against oxidative decomposition; examples of these are such as sulfur-containing reducing agents. Other examples are sodium sulfite and reducing sugar.

The use of combinations of stabilizers is particularly preferred, for example of polyols, boric acid and/or borax, the combination of boric acid or borate, reducing salts and succinic acid or other dicarboxylic acids or the combination of boric acid or borate with polyols or polyamino compounds and with reducing salts. The effect of peptide-aldehyde stabilizers is conveniently increased by the combination with boric acid and/or boric acid derivatives and polyols and still more by the additional effect of divalent cations, such as for example calcium ions.

In a preferred embodiment, inventive compositions are characterized in that they consist of more than one phase in order to liberate the comprised active principles separately from one another at different times or from different places, for example. This can concern phases in different aggregates, however it particularly concerns phases in the same aggregates.

Inventive compositions, which are composed of more than one solid component, can be easily manufactured by mixing together the various solid components in bulk form, particularly powders, granules or extrudates with various ingredients and/or different release behavior. The manufacture of inventive solid agents with one or more phases can be made by known methods, for example by spray drying or granulation, wherein the enzymes and possible further heat-sensitive ingredients, such as, for example bleaches are optionally added separately. For manufacturing inventive agents with an increased bulk density, particularly in the range of 650 g/l to 950 g/l, a preferred process is one with an extrusion step, known from the European Patent EP 0 486 592. A further preferred manufacturing using a granulation process is described in the European Patent EP 0 642 576.

For solids, proteins can be used, for example, in dried, granulated, encapsulated or encapsulated and additionally dried form. They can be added separately, i.e. as one phase, or together with other ingredients in the same phase, with or without compaction. If microencapsulated, solid enzymes are used, then the water can be removed from the aqueous solutions resulting from the process by means of processes known from the prior art, such as spray-drying, centrifugation or by transdissolution. The particles obtained in this manner normally have a particle size between 50 and 200 μm.

The encapsulated form also serves, as previously discussed, to protect the enzymes from other ingredients such as bleaches, or to enable a controlled release. These capsules are differentiated by size as millicapsules, microcapsules and nanocapsules; microcapsules being particularly preferred for enzymes. Such capsules are disclosed, for example, in the Patent applications WO 97/24177 and DE 199 18 267. Another possible encapsulation method consists in the encapsulation of the enzymes suitable for washing or cleaning agents in starch or in starch derivatives, starting from a mixture of the enzyme solutions with a solution or suspension of starch or a starch derivative. Such an encapsulation process is described in the German application DE 199 56 382.

At least two solid phases can also be combined with each other. Thus, it is possible to prepare a solid composition according to the invention, by compression or compaction into tablets. Such tablets can be monophase or multiphase tablets. Consequently, this presentation form also offers the possibility of displaying a solid inventive composition having two solid phases. For manufacturing the inventive compositions in tablet form, which can be monophasic or multiphasic, single colored or multicolored and/or consisting of one or several layers, all the ingredients—optionally for each layer—are preferably mixed together in a mixer and the mixture is compressed using conventional tablet presses, e.g. exocentric presses or rotating presses with compression forces in the range of about 50 to 100 kN/cm², preferably 60 to 70 kN/cm². Particularly for the case of multilayer tablets, it can be advantageous to precompress at least one layer. This is preferably carried out using compression forces between 5 and 20 kN/cm², particularly 10 to 15 kN/cm². Tablets prepared in this way preferably have a weight of 10 g to 50 g, particularly 15 g to 40 g. The tablets may be any shape—round, oval or angled—intermediate shapes also being possible.

It is particularly advantageous for multiphase compositions, that at least one of the phases comprises an amylase-sensitive material, especially starch, or is at least partially encapsulated or coated with this. In this way this phase is mechanically stabilized and/or protected against external influences and simultaneously attacked by an active amylase present in the wash liquor, such that the release of the ingredients is facilitated.

Similarly, preferred compositions according to the invention are characterized in that they are all in liquid, gel or paste form. The proteins, preferably a protein according to the invention, are added to such compositions and preferably result from a state of the art protein extraction and preparation in concentrated aqueous or non-aqueous solution, for example in liquid form, such as solution, suspension or emulsion, but also in gel form or encapsulated or as dried powder. This type of inventive washing or cleaning composition in the form of solutions in standard solvents is generally prepared by a simple mixing of the ingredients, which can be added in the substance or as a solution into an automatic mixer.

An embodiment of the present invention is such a liquid, gel or paste composition, to which has been added an encapsulated protein essential for the invention and/or one of the other comprised proteins and/or one of the other comprised ingredients in the form of microcapsules. Among these, those encapsulated with amylase-sensitive materials are particularly preferred. The use of a combination of amylase-sensitive materials and an amylolytic enzyme in a washing or cleaning agent can demonstrate synergistic effects in such a way that the starch degrading enzyme supports the breakdown of the microcapsule and thereby controls the release process of the encapsulated ingredients with the result that the release does not happen during storage and/or not at the beginning of the cleaning process, but rather at a defined time. By this mechanism, complex washing and cleaning agent systems can be based on the most varied ingredients and the most varied capsule types, which represent the particularly preferred embodiments of the present invention.

A comparable effect is given when the ingredients of the washing and cleaning composition are distributed in at least two different phases, for example two or more solid associated phases of a tableted washing or cleaning agent, or different granules in the same powdery agent. Two-phase or multi-phase cleaners are state of the art for use in both automatic dishwashers as well as washing agents. The activity of an amylolytic enzyme in an earlier activated phase is a prerequisite for the activation of a later phase, when this is surrounded by an amylase-sensitive shell or coating, or the amylase-sensitive material represents an integral part of the solid phase, whose partial or total hydrolysis disintegrates the relevant phase.

The ingredients of washing and cleaning compositions are able to suitably support each other's performance. Thus, it is known from the application WO 98/45396, that polymers, which can be added as cobuilders, such as, for example alkyl polyglycosides, can simultaneously stabilize and augment the activity and stability of included enzymes. Accordingly, it is preferred when the inventive bleaching system is modified by one of the customary ingredients mentioned above, especially stabilized and/or its contribution to the performance of the washing or cleaning composition is increased.

Processes for cleaning textiles or hard surfaces constitute a further subject of the invention and are characterized in that an above-described inventive bleaching system is active in at least one of the process steps.

In this embodiment, the invention is realized in that the inventively improved enzymatic properties are utilized in principal in terms of an improvement in each cleaning process. Each cleaning process is enhanced by the relevant activity when it is present in each process step. Such processes are realized in machines such as standard household automatic dishwashers or household washing machines. Further preferred processes are those wherein the inventive bleaching system is added in an above-described composition.

A further subject of the invention is a shampoo and/or a hair care agent comprising an inventive enzymatic bleaching system.

The shampoos and/or hair care products as well as bubble baths, shower baths, creams, gels, lotions, alcoholic and aqueous-alcoholic solutions, emulsions, wax/fatty masses, sticks, powder or salves, which include an inventive bleaching system, can contain mild surfactants, oils, emulsifiers, greases, pearlescent waxes, consistence providers, thickeners, polymers, silicone compounds, fats, waxes, stabilizers, biogenetic active principles, deodorants, antiperspirants, anti-dandruff agents, film formers, swelling agents, UV-light protection factors, antioxidants, hydrotropes, conserving agents, insect repellants, sun tans, solubilizers, perfume oils, colorants and the like as auxiliaries and additives.

Typical examples of suitable mild, i.e. particularly skin-compatible surfactants are fatty alcohol polyglycol ether sulfonates, monoglyceride sulfates, mono and/or dialkylsulfosuccinates, fatty acid isethionates, fatty acid sarcosinates, fatty acid taurides, fatty acid glutamates, α-olefin sulfonates, ether carboxylic acids, alkyl oligoglucosides, fatty acid glucamides, alkylamidobetaines and/or protein-fatty acid condensates, the last preferably on the basis of wheat proteins.

The following can be considered as oils, for example: Guerbet alcohols based on fatty alcohols with 6 to 18, preferably 8 to 10 carbon atoms, esters of linear C₆-C₂₂-fatty acids with linear C₆-C₂₂-fatty alcohols, esters of branched C₆-C₁₃-carboxylic acids with linear C₆-C₂₂-fatty alcohols, such as for example myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. In addition, suitable esters are esters of linear C₆-C₂₂-fatty acids with branched alcohols, especially 2-ethylhexanol, esters of hydroxycarboxylic acids with linear or branched C₆-C₂₂-fatty alcohols, especially dioctyl malate, esters of linear and/or branched fatty acids with polyhydroxy alcohols (e.g. propylene glycol, dimerdiol or trimertriol) and/or Guerbet alcohols, triglycerides based on C₆-C₁₀-fatty acids, liquid mono-/di-/triglyceride mixtures based on C₆-C₁₈-fatty acids, esters of C₆-C₁₈-fatty alcohols and/or Guerbet alcohols with aromatic carboxylic acids, especially benzoic acid, esters of C₂-C₁₂-dicarboxylic acids with linear or branched alcohols with 1 to 22 carbon atoms or polyols with 2 to 10 carbon atoms and 2 to 6 hydroxyl groups, vegetal oils, branched primary alcohols, substituted cyclohexanes, linear and branched C₆-C₂₂-fatty alcohol carbonates, Guerbet carbonates, esters of benzoic acid with linear and/or branched C₆-C₂₂-alcohols (e.g. Finsolv® TN), linear or branched, symmetrical or unsymmetrical dialkyl ethers with 6 to 22 carbon atoms per alkyl group, ring opening products of epoxidized fatty acid esters with polyols, silicone oils and/or aliphatic or naphthenic hydrocarbons, such as, for example squalane, squalene or dialkylcyclohexanes.

Emulsifiers can be selected for example from nonionic surfactants from at least one of the following groups:
(1) Addition products of 2 to 30 moles ethylene oxide and/or 0 to 5 moles propylene oxide to fatty alcohols with 8 to 22 carbon atoms, to fatty acids with 12 to 22 carbon atoms, to alkyl phenols with 8 to 15 carbon atoms in the alkyl group as well as alkylamines with 8 to 22 carbon atoms in the alkyl radical;
(2) C_{12/18}-fatty acid mono and diesters of addition products of 1 to 30 moles ethylene oxide on glycerin;
(3) Glycerin mono and diesters and sorbitol mono and diesters of saturated and unsaturated fatty acids with 6 to 22 carbon atoms and their ethylene oxide addition products;
(4) Alkyl- and/or alkenyl mono and -oligoglycosides with 8 to 22 carbon atoms in the alk(en)yl radical and their ethoxylated analogs;
(5) Addition products of 15 to 60 moles ethylene oxide on castor oil and/or hydrogenated castor oil;
(6) Polyol esters and especially polyglycerine esters;
(7) Addition products of 2 to 15 moles ethylene oxide on castor oil and/or hydrogenated castor oil;
(8) Partial esters based on linear, branched, unsaturated or saturated C_{6/22}-fatty acids, ricinoleic acid as well as 12-hydroxystearic acid and glycerin, polyglycerin, pentaerythritol, dipentaerythritol, sugar alcohols (e.g. sorbitol), alkyl glucosides (e.g. methyl glucoside, butyl glucoside, lauryl glucoside) as well as polyglucosides (e.g. cellulose);
(9) Mono, di and trialkyl phosphates as well as mono, di and/or tri-PEG-alkylphosphates and salts thereof;
(10) Wool wax alcohols;
(11) Polysiloxane-polyalkyl-polyether copolymers or corresponding derivatives;
(12) Mixed esters of pentaerythritol, fatty acids, citric acid and fatty alcohol according to the Patent DE 1165574 and/or mixed esters of fatty acids with 6 to 22 carbon atoms, methyl glucose and polyols, preferably glycerine or polyglycerine,
(13) Polyalkylene glycols and
(14) Glycerine carbonate.

The addition products of ethylene oxide and/or propylene oxide on fatty alcohols, fatty acids, alkyl phenols, glycerin mono and diesters as well as sorbitol mono and diesters of fatty acids or on castor oil represent known, commercially available products. They can be considered as mixtures of homologs, whose mean degree of alkoxylation corresponds to the ratio of amounts of ethylene oxide and/or propylene oxide, used for the addition reaction, and that of the substrate. C_{12/18} fatty acid mono and diesters of addition products of ethylene oxide on glycerine are known from the patent DE 2024051 as greasing agents for cosmetic preparations.

Alkyl and/or alkenyl mono and oligoglycosides, their manufacture and use are known from the prior art. Their manufacture results particularly from the reaction of glucose or oligosaccharides with primary alcohols containing 8 to 18 carbon atoms. As far as the glycoside groups are concerned, both monoglycosides, in which a cyclic sugar group is glycosidically linked to the fatty alcohol, and also oligomeric glycosides, with a degree of oligomerization of preferably about 8, are suitable. In this context, the oligomerization degree is a statistical mean value based on the typical homolog distribution of such technical products.

Typical examples of suitable polyglycerine esters are polyglyceryl-2 dipolyhydroxystearate (Dehymuls® PG PH), polyglycerin-3-diisostearate (Lameform® TGI), polyglyceryl-4-isostearate (Isolan® GI 34), polyglyceryl-3 oleate, diisostearoyl polyglyceryl-3 diisostearate (Isolan® PDI), polyglyceryl-3 methylglucose distearate (Tego Care® 450), polyglyceryl-3 beeswax (Cera Bellina®), polyglyceryl-4 caprate (polyglycerol caprate T2010/90), polyglyceryl-3 cetyl ether (Chimexane® (NL), polyglyceryl-3 distearate (Cremophor® GS 32) and polyglyceryl polyricinoleate (Admul® WOL 1403) polyglyceryl dimerate isostearate and mixtures thereof.

Moreover, zwitterionic surfactants can be used as emulsifiers. Zwitterionic surfactants are designated as those surface-active compounds that carry at least a quaternary ammonium group and at least a carboxylate and a sulfonate group in the molecule. Particularly suitable zwitterionic surfactants are the so-called betaines such as the N-alkyl-N,N-dimethyl ammonium glycinates, for example the cocoalkyl dimethyl ammonium glycinate, N-acylaminopropyl-N,N-dimethyl ammonium glycinates, for example the cocoacylaminopropyl dimethyl ammonium glycinate, and 2-alkyl-3-carboxymethyl-3-hydroxyethyl imidazolines with 8 to 18 carbon atoms in each of the alkyl or acyl groups, as well as cocoacylaminoethylhydroxyethylcarboxymethyl glycinate. The known fatty acid derivative known under the CTFA-description Cocamidopropyl Betaine is particularly preferred. The ampholytic surfactants are understood to include such surface-active compounds that apart from a C_{8/18} alkyl or acyl group, contain at least one free amino group and at least one COOH or SO₃H group in the molecule, and are able to form internal salts. Examples of suitable ampholytic surfactants are N-alkylglycines, N-alkylpropionic acids, N-alkylaminobutyric acids, N-alkyliminodipropionic acids, N-hydroxyethyl-N-alkylamidopropylglycine, N-alkyltaurines, N-alkylsarcosinea, 2-alkyl-aminopropionic acids und alkylaminoacetic acids with about 8 to 18 C-atoms in each alkyl group. Particularly preferred ampholytic surfactants are N-cocoalkylamino propionate, cocoacylaminoethylamino propionate and C_{12/18}-acyl sarcosine. Beside the ampholytics, the quaternary emulsifiers can also be considered, wherein the esterquats, preferably methyl quaternized difatty acid triethanolamine ester salts are particularly preferred.

As greasing agents, substances such as lanolin and lecithin, as well as polyethoxylated or acylated lanolin and lecithin derivatives, polyol fatty acid esters, monoglycerides and fatty acid alkanolamides can be used, the last ones serving as foam stabilizers at the same time.

Pearlescent waxes include: alkylene glycol esters, especially ethylene glycol distearate; fatty acid alkanolamides, especially cocofatty acid diethanolamide; partial glycerides, especially monoglycerides of stearic acid; esters of polyfunctional, optionally hydroxy-substituted carboxylic acids with fatty alcohols with 6 to 22 carbon atoms, especially long chain esters of tartaric acid; solids, such as, for example fatty alcohols, fatty ketones, fatty aldehydes, fatty ethers and fatty carbonates, which have a total of at least 24 carbon atoms, especially lauron and distearyl ether; fatty acids like stearic acid, hydroxystearic acid or behenic acid, ring opened products of olefin epoxides having 12 to 22 carbon atoms with fatty alcohols with 12 to 22 carbon atoms and/or polyols having 2 to 15 carbon atoms and 2 to 10 hydroxyl groups and mixtures thereof.

Consistence agents primarily include fatty alcohols or hydroxyfatty alcohols having 12 to 22 and preferably 16 to 18 carbon atoms, besides partial glycerides, fatty acids or hydroxyfatty acids. A combination of these materials with alkyl oligoglucosides and/or fatty acid N-methylglucamides of the same chain length and/or polyglycerine poly-12-hydroxystearates is preferred.

Suitable thickeners are for example aerosil types (hydrophilic silicic acids), polysaccharides, especially xanthane gum, guar-guar, agar-agar, alginates and tyloses, carboxymethyl cellulose and hydroxyethyl cellulose, in addition, higher molecular polyethylene glycol mono- and-diesters of fatty acids, polyacrylates, (e.g. Carbopole® from Goodrich or Synthalene® from Sigma), polyacrylamides, Polyvinyl alcohol and polyvinyl pyrrolidone, surfactants such as ethoxylated fatty acid glycerides, esters of fatty acids with polyols such as pentaerythritol or trimethylolpropane, fatty alcohol lethoxylates with narrowed homolog distribution or alkyl oligoglucosides as well as electrolytes like cooking salt and ammonium chloride.

Exemplary suitable cationic polymers are cationic cellulose derivatives, such as a quaternized hydroxyethyl cellulose, available under the trade name Polymer JR 400® from Amerchol, cationic starches, copolymers of diallylammonium salts and acrylamides, quaternized vinyl pyrrolidone/vinyl imidazole polymers, like e.g. Luviquat® (BASF), condensation products of polyglycols with amines, quaternized collagen polypeptides, like for example, lauryldimonium hydroxypropyl hydrolyzed collagen (Lamequat®L/Grunau), quaternized wheat polypeptides, polyethylene imines, cationic silicone polymers, such as amidomethicone, copolymers of adipic acid and dimethylaminohydroxypropyldiethylene triamine (Cartaretine®/Sandoz), copolymers of acrylic acid and dimethyl diallyl ammonium chloride (Merquat® 550/Chemviron), polyaminopolyamides, such as e.g. described in FR 2252840 A as well as their crosslinked water-soluble polymers, cationic chitin derivatives such as e.g. quaternized chitosan, optionally microcystallinically dispersed, condensation products of dihaloalkylenes, such as e.g. dibromobutane with bisdialkylamines, such as e.g. bis-dimethylamino-1,3-propane, cationic guar gum, such as e.g. Jaguar® CBS, Jaguar® C-17, Jaguar® C-16 from Celanese company, quaternized ammonium polymers, such as e.g. Mirapol® A-15, Mirapol® AD-1, Mirapol® AZ-1 from the Miranol company.

Anionic, zwitterionic, amphoteric and nonionic polymers include, for example, vinyl acetate-crotonic acid copolymers, vinyl pyrrolidone-vinyl acrylate copolymers, vinyl acetate-butyl maleate-isobornyl acrylate copolymers, methyl vinyl ether-maleic anhydride copolymers and their esters, uncrosslinked polyacrylic acids and those crosslinked with polyols, acrylamidopropyl trimethyl ammonium chloride-acrylate copolymers, octylacylamide-methyl methacrylate-tert.-butylaminoethyl methacrylate-2-hydroxypropyl methacrylate copolymers, polyvinyl pyrrolidone, vinyl pyrrolidone-vinyl acetate copolymers, vinyl pyrrolidone-dimethylaminoethyl methacrylate-vinyl caprolactam terpolymers as well as optionally derivatized cellulose ethers and silicones.

Exemplary suitable silicone compounds are dimethylpolysiloxanes, methylphenylpolysiloxanes, cyclic siloxanes as well as amino-, fatty acid-, alcohol-, polyether-, epoxy-, fluorine-, glycoside- and/or alkyl modified silicone compounds, which may be both liquid or also resinous at room temperature. Simethicones, which are mixtures of dimethicones having an average chain length of 200 to 300 dimethylsiloxane units and hydrated silicates, are also suitable. A detailed review of suitable volatile silicones is found in Todd et al., Cosm. Toil. 91, 27 (1976).

Typical examples of fats are glycerides; waxes include inter alia natural waxes such as e.g. candelilla wax, carnauba wax, japan wax, esparto grass wax, cork wax, guarum wax, rice oilseed wax, raw sugar wax, ouricury wax, montan wax, beeswax, shellac wax, spermaceti, lanolin (wool wax), fowl fat, ceresine, ozokerite, petrolatum, paraffin waxes microwaxes; chemically modified waxes (hard waxes), such as e.g. montan ester waxes, sasol waxes, hydrogenated jojoba waxes as well as synthetic waxes, such as e.g. polyalkylene waxes and polyethylene glycol waxes.

Metal salts of fatty acids, such as e.g. magnesium-, aluminum- and/or zinc stearate or ricinoleate can be used as stabilizers.

Biogenetic active agents are understood to mean for example, tocopherol, tocopherol acetate, tocopherol palmitate, ascorbic acid, desoxyribonucleic acid, retinol, bisabolol, allantoin, phytanetriol, panthenol, AHA-acids, amino acids, ceramides, pseudoceramides, essential oils, plant extracts and vitamin complexes.

Cosmetic deodorants act against body odors, masking or eliminating them. Body odors result from the action of skin bacteria on apocrine sweat, whereby unpleasant smelling degradation products are formed. Accordingly, deodorants contain active principles, which act as germicides, enzyme inhibitors, odor absorbers or odor masks.

As germicides, which can be optionally added to the cosmetics according to the invention, basically all substances that are active against gram-positive bacteria are suitable, such as e.g. 4-hydroxybenzoic acid and its salts and esters, N-(4-chlorophenyl)-N′-(3,4-dichlorophenyl)urea, 2,4,4′-trichloro-2′-hydroxydiphenyl ether (Triclosan), 4-chloro-3,5-dimethylphenol, 2,2′-methylene-bis(6-bromo-4-chlorophenol), 3-methyl-4-(1-methylethyl)phenol, 2-benzyl-4-chlorophenol, 3-(4-chlorophenoxy)-1,2-propanediol, 3-iodo-2-propinylbutyl carbamate, chlorhexidine, 3,4,4′-trichlorocarbanilide (TTC), antibacterial fragrances, menthol, mint oil, phenoxyethanol, glycerin monolaurate (GML), diglycerin monocaprinate (DMC), salicylic acid-N-alkylamides such as, e.g. salicylic acid n-octylamide or salicylic acid n-decylamide.

Enzyme inhibitors can also be added to the inventive cosmetics. Examples of possible suitable enzyme inhibitors are esterase inhibitors. Trialkyl citrates are preferred, such as trimethyl citrate, tripropyl citrate, triisopropyl citrate, tributyl citrate and particularly triethyl citrate (Hydagen® CAT, Henkel KgaA, Düsseldorf/Germany). The substances inhibit the enzymatic activity and thereby reduce the odor formation. Additional substances that can be considered as esterase inhibitors are sterol sulfates or phosphates, such as e.g. lanosterin-, cholesterin-, campesterin-, stigmasterin- and sitosterin sulfate or phosphate, dicarboxylic acids and their esters, such as e.g. glutaric acid, monoethyl glutarate, diethyl glutarate, adipic acid, monoethyl adipate, diethyl adipate, malonic acid and diethyl malonate, hydroxycarboxylic acids and their esters such as e.g. citric acid, malic acid, tartaric acid or diethyl tartrate, as well as zinc glycinate.

Suitable odor absorbers are substances, which take up the odor forming compounds and firmly block them. They reduce the partial pressures of the individual components and thus also reduce their rate of propagation. It is important that the perfumes remain unaffected by this. Odor absorbers have no activity against bacteria. The comprise as the major component, for example, a complex zinc salt of ricinoleic acid or special, largely odor-neutral fragrances, which are known to the expert as fixing agents, such as e.g. extracts of labdanum or styrax or specific abietic acid derivatives. Fragrances or perfume oils act as masking agents and in addition to their function as masking agents, lend the deodorants their particular fragrance. Exemplary perfume oils include mixtures of natural and synthetic aromas. Natural aromas are extracts of flowers, stalks and leaves, fruits, fruit skins, roots, branches, herbs and grasses, needles and twigs as well as resins and balsams. In addition, animal materials such as e.g. civet and castoreum can be considered. Typical synthetic aroma compounds are products of the type of the esters, ethers, aldehydes, ketones, alcohols and hydrocarbons.

Antiperspirants reduce sweat formation by influencing the activity of the ecrinal sweat glands and thereby act against armpit moisture and body odor. Aqueous or anhydrous formulations of antiperspirants typically contain the following ingredients:
(a) astringent principles,
(b) oil components
(c) nonionic emulsifiers,
(d) co emulsifiers,
(e) structurants,
(f) auxiliaries such as e.g. thickeners or complexing agents and/or
(g) non-aqueous solvents such as e.g. ethanol, propylene glycol and/or glycerine.

Salts of aluminum, zirconium or zinc are the main suitable astringent antiperspirant active principles. Such suitable antihydrotically active substances are e.g. aluminum chloride, hydrated aluminum chloride, hydrated aluminum dichloride, hydrated aluminum sesquichloride and their complexes e.g. with 1,2-propylene glycol, aluminum hydroxy allantoinate, aluminum chloride tartrate, aluminum-zirconium trichlorohydrate, aluminum-zirconium tetrachlorohydrate, aluminum-zirconium pentachlorohydrate and their complexes e.g. with amino acids such as glycine.

The antiperspirants can also comprise standard oil-soluble and water-soluble auxiliaries in minor amounts. Such oil-soluble auxiliaries can be for example:
anti-inflammatory, skin protecting or fragrant essential oils,
synthetic, skin-protecting active substances and/or
oil-soluble perfume oils.

Typical water-soluble additives are e.g. conservers, water-soluble aromas, pH adjustors, e.g. buffer mixtures, water-soluble thickeners, e.g. water-soluble natural or synthetic polymers such as e.g. xanthane gum, hydroxyethyl cellulose, polyvinyl pyrrolidone or high-molecular polyethylene oxides.

Climbazole, octopirox and zinc pyrethion can be used as anti-dandruff agents.

Usable film builders are for example, chitosan, microcrystalline chitosan, quaternized chitosan, polyvinyl pyrrolidone, vinyl pyrrolidone-vinyl acetate copolymers, polymers of the acrylic acid series, quaternized cellulose derivatives, collagen, hyaluronic acid or its salts and similar compounds.

As swelling agents for the aqueous phase, montmorillonite, mineral clays, Pemulen® as well as Carbopol types (Goodrich) can be used. Additional suitable polymers or swelling agents can be found in the review by R. Lochhead in Cosm. Toil. 108, 95 (1993).

The UV light protective factors are understood for example to be organic substances (protective light filters) that are liquid or solid at room temperature and which are able to absorb UV radiation and emit the resulting energy in the form of longer wavelength radiation, for example as heat. UVB filters can be oil-soluble or water-soluble. As oil-soluble substances, the following may be cited:
3-benzylidenecamphor or 3-benzylidenenorcamphor and its derivatives, for example 3-(4-methylbenzylidene)camphor, as described in the EP 0693471 B1;
4-aminobenzoic acid derivatives, preferably 4-(dimethylamino)benzoic acid, 2-ethylhexyl ester, 4-(dimethylamino)benzoic acid, 2-octyl ester and 4-(dimethylamino)benzoic acid, amyl ester;
esters of cinnamic acid, preferably 4-methoxycinnamic acid, 2-ethylhexyl ester, 4-methoxycinnamic acid, propyl ester, 4-methoxycinnamic acid, isoamyl ester, 2-cyano-3,3-phenylcinnamic acid, 2-ethylhexyl ester (octocrylene);
esters of salicylic acid, preferably salicylic acid, 2-ethylhexyl ester, salicylic acid, 4-isopropylbenzyl ester, salicylic acid, homomenthyl ester;
derivatives of benzophenone, preferably 2-hydroxy-4-methoxybenzophenone, 2-hydroxy-4-methoxy-4′-methylbenzophenone, 2,2′-dihydroxy-4-methoxybenzophenone;
esters of benzalmalonic acid, preferably 4-methoxybenzmalonic acid, di-2-ethylhexylester;
triazine derivatives, such as, for example 2,4,6-trianilino-(p-carbo-2′-ethyl-1′-hexyloxy)-1,3,5-triazine and octyl triazone, as described in EP 0818450 A1 or dioctyl butamidotriazone (Uvasorb® HEB);
propane-1,3-dione, such as for example 1-(4-tert.-butylphenyl)-3-(4′-methoxyphenyl)propane-1,3-dione;
ketotricyclo(5.2.1.0)decane derivatives, as described in EP 0694521 B1.

Water-soluble substances include:
2-phenylbenzimidazole-5-sulfonic acid and its alkali-, earth alkali-, ammonium-, alkyl ammonium-, alkanol ammonium- and glucammonium salts;
sulfonic acid derivatives of benzophenones, preferably 2-hydroxy-4-methoxybenzophenone-5-sulfonic acid and its salts;
sulfonic acid derivatives of 3-benzylidenecamphor, as for example 4-(2-oxo-3-bornylidenemethyl)benzene sulfonic acid and 2-methyl-5-(2-oxo-3-bornylidene)sulfonic acid and its salts.

Typical UV-A filters particularly include derivatives of benzoylmethane, such as, for example 1-(4′-tert.-butylphenyl)-3-(4′-methoxyphenyl)propane-1,3-dione, 4-tert.-butyl-4′-methoxydibenzoylmethane (Parsol 1789), 1-phenyl-3-(4′-isopropylphenyl)-propane-1,3-dione as well as enamine compounds, as described in the DE 19712033 A1 (BASF). Naturally, the UV-A and UV-B filters can also be added as mixtures. Beside the cited soluble materials, also insoluble, light protective pigments, namely finely dispersed, preferably, nano metal oxides or salts can be considered for this task. Exemplary suitable metal oxides are particularly zinc oxide and titanium oxide and also oxides of iron, zirconium, silicon, manganese, aluminum and cerium as well as their mixtures. Silicates (talc), barium sulfate or zinc stearate cab be added as salts. The oxides and salts are already used in the form of pigments for skin care and skin protecting emulsions and decorative cosmetics. Here, the particles should have an average diameter of less than 100 nm, preferably between 5 and 50 nm and especially between 15 and 30 nm. They can be spherical, however elliptical or other shaped particles can be used. The pigments can also be surface treated, i.e. hydrophilized or hydrophobized. Typical examples are coated titanium dioxides, such as, for example Titandioxid Z 805 (Degussa) or Eusolex® T2000 (Merck); hydrophobic coating agents preferably include trialkoxy octylsilanes or Simethicones. Sun-protection agents preferably contain micropigments or nano-pigments. Micronized zinc oxide is preferably used. Further suitable UV light protection filters may be found in the review by P. Finkel in SöFW-Journal, Volume 122 (1996), p. 543.

As well as both above-cited groups of primary light protective materials, secondary light protective agents of the antioxidant type can also be used, which interrupt photochemical chain reactions that are propagated when the UV-radiation penetrates the skin. Typical examples are amino acids (e.g. glycine, histidine, tyrosine, tryptophan) and their derivatives, imidazoles (e.g. urocanic acid) and their derivatives, peptides such as D,L-carnosine, D-carnosine, L-carnosine and their derivatives (e.g. anserine), carotinoides, carotines (e.g. α-carotine, β-carotine, lycopine) and their derivatives, chlorogenic acid and their derivatives, liponic acid and their derivatives (e.g. dihydroliponic acid), aurothioglucose, propylthiouracil and other thioles (e.g. thioredoxine, glutathione, cystein, cystine, cystamine and their glycosyl-, n-acetyl-, methyl-, ethyl-, propyl-, amyl-, butyl- and lauryl-, palmitoyl-, oleyl-, γ-linoleyl-, cholesteryl- and glyceryl esters) as well as their salts, dilauryl thiodipropionate, distearyl thiodipropionate, thiodipropionic acid and their derivatives (esters, ethers, peptides, lipids, nucleotides, nucleosides and salts) as well as sulfoximine compounds (e.g. buthioninesulfoximines, homocysteinsulfoximine, butionine sulfone, penta-, hexa-, heptathionine sulfoximine) in very minor compatible doses (e.g. pmol to μmol/kg), further (metal)-chelates (e.g. α-hydroxyfatty acids, palmitic acid, phytinic acid, lactoferrin), α-hydroxyacids (e.g. citric acid, lactic acid, malic acid), humic acid, gallic acid, gall extracts, bilirubin, biliverdin, EDTA, EGTA and their derivatives, unsaturated fatty acids and their derivatives (e.g. γ-linolenic acid, linolic acid, oleic acid), folic acid and their derivatives, ubiquinone and ubiquinol and their derivatives, vitamin C and derivatives (e.g. ascorbyl palmitate, Mg-ascorbyl phosphate, ascorbyl acetate), tocopheroles and derivatives (e.g. vitamin-E-acetate), vitamin A and derivatives (vitamin-A-palmitate) as well as coniferyl benzoate of benzoic resin, rutinic acid and their derivatives, α-glycosylrutine, ferula acid, furfurylideneglucitol, carnosine, butylhydroxytoluene, butylhydroxyanisol, nordihydroguajac resin acid, nordihydroguajaret acid, trihydroxybutyrophenone, uric acid and their derivatives, mannoses and their derivatives, superoxide-dismutase, zinc und its derivatives (e.g. ZnO, ZnSO4) selenium and its derivatives (e.g. selenium-methionine), stilbenes and their derivatives (e.g. stilbene oxide, trans-stilbene oxide) and the inventive suitable derivatives (salts, esters, ethers, sugars, nucleotides, nucleosides, peptides and lipids) of these cited active substances.

To improve the flow properties, hydrotropes can also be added, such as, for example, ethanol, isopropyl alcohol, or polyols. Polyols, which are considered, possess preferably 2 to 15 carbon atoms and at least two hydroxyl groups. The polyols can comprise further functional groups, especially amino groups, or can be modified by nitrogen. Typical examples are
glycerine;
alkylene glycols, such as, for example ethylene glycol, diethylene glycol, propylene glycol, butylene glycol, hexylene glycol as well as polyethylene glycols with an average molecular weight of 100 to 1000 daltons;
technical oligoglycerine mixtures with a self condensation degree of 1.5 to 10 about like technical diglycerine mixtures with a diglycerine content of 40 to 50wt. %;
methylol compounds, particularly like trimethylolethane, trimethylolpropane, trimethylolbutane, pentaerythritol and dipentaerythritol;
lower alkyl glucosides, particularly those with 1 bis 8 carbons in the alkyl group, such as, for example methyl- and butyl glucoside;
sugar alcohols having 5 to 12 carbon atoms, such as, for example sorbitol or mannitol,
sugars with 5 to 12 carbon atoms, such as, for example glucose or saccharose;
aminosugars, such as, for example glucamine;
dialcoholamines, like diethanolamine or 2-amino-1,3-propanediol.

Suitable preservatives are, for example phenoxyethanol, formaldehyde solution, parabene, pentanediol or sorbic acid as well as the further classes of materials described in Appendix 6, part A and B of the Cosmetic Regulation. Insect repellants include N,N-diethyl-m-toluamide, 1,2-pentanediol or ethyl butylacetylaminopropionate; suitable self tanning agents include dihydroxyacetone.

As perfume oils, the known mixtures of natural and synthetic aromas can be cited. Natural aromas are extracts of flowers (lilies, lavender, roses, jasmine, neroli, ylang ylang), stalks and leaves (geranium, patchouli, petit grain), fruits (aniseed, coriander, caraway, juniper), fruit skins (bergamot, lemons, oranges), roots (mace, angelica, celery, cardamom, costic, iris, calmus), wood (pine, sandal, guava, cedar, rose wood), herbs and grasses (tarragon, lemon grass, sage, thyme), needles and twigs (spruce, fir, scotch pine, larch), resins and balsam (galbanum, elemi, benzoin, myrrh, olibanum, opoponax). In addition, animal raw materials can be considered, such as civet and castoreum. Typical synthetic aroma compounds are products of the ester, ether, aldehyde, ketone, alcohol and hydrocarbon types.

As colorants, those substances suitable and approved for cosmetic purposes can be used, as summarized, for example in the publication “Kosmetische Färbemittel” of the Colorant Commission of the Deutsche Forschungsgemeinschaft, Verlag Chemie, Weinheim, 1984, p. 81-106. These colorants are typically used in concentrations of 0.001 to 0.1 wt. %, based on the total mixture.

The total content of auxiliaries and additives can be 1 to 50, preferably 5 to 40 wt. %, based on the composition. The manufacture of the composition can be made using customary cold or hot processes; preferably according to the phase inversion temperature method.

A further subject of the invention is an oxidative colorant for dyeing keratin fibers, comprising an inventive enzymatic bleaching system. Keratin fibers are understood to mean wool, feathers, skins and particularly human hair.

For the manufacture of the inventive oxidizing agents, the oxidative colorant precursors, together with the choline oxidases are mixed together in a suitable aqueous carrier under the exclusion of oxygen from the air. Such carriers are e.g. thickened aqueous solutions, creams (emulsions), gels or surfactant-containing foam preparations, e.g. shampoos or foam aerosols or other preparations that are suitable for application on hair.

Basically, anhydrous powders are also suitable as carriers; in this case, the oxidative colorants are dispersed or dissolved in water immediately before use. Preferred components of carriers that are used are
wetting agents and emulsifiers
thickeners
reducing agents (antioxidants)
hair care additives
fragrances and
solvents such as e.g. water, glycols or lower alcohols.

Suitable wetting agents and emulsifiers are e.g. anionic, zwitterionic, ampholytic and nonionic surfactants. Cationic surfactants can also be used to obtain specific effects.

Suitable thickeners are the water-soluble high-molecular polysaccharide derivatives or polypeptides, e.g. cellulose ethers or starch ethers, gelatin, plant gums, biopolymers (xanthane gum) or water-soluble synthetic polymers such as e.g. polyvinyl pyrrolidone, polyvinyl alcohol, polyethylene oxides, polyacrylamides, polyurethanes, polyacrylates and others.

In addition, surfactant-containing preparations can also be thickened by solubilization or emulsification of polar lipids. Such lipids are e.g. fatty alcohols with 12-18 carbon atoms, (free) fatty acids with 12-18 carbon atoms, partial glycerides of fatty acids, sorbitol esters of fatty acids, fatty acid alkanolamides, lower oxyethylated fatty acids or fatty alcohols, lecithin, sterin. Finally, carriers in gel form can also be produced on the basis of aqueous soap gels e.g. ammonium oleate.

Reducing agents (antioxidants), which are added to the carrier, in order to prevent a premature oxidative development of the colorant before its use on the hair, are e.g. sodium sulfate or sodium ascorbate.

Hair care ingredients can include, for example, fats, oils or waxes in emulsified form, structuring additives, such as, for example, glucose or pyridoxine, brightening components, for example, water-soluble proteins, protein degradation products, amino acids, water-soluble cationic polymers, silicones, vitamins, panthenol or plant extracts.

Finally, fragrances and solvents, for example, glycols such as 1,2-propylene glycol, glycerol, gycol ethers such as butyl glycol, ethyl diglycol or lower monohydroxy alcohols such as ethanol or isopropanol, can also be ingredients.

In addition, other additives can also be present in order to improve the stability and usage properties of the oxidizing dyes, for example, complexing agents such as EDTA, NTA or organophosphonates, swelling and penetrating agents, such as, for example urea, guanidine, bicarbonates, buffer salts such as, for example, ammonium chloride, ammonium nitrate, ammonium sulfate or alkyl ammonium salts and propellants if required.

A further subject of the invention is as an agent for the care of the mouth, teeth or dentures, especially denture cleaners, containing inventively employable bleaching systems for bleaching or for disinfection.

In the case of partial prostheses or dentures, presentation as denture cleaning tablets or as mouthwash or rinse or toothpaste is also suitable.

The mouth, tooth or denture care agents according to the invention can be available for example as a mouthwash, gel, liquid tooth cleaner, firm toothpaste, denture cleaner or denture adhesive cream.

For this, the inventively useable bleaching systems need to be incorporated within a suitable carrier.

For example, preparations in powder form or water-alcohol solutions can serve as carriers, which can contain 0 to 15 weight % ethanol, 1 to 1.5 weight % flavor oils and 0.01 to 0.5 weight % sweeteners for mouthwash or 15 to 60 weight % ethanol, 0.05 to 5 weight % flavor oils, 0.1 to 3 weight % sweeteners as well as other additives if required for mouthwash concentrate which is diluted with water before use. The concentration of the components must be selected at a high enough level, such that following dilution the concentration during use does not fall below the lower limit of the range mentioned.

Gels as well as more or less flowable pastes, which are squeezed out of flexible plastic containers or tubes and applied to the teeth with the aid of a toothbrush, can also serve as carriers. Such products contain higher quantities of wetting and binding agents or stabilizers and polishing agents. Furthermore, perfume oils, sweeteners and water also contained in these preparations.

Moisturizers can include, for example, glycerol, sorbitol, xylite, propylene glycols, polyethylene glycols or mixtures of these polyols, especially polyethylene glycols with a molecular weight from 200 to 800 (from 400-2000). The preferred wetting agent is sorbitol in a quantity of 25-40 weight %.

Condensed phosphates in the form of their alkali salts, preferably in the form of the sodium or potassium salt, can be included as anti-tartar agents and as inhibitors of demineralization. Aqueous solutions of these phosphates react as alkalis due to hydrolytic effects. On addition of acid, the pH of the mouth, tooth and/or denture care agents according to the invention is kept within the preferred range of 7.5-9. A mixture of various condensed phosphates or also hydrated salts of condensed phosphates can be used. However, the specified quantities of 2-12 weight % refer to the anhydrous salts. The preferred condensed phosphate is a sodium or potassium tripolyphosphate in a quantity of 5-10% by weight of the composition.

A preferred active ingredient is a caries-inhibiting fluorine compound, preferably from the fluoride or monofluorophosphate group in a quantity of 0.1-0.5 weight % fluorine. Suitable fluorine compounds include, for example, sodium monofluorophosphate (Na₂PO₃F), potassium monofluorophosphate, sodium or potassium fluoride, stannous fluoride or a fluoride of an organic amino compound.

Binders and consistency regulators include, for example, natural and synthetic water-soluble polymers such as carrageenan, traganth, guar, starch and their non-ionic derivatives such as hydroxypropyl guar, hydroxyethyl starch, cellulose ethers such as hydroxyethyl cellulose or methylhydroxypropyl cellulose. Agar-agar, xanthane gum, pectins, water-soluble carboxyvinyl polymers (e.g. Carbopol® types), polyvinyl alcohol, polyvinyl pyrrolidone, high-molecular-weight polyethylene glycols (molecular weight 10³ to 10⁶ D) can also be used. Other materials suitable for controlling viscosity include layered silicates, for example montmorillonite, colloidal swelling silicas, e.g. aerogel silicas or pyrogenic silicas.

Polishing components can include all polishing agents that are known for this purpose, but preferably precipitated and gel silicas, aluminum hydroxide, aluminum silicate, aluminum oxide, aluminum oxide trihydrate, insoluble sodium metaphosphate, calcium pyrophosphate, dicalcium phosphate, chalk, hydroxyapatite, hydrotalcite, talcum, magnesium aluminum silicate (Veegum®), calcium sulfate, magnesium carbonate, magnesium oxide, sodium aluminum silicates, for example zeolite A or organic polymers, for example polymethacrylate. The polishing agents are preferably used in smaller quantities, for example 1-10 weight %.

The tooth and/or mouth care products according to the invention can have their organoleptic qualities improved by the addition of flavor oils and sweeteners. Flavor oils can include all natural and synthetic flavorings used in products for the care of mouth, teeth or dentures. Natural flavors can be used in the form of ethereal oils isolated from the drugs or in the form of individual components isolated from the latter. Preferably, at least one flavor oil from the group of peppermint oil, curled mint oil, anise oil, caraway oil, eucalyptus oil, fennel oil, cinnamon oil, geranium oil, sage oil, thyme oil, marjoram oil, basil oil, citrus oil, wintergreen oil, or one or more synthetically produced components isolated from these oils should be included. The most important components of the abovementioned oils are, for example, menthol, carol, anethole, cineol, eugenol, cinnamaldehyde, geraniol, citronellol, linalool, salvene, thymol, terpinene, terpinol, methyl chavicol and methyl salicylate. Other suitable flavorings include, for example, menthyl acetate, vanillin, ionone, linalyl acetate, rhodinol and piperitone. Natural sugars such as sucrose, maltose, lactose and fructose or synthetic sweeteners such as sodium saccharin, sodium cyclamate or aspartame can be used for sweetening.

Surfactants that can be used include alkyl- and/or alkenyl-(oligo)-glycosides in particular. Their manufacture and use as surfactants is well known for example from U.S. Pat. No. 3,839,318, U.S. Pat. No. 3,707,535, U.S. Pat. No. 3,547,828 DE-A-19 43 689, DE-A-20 36 472 and DE-A-30 01 064 as well as from EP-A-77 167. With respect to glycoside residues, both monoglycosides (x =1), in which a pentose or hexose residue is glycosidically bound to a primary alcohol of 4-16 C atoms, and oligomeric glycosides with a degree of oligomerization of up to 10 are suitable. In this case the degree of oligomerization is a statistical average, which in such technical products is usually based on a homologous distribution.

Suitable alkyl and/or alkenyl-(oligo)-glycosides are preferably of the formula RO(C₆H₁₀O)ₓ—H, in which R is an alkyl-and/or alkenyl group with 8 to 14 C atoms and x has an average value of 1 to 4. Alkyl-oligo-glucosides based on hydrogenated C_{12/14} coconut alcohol with a DP of 1 to 3 are especially suitable. Alkyl- and/or alkenyl-glycoside surfactants can be used very economically, amounts of 0.005 up to 1 weight % being sufficient.

In addition to the abovementioned alkyl glucoside surfactants, other non-ionic, amphoteric and cationic surfactants can be included, such as: fatty alcohol polyglycol ether sulfates, monoglyceride sulfates, monoglyceride ether sulfates, mono- and/or dialkyl sulfosuccinates, fatty acid isothionates, fatty acid sarcosinates, fatty acid taurides, fatty acid glutamates, ether carboxylic acids, fatty acid glucamides, alkyl amido betaine and/or protein/fatty acid condensates, the latter preferably being based on wheat proteins. Especially when dissolving the mainly water-insoluble flavor oils, one may need to use a non-ionic solvent from the surfactant group. Oxyethylated fatty acid glycerides, oxyethylated fatty acid sorbitol partial esters or fatty acid partial esters of glycerol or sorbitol oxethylates are especially suitable for this purpose. Solvents from the oxethylated fatty acid glyceride group include, above all, all the addition products of 20 to 60 moles ethylene oxide with mono- and diglycerides of linear fatty acids having 12 to 18 C atoms or with triglycerides of hydroxy fatty acids such as oxystearic acid or ricinoleic acid. Other suitable solvents include oxyethylated fatty acid sorbitol partial esters; preferably adsorption products from 20 to 60 mol ethylene oxide with sorbitol monoesters and sorbitol diesters of fatty acids having 12 to 18 C atoms. Fatty acid partial esters of glycerol and sorbitol oxyethylates are also suitable solvents, preferably mono- and diesters of C₁₂-C₁₈ fatty acids and addition products of 20 to 60 moles ethylene with 1 mole glycerol or 1 mole sorbitol.

Mouth, tooth and/or denture care products according to the invention preferably comprise as solubilizing agents for the optionally included flavor oils, addition products of 20 to 60 moles ethylene oxide with hydrogenated or non-hydrogenated castor oil (namely with triglyceride of oxystearic or ricinoleic acid), with glycerol mono- and/or distearate or with sorbitol mono- and/or distearate.

Examples of other customary additives to mouth, tooth and denture care products include,
pigments, e.g. titanium dioxide, and/or colorings
pH adjusters and buffers, e.g. sodium bicarbonate, sodium citrate, sodium benzoate, citric acid, phosphoric acid or acid salts, e.g. NaH₂PO₄
wound-healing and anti-inflammatory substances, e.g. allantoin, urea, panthenol, azulene or camomile extract
other anti-tartar substances e.g. organophosphates such as hydroxyethane diphosphonate or azacycloheptane diphosphonate
preservatives, e.g. sorbic acid salts, p-hydroxybenzoic acid ester.
plaque inhibitors such as hexachlorophene, chlorhexidine, hexetidine, triclosan, bromchlorophene, phenylsalicylic acid ester.

In a specific embodiment, the composition is a mouthwash, a rinse, a denture cleaner or dental adhesive.

For preferred denture cleaners according to the invention, especially for denture-cleaning tablets and powder, in addition to the already listed ingredients for mouth, tooth and/or denture care products, per-compounds such as peroxyborate, peroxymonosulfate or percarbonate are also suitable. They have the advantage that in addition to a bleaching effect they simultaneously act to deodorize and/or disinfect. Such per-compounds are used in denture cleaners at between 0.01 and 10 weight %, and especially at between 0.5 and 5 weight %.

Enzymes, e.g. proteases and carbohydrase, are also suitable ingredients for the degradation of proteins and carbohydrates. The pH value can be in the range pH 4 to 12, and especially in the range pH 5 to pH 11.

Still other additives are required for denture cleaning tablets, for example materials that cause effervescence, e.g. CO₂-liberating substances such as sodium bicarbonate, fillers such as sodium sulfate or dextrose, lubricants, e.g. magnesium stearate, flow regulators such as colloidal silicon dioxide and granulating agents such as the already mentioned high-molecular-weight polyethylene glycols or polyvinyl pyrrolidone.

Denture fixative agents can be offered as powders, creams, films or liquids and they assist denture adhesion. Materials from natural and synthetic sources are suitable as the active agents. In addition to alginates, natural materials include plant gums such as gum arabic, traganth gum and karaya gum as well as natural rubber. Particular use is made of alginates and synthetic materials such as sodium carboxymethyl cellulose, high-molecular-weight ethylene oxide copolymers, salts of vinyl ether-maleic acid copolymer, and polyacrylamide.

Hydrophobic bases are particularly suitable as additives for paste and liquid products, especially hydrocarbons such as white vaseline (DAB—German Pharmacopoeia) or mineral oil.

Additional subjects of the invention are to be seen in suitable utilization possibilities for inventive bleaching systems and compositions.

Thus, one possibility of realization for the present invention is in the use of an above-described inventive bleaching system for the bleaching or disinfecting treatment of filter media, textiles, hard surfaces, furs, paper, hides or leather or for inhibiting color transfer when washing textiles.

A further possibility of realization for the present invention is the use of an above-described inventive composition for the bleaching or disinfecting treatment of filter media, textiles, hard surfaces, furs, paper, hides or leather or for inhibiting color transfer when washing textiles.

The previous embodiments are correspondingly valid for both subject matters. The application for inhibiting the color transfer when washing textiles is particularly based on the fact that during the wash cycle, detached colored materials are inventively bleached before they settle out on the washing and lead to unwanted color effects.

The following examples illustrate the invention in more detail, without limiting it in any way.

### EXAMPLES

### Example 1

Bleach Performance of the Combination of Oxidase and Perhydrolase

The bleach performance of the combination of oxidase and perhydrolase was investigated in a textile detergent with the following general formulation (matrix) listed in Table 1.

TABLE 1
General formulation for a textile detergent

| Chem. Name | wt. % pure substance |
|---|---|
| Xanthane Gum | 0.3-0.5 |
| Antifoaming agent | 0.2-0.4 |
| Glycerine | 6-7 |
| Ethanol | 0.3-0.5 |
| FAEOS | 4-7 |
| Non-ionic surfactant (FAEO. APG. | 24-28 |
| inter alia) | |
| Boric acid | 1 |
| Sodium citrate x 2H₂O | 1-2 |
| Caustic soda | 2-4 |
| Coconut fatty acid | 14-16 |
| HEDP | 0.5 |
| PVP | 0-0.4 |
| Optical brightener | 0-0.05 |
| Dye | 0-0.001 |
| Perfume | 0-2 |
| H₂O•demin. | Rest |

This formulation was dosed at 4.4 g/l, the pH being adjusted to 10 with soda during the described test.

Tea was used as the textile stain on cotton using the following preparations:
020 J Co, obtainable from wfk Testgewebe GmbH; Brüggen-Bracht, Germany,
E-167, obtainable from EMPA Testmaterialien AG (St. Gallen, Switzerland) and
a corresponding stain manufactured by Henkel KGaA (Düsseldorf, Germany).

Round test fabric pieces (diameter 10 mm) were incubated in a 24-well microtiter plate in 1 ml of detergent solution for 30 min at 37° C. with agitation at 100 rpm. The detergent solution contained 540 μg choline oxidase KC2 (SEQ ID NO. 2) and 200 mM choline chloride, as well as 10 μg of an invention relevant perhydrolase and 50 mM butyric acid, methyl ester (BSME). During the incubation, air was introduced into each test base by means of a needle with an internal diameter of 0.5 to 1 mm. Each test was carried out as a double determination against a doubly determined control with solely choline oxidase or perhydrolase with H₂O₂.

After washing, the degree of whiteness of the washed textiles was measured in comparison with a whiteness standard (d/8, Ø 8 mm, SCI/SCE), which had been set at 100% (L-value determination). The measurement was made using a colorimeter (Minolta Cm508d) with a light setting of 10°/D65. The results obtained are presented in the following table in terms of percent remission, i.e. as a percentage in comparison with the white standard together with the respective starting values. The bleaching power is given as ΔL, the difference in remission from the basic washing formulation without enzymes. The controls reflect the individual bleach activities of choline oxidase and perhydrolase with added H₂O₂.

The results are shown in Table 2:

TABLE 2
Determination of the bleaching power of an inventive enzymatic
bleaching system in the context of a textile detergent formulation

| Basic detergent with | Bleach power ΔL | Standard deviation |
|---|---|---|
| Oxidase | 2.43 | — |
| Oxidase + Perhydrolase | 3.75 | 0.10 |

The results show a significantly superior bleaching power of the inventive combination of oxidase and perhydrolase over the mere oxidase.

### Example 2

Sequence Comparisons

Various data exist in the relevant literature for choline oxidase from Arthrobacter globiformis, which illustrates the prior art for the choline oxidases that are particularly relevant to the invention. The first literature reference (hereinafter called “old”) found in connexion with the present invention is: Deshnium, P., Los, D. A., Hayashi, H., Mustardy, L., und Murata, N. (1995): “Transformation of Synechococcus with a gene for choline oxidase enhances tolerance to salt stress”, Plant Mol. Biol., volume 29, pages 897 to 907.

A new (corrected) data bank entry is visible at the National Center for Biotechnology Information NCBI, National Institutes of Health, Bethesda, Md., USA in the Internet under the address http://www.ncbi.nlm.nih.gov/entrez/viewer.fcgi?db=protein&val=31979241 (Fan F., Ghanem M., Gadda G.; “codA gene for choline oxidase from genomic DNA of Arthrobacter RT globiformis (ATCC 8010).” The degrees of homology of the choline oxidases from Arthrobacter are identified as shown in the following Table 3:

TABLE 3
Degrees of homology of the choline oxidases from Arthrobacter

| | COD | | | | COD | COD |
|---|---|---|---|---|---|---|
| | Arthrobacter | | | COD | Arthrobacter | Arthrobacter |
| | nicotianae | | COD | Arthrobacter | globiformis | globiformis |
| | (KC2) | KC2s | hybrid | aurescens | new | old |
| COD | [symbol] /100 | 98.8 | [symbol] | 73.3 | 76.7 | 76.4 |
| Arthrobacter | | | | | | |
| nicotianae | | | | | | |
| (KC2) | | | | | | |
| KC2s | 98.6 | 100/100 | 86.0 | 73.0 | 76.3 | 76.0 |
| COD hybrid | 86.3 | 86.3 | [symbol] /100 | 86.9 | 81.3 | 81.0 |
| COD | [symbol] | 75.9 | [symbol] | [symbol] /100 | 83.1 | 82.9 |
| Arthrobacter | | | | | | |
| aurescens | | | | | | |
| COD | [symbol] | 76.5 | [symbol] | [symbol] | [symbol] /100 | 99.5 |
| Arthrobacter | | | | | | |
| globiformis | | | | | | |
| new | | | | | | |
| COD | 61.0 | 61.0 | 67.9 | 71.4 | 80.9 | 100/100 |
| Arthrobacter | | | | | | |
| globiformis | | | | | | |
| old | | | | | | |

Key:
COD: Choline oxidase [symbol]
identical bases in % (DNA)
Normal print: identical amino acids in % (protein)

The choline oxidase from Arthrobacter globiformis, listed in the data bank of the NCBI under the numbers AAP68832 and AAS99880 exhibits the following homology values on the amino acid level to the particularly inventively relevant choline oxidases over each total length: To the choline oxidase from Arthrobacter nicotianiae (KC2; SEQ ID NO. 2) 77.7% identity, to the choline oxidase from A. aurescens (SEQ ID NO. 4) 89.6% identity, to the hybrid choline oxidase according to SEQ ID NO. 6 84.5% identity and to the N-terminal deleted choline oxidase from A. nicotianiae (KC2s; SEQ ID NO. 28) 78.5% identity.

The homologization leading to these values is as described in the description according to Lipman and Pearson with the help of the computer program Vector NTI® Suite 7.0, obtainable from InforMax Inc., Bethesda, USA carried out with the pre-defined standard parameters.

1. Enzymatic bleaching system comprising at least one oxidase and at least one perhydrolase.

2. Bleaching system according to claim 1, wherein the oxidase is selected from the group consisting of:
a) choline oxidases, whose amino acid sequence matches that listed in SEQ ID No. 2 to at least 76.5%;
b) choline oxidases, whose amino acid sequence matches that listed in SEQ ID No. 4 to at least 89%;
c) choline oxidases, whose amino acid sequence matches that listed in SEQ ID No. 6 to at least 83.8%;
d) choline oxidases, whose amino acid sequence matches that listed in SEQ ID No. 28 to at least 76.4%;
e) choline oxidases obtainable by one or multiple conservative amino acid exchanges from a choline oxidase selected from the group consisting of:
(i) choline oxidases, whose amino acid sequence matches that listed in SEQ ID No. 2 to at least 76.5%,
(ii) choline oxidases, whose amino acid sequence matches that listed in SEQ ID No. 4 to at least 89%,
(iii) choline oxidases, whose amino acid sequence matches that listed in SEQ ID No. 6 to at least 83.8%,
(iv) choline oxidases, whose amino acid sequence matches that listed in SEQ ID No. 28 to at least 76.4%;
f) choline oxidases obtainable by derivatization of a choline oxidase selected from the group consisting of:
(i) choline oxidases, whose amino acid sequence matches that listed in SEQ ID No. 2 to at least 76.5%,
(ii) choline oxidases, whose amino acid sequence matches that listed in SEQ ID No. 4 to at least 89%,
(iii) choline oxidases, whose amino acid sequence matches that listed in SEQ ID No. 6 to at least 83.8%,
(iv) choline oxidases, whose amino acid sequence matches that listed in SEQ ID No. 28 to at least 76.4%;
g) choline oxidases obtainable by fragmentation of a choline oxidase selected from the group consisting of:
(i) choline oxidases, whose amino acid sequence matches that listed in SEQ ID No. 2 to at least 76.5%,
(ii) choline oxidases, whose amino acid sequence matches that listed in SEQ ID No. 4 to at least 89%,
(iii) choline oxidases, whose amino acid sequence matches that listed in SEQ ID No. 6 to at least 83.8%,
(iv) choline oxidases, whose amino acid sequence matches that listed in SEQ ID No. 28 to at least 76.4%;
h) choline oxidases obtainable by deletion mutation of a choline oxidase selected from the group consisting of:
(i) choline oxidases, whose amino acid sequence matches that listed in SEQ ID No. 2 to at least 76.5%,
(ii) choline oxidases, whose amino acid sequence matches that listed in SEQ ID No. 4 to at least 89%,
(iii) choline oxidases, whose amino acid sequence matches that listed in SEQ ID No. 6 to at least 83.8%,
(iv) choline oxidases, whose amino acid sequence matches that listed in SEQ ID No. 28 to at least 76.4%; and
i) choline oxidases obtainable by insertion mutation of a choline oxidase selected from the group consisting of:
(i) choline oxidases, whose amino acid sequence matches that listed in SEQ ID No. 2 to at least 76.5%,
(ii) choline oxidases, whose amino acid sequence matches that listed in SEQ ID No. 4 to at least 89%,
(iii) choline oxidases, whose amino acid sequence matches that listed in SEQ ID No. 6 to at least 83.8%,
(iv) choline oxidases, whose amino acid sequence matches that listed in SEQ ID No. 28 to at least 76.4%.

3. Bleaching system according to claim 1, wherein the perhydrolase is selected from
a) perhydrolases, whose amino acid sequence corresponds to that listed in SEQ ID NO. 26, but carries at least one of exchanged amino acids at the sequence positions selected from 11, 15, 21, 38, 50, 54, 58, 77, 83, 89, 93, 96, 107, 117, 120, 134, 135, 136, 140, 147, 150, 154, 155, 160, 161, 171, 179, 180, 181, 194, 205, 208, 213, 216, 217, 238, 239, 251, 253, 257, 261,
b) perhydrolases, whose amino acid sequence corresponds to that listed in SEQ ID NO. 26, but carries at least one of exchanged amino acids at the sequence positions selected from 11, 58, 77, 89, 96, 117, 120, 134, 135, 136, 140, 147, 150, 161, 208, 216, 217, 238,
c) perhydrolases, whose amino acid sequence corresponds to that listed in SEQ ID NO. 26, but carries at least one of exchanged amino acids at the sequence positions selected from 58, 89, 96, 117, 216, 217,
d) perhydrolases, whose amino acid sequence corresponds to that listed in SEQ ID NO. 26, but exhibits at least one of amino acid exchanges T58A or T58Q, L89S, N96D, G117D, L216W and N217D,
e) perhydrolases, whose amino acid sequence matches the amino acid sequences listed in the SEQ ID NO. 8, 10, 12, 14, 16, 18, 20, 22 or 24 to at least 70%.

4. Bleaching system according to claim 2, wherein the perhydrolase is selected from
a) perhydrolases, whose amino acid sequence corresponds to that listed in SEQ ID NO. 26, but carries at least one of exchanged amino acids at the sequence positions selected from 11, 15, 21, 38, 50, 54, 58, 77, 83, 89, 93, 96, 107, 117, 120, 134, 135, 136, 140, 147, 150, 154, 155, 160, 161, 171, 179, 180, 181, 194, 205, 208, 213, 216, 217, 238, 239, 251, 253, 257, 261,
b) perhydrolases, whose amino acid sequence corresponds to that listed in SEQ ID NO. 26, but carries at least one of exchanged amino acids at the sequence positions selected from 11, 58, 77, 89, 96, 117, 120, 134, 135, 136, 140, 147, 150, 161, 208, 216, 217, 238,
c) perhydrolases, whose amino acid sequence corresponds to that listed in SEQ ID NO. 26, but carries at least one of exchanged amino acids at the sequence positions selected from 58, 89, 96, 117, 216, 217,
d) perhydrolases, whose amino acid sequence corresponds to that listed in SEQ ID NO. 26, but exhibits at least one of amino acid exchanges T58A or T58Q, L89S, N96D, G117D, L216W and N217D,
e) perhydrolases, whose amino acid sequence matches the amino acid sequences listed in the SEQ ID NO. 8, 10, 12, 14, 16, 18, 20, 22 or 24 to at least 70%.

5. A composition according to claim 1, the composition being selected from the group consisting of body care compositions, hair shampoos, hair care compositions, mouth-, tooth- or denture care compositions, care compositions for tooth braces, cosmetics, therapeutics, textile detergents, cleaning compositions, rinse agents, textile detergents for washing machines, detergents for hand washing, hand dish washing detergents, automatic dishwasher detergents, disinfectants and compositions for bleaching or disinfecting filter media, textiles, furs/pelts, paper, hides or leather.

6. Composition according to claim 5, wherein said composition is selected from the group consisting of textile detergents, bleaching agents and cleaning compositions.

7. Composition according to claim 6 wherein the composition is selected from the group consisting of detergents for machine washing textiles and automatic dishwasher detergents.

8. Composition according to claim 1 exhibiting an oxidase activity of 1 to 20 000 U/g, preferably 10 to 10 000 U/g, particularly preferably 100 to 1000 U/g and a perhydrolase concentration of 0.5 to 100 μg/ml, preferably 1 to 75 μg/ml, particularly preferably 10 to 50 μg/ml.

9. Composition according to claim 5 wherein said composition is present as a free-flowing powder with a bulk density of 300 to 1200 g/l, preferably 400 to 1000 g/l, particularly preferably 500 to 900 g/l.

10. Composition according to claim 5 wherein said composition is present in the form of a pasty or liquid detergent.

11. Composition according to claim 9 additionally comprising:
5 wt. % to 70 wt. % surfactant,
10 wt. % to 65 wt. % of water-soluble, water-dispersible inorganic builder,
1 wt. % to 10 wt. % of water-soluble organic builders,
not more than 15 wt. % of a solid composition selected from inorganic acid, organic acids, salts or inorganic acids, and salts of organic acids,
not more than 5 wt. % sequestrants for heavy metals,
not more than 5 wt. % graying inhibitor,
not more than 5 wt. % color transfer inhibitor and
not more than 5 wt. % foam inhibitor.

12. Composition according to claim 10 additionally comprising:
5 wt. % to 70 wt. % surfactant,
10 wt. % to 65 wt. % of water-soluble, water-dispersible inorganic builder,
1 wt. % to 10 wt. % of water-soluble organic builders,
not more than 15 wt. % of a solid composition selected from inorganic acid, organic acids, salts or inorganic acids, and salts of organic acids,
not more than 5 wt. % sequestrants for heavy metals,
not more than 5 wt. % graying inhibitor,
not more than 5 wt. % color transfer inhibitor and
not more than 5 wt. % foam inhibitor.

13. Composition according to claim 1 additionally comprising at least one additional enzyme selected from the group consisting of proteases, amylases, cellulases, hemicellulases, further oxidoreductases and lipases.

14. A process for treating a substrate, the process comprising:
(a) providing a substrate selected from the group consisting of filter media, textiles, hard surfaces, furs/pelts, paper, hides and leather;
(b) providing an enzymatic bleaching system according to claim 1; and
(c) applying the bleaching system to the substrate.

15. A process for treating a substrate, the process comprising:
(a) providing a substrate selected from the group consisting of filter media, textiles, hard surfaces, furs/pelts, paper, hides and leather;
(b) providing an enzymatic bleaching system according to claim 2; and
(c) applying the bleaching system to the substrate.

16. A process for treating a substrate, the process comprising:
(a) providing a substrate selected from the group consisting of filter media, textiles, hard surfaces, furs/pelts, paper, hides and leather;
(b) providing an enzymatic bleaching system according to claim 3; and
(c) applying the bleaching system to the substrate.

17. A process for treating a substrate, the process comprising:
(a) providing a substrate selected from the group consisting of filter media, textiles, hard surfaces, furs/pelts, paper, hides and leather;
(b) providing an enzymatic bleaching system according to claim 4; and
(c) applying the bleaching system to the substrate.


SEQUENCE LISTING (28 sequences)

SEQ ID NO: 1 - DNA, 1629 bp, Arthrobacter nicotianiae (DSMZ-ID 96-878)
  gene (1)..(1629): Choline oxidase (KC2)
  CDS (1)..(1626)
acgagaaagggcgaaaaattgaacattgaaaagaaggacttcgactacattgtgatcggcggcggttctgccggcgctgcggtggcttctcgcctgagcgaggatccatcggtgagtgttgcactggttgaagcaggtcccgacgaccgcggctacgatgaagtgctgcagctggaccgctggatggaactgctggaatccggcttggactgggactacccgatcgaaccgcaggaaaacggcaactcgttcatgcgccatgcccgtgccaaggtcatgggcggctgctccagccacaactcctgcatcgccttctgggcgccgcgtgaggaccttgacgagtgggagtcgaagttcggcgccaccggctggaactccgagatggcctaccgcctgtacaagaagctggaaaccaatgaggacgccggaccggatgcaccgcaccacggcgactcgggcccggtcaagctgatgaacgtgccgccggtggacccctgcggcgtggcaatcctggacgccgccgagcaggcaggcatcccgcgcgccaagttcaacaacaacgagacggtcatcaacggcgcgaacttcttccagatcaaccgcctgccggacggcacccgtgcctcctcctcggtctcctacatccacccgatcgagggacgcgagaacttcttcctgctcaccggactgcaggcgcgcaagctgaacttcgacgccgacaagcgctgcaccggcgtggacgtggtggacggggccttcggccgcaccgtgacgctgaacgccgcccgcgaggtcgtcgtctccgccggcgccattgactcgccgaagctgctgatgctctccggcatcggacctgccgaacacctcaaggaggtcggcgtcgaggtgcttgtcgacgctccaggcgtgggcgaacacctgcaggaccacccggaaggcgtgatccagtgggaggctaagaagccgatggttgaaacctccacccagtggtgggaaatcggcatcttcgcaccgacccaggaaggcctcgaccgtccggatctgatgatgcactacggttcggtgcccttcgacatgcacaccctgcgttggggttacccgaccagcgagaacaccttctgcctgaccccgaatgtcacccatgcgaagtcgcgcggcaccgtgcgcctgcgcagctgcgacttcagcgacaagccgaaggtcgaccctcgctacttcaccgatccagagggccacgacgcccgggtcatgaccttcggcatccgcaaggcccgcgagatcgtcgcccagtcgccgatggcagagtgggctggcgaggagcagttcccgggcaaggacgtgcagactgacgaagagatcttcgactacctgcgccgctgccacaacactgtctaccacccggctggttcggtgcgcatgggcgccgaggacgatgtgatgagcccgctggatccgcagctgcgggtcaagggagtctcgggcctgcgcgtggccgacgcttcggtgatgccggagctggtcaccgtgaacccgaacatcacggtgatgatgatcggtgaacgctgcgccgagctgattcaggaaaaccctgtggaagcccgccagagcgaactggttagcgaagcctag

SEQ ID NO: 2 - protein, 542 aa, Arthrobacter nicotianiae (DSMZ-ID 96-878)
TRKGEKLNIEKKDFDYIVIGGGSAGAAVASRLSEDPSVSVALVEAGPDDRGYDEVLQLDRWMELLESGLDWDYPIEPQENGNSFMRHARAKVMGGCSSHNSCIAFWAPREDLDEWESKFGATGWNSEMAYRLYKKLETNEDAGPDAPHHGDSGPVKLMNVPPVDPCGVAILDAAEQAGIPRAKFNNNETVINGANFFQINRLPDGTRASSSVSYIHPIEGRENFFLLTGLQARKLNFDADKRCTGVDVVDGAFGRTVTLNAAREVVVSAGAIDSPKLLMLSGIGPAEHLKEVGVEVLVDAPGVGEHLQDHPEGVIQWEAKKPMVETSTQWWEIGIFAPTQEGLDRPDLMMHYGSVPFDMHTLRWGYPTSENTFCLTPNVTHAKSRGTVRLRSCDFSDKPKVDPRYFTDPEGHDARVMTFGIRKAREIVAQSPMAEWAGEEQFPGKDVQTDEEIFDYLRRCHNTVYHPAGSVRMGAEDDVMSPLDPQLRVKGVSGLRVADASVMPELVTVNPNITVMMIGERCAELIQENPVEARQSELVSEA

SEQ ID NO: 3 - DNA, 1650 bp, Arthrobacter aurescens (ATCC 13344)
  gene (1)..(1650): Choline oxidase
  CDS (1)..(1650)
atgcacgtcgacaatattgagaacctcagcgagcgcgggttcgattacatcgtcatcggcggaggatctgccggggctgccgtcgccgcccggctgagcgaggaccccgccgttaccgttgccctggtggaagccggcccggatgaccggaacctcccggaaatcctgcaattggaccggtggatggaactgctggaatcggggtacgactgggactacccggtggaaccgcaggagaacggcaactcattcatgcgccacgcccgcgccaaggtgatgggcggctgttccagccacaattcctgcatcgctttctgggctccccgggaggacctggatgagtgggagtccaagtacggtgccaccggttggaacgctgccgcggcctggcctttgtatcagcgcttggaaagcaacgaggacgctggcccggatgccccacatcacggccatgatggtccggtgcacctgatgaacgttcctccgaatgatccggccggggttgcgctcctggacgcctgtgagcagagcggcattccccgggcaaagttcaacgacggcaccacagtgatcaacggcgctaatttcttccagatcaaccgccgttcggacggtacgcgttcctccagttctgtttcctacattcaccccatcatggctcgcgaaaacttcacgctgttgaccggcctgcgtgcccgccaactggtgttcgacgcggacaagcgttgcaccggcgtcgacgtcgttgactctgccttcggcaggacacacaggctcaccgcgcagcgcgaggtcatcctctccacgggcgccattgattcccccaagctgctcatgctttccggaatcggcccggccgagcacctggctcaacatggcatcgaggtggtggtggactcccccggtgttggcgagcatctgcaggaccacccggagggcgtggtgcagttcgaggccaagcagcccatggttcagtcgtccacccaatggtgggagatcggcatcttcactcccaccgaggagggactggaccgcccggacctgatgatgcactacggctccgtgccgttcgacatgaacaccctccgctacggttaccccaccacggagaacggcttcagccttaccccgaacgtcacgcacgcccgttcccgcgggaccgtccgtttgcgcagcagggacttccgcgacaagcccatggtggacccgcggtacttcaccgatcccgaggggcacgacatgcgcgtcatggtggccggcatccgcaaggcccgggaaatcgcggctcagccggccatggccgagtggaccggacgggagctctcccccggaatcgaggcccagactgatgaggagcttcaggactacatccgcaaaacccacaacaccgtctaccaccccgtcggtaccgtccgcatgggcccggtggaggaccccatgtctcccttggatccggagctgcgtgtcaaaggcgtcagcggcctacgggtggcagacgcctccgtgatgcccgaacacgtcacagtgaaccccaacatcaccgtgatgatgatcggcgagcgctgtgctgatctcatcaaagcccggctgggtgccagccgggtggaggaaaccacgacgacggaggcggacttcagcttgtcccacgcc

SEQ ID NO: 4 - protein, 550 aa, Arthrobacter aurescens (ATCC 13344)
MHVDNIENLSERGFDYIVIGGGSAGAAVAARLSEDPAVTVALVEAGPDDRNLPEILQLDRWMELLESGYDWDYPVEPQENGNSFMRHARAKVMGGCSSHNSCIAFWAPREDLDEWESKYGATGWNAAAAWPLYQRLESNEDAGPDAPHHGHDGPVHLMNVPPNDPAGVALLDACEQSGIPRAKFNDGTTVINGANFFQINRRSDGTRSSSSVSYIHPIMARENFTLLTGLRARQLVFDADKRCTGVDVVDSAFGRTHRLTAQREVILSTGAIDSPKLLMLSGIGPAEHLAQHGIEVVVDSPGVGEHLQDHPEGVVQFEAKQPMVQSSTQWWEIGIFTPTEEGLDRPDLMMHYGSVPFDMNTLRYGYPTTENGFSLTPNVTHARSRGTVRLRSRDFRDKPMVDPRYFTDPEGHDMRVMVAGIRKAREIAAQPAMAEWTGRELSPGIEAQTDEELQDYIRKTHNTVYHPVGTVRMGPVEDPMSPLDPELRVKGVSGLRVADASVMPEHVTVNPNITVMMIGERCADLIKARLGASRVEETTTTEADFSLSHA

SEQ ID NO: 5 - DNA, 1650 bp, Artificial sequence
  Hybrid choline oxidase
  gene (1)..(1650): Choline oxidase
  CDS (1)..(1650)
atgcacgtcgacaatattgagaacctcagcgagcgcgggttcgattacatcgtcatcggcggaggatctgccggggctgccgtcgccgcccggctgagcgaggaccccgccgttaccgttgccctggtggaagccggcccggatgaccggaacctcccggaaatcctgcaattggaccggtggatggaactgctggaatcggggtacgactgggactacccggtggaaccgcaggagaacggcaactcattcatgcgccacgcccgcgccaaggtcatgggcggctgctccagccacaactcctgcatcgccttctgggcgccgcgtgaggaccttgacgagtgggagtcgaagttcggcgccaccggctggaactccgagatggcctaccgcctgtacaagaagctggaaaccaatgaggacgccggaccggatgcaccgcaccacggcgactcgggcccggtcaagctgatgaacgtgccgccggtggacccctgcggcgtggcaatcctggacgccgccgagcaggcaggcatcccgcgcgccaagttcaacaacaacgagacggtcatcaacggcgcgaacttcttccagatcaaccgcctgccggacggcacccgtgcctcctcctcggtctcctacatccacccgatcgagggacgcgagaacttcttcctgctcaccggactgcaggcgcgcaagctgaacttcgacgccgacaagcgctgcaccggcgtggacgtggtggacggggccttcggccgcaccgtgacgctgaacgccgcccgcgaggtcgtcgtctccgccggcgccattgactcgccgaagctgctgatgctctccggcatcggacctgccgaacacctcaaggaggtcggcgtcgaggtgcttgtcgacgctccaggcgtgggcgaacacctgcaggaccacccggaaggcgtgatccagtgggaggctaagaagccgatggttgaaacctccacccagtggtgggaaatcggcatcttcgcaccgacccaggaaggcctcgaccgtccggatctgatgatgcactacggttcggtgcccttcgacatgcacaccctgcgttggggttacccgaccagcgagaacaccttctgcctgaccccgaatgtcacccatgcgaagtcgcgcggcaccgtgcgcctgcgcagcagggacttccgcgacaagcccatggtggacccgcggtacttcaccgatcccgaggggcacgacatgcgcgtcatggtggccggcatccgcaaggcccgggaaatcgcggctcagccggccatggccgagtggaccggacgggagctctcccccggaatcgaggcccagactgatgaggagcttcaggactacatccgcaaaacccacaacaccgtctaccaccccgtcggtaccgtccgcatgggcccggtggaggaccccatgtctcccttggatccggagctgcgtgtcaaaggcgtcagcggcctacgggtggcagacgcctccgtgatgcccgaacacgtcacagtgaaccccaacatcaccgtgatgatgatcggcgagcgctgtgctgatctcatcaaagcccggctgggtgccagccgggtggaggaaaccacgacgacggaggcggacttcagcttgtcccacgcc

SEQ ID NO: 6 - protein, 550 aa, Artificial sequence
  Hybrid choline oxidase
MHVDNIENLSERGFDYIVIGGGSAGAAVAARLSEDPAVTVALVEAGPDDRNLPEILQLDRWMELLESGYDWDYPVEPQENGNSFMRHARAKVMGGCSSHNSCIAFWAPREDLDEWESKFGATGWNSEMAYRLYKKLETNEDAGPDAPHHGDSGPVKLMNVPPVDPCGVAILDAAEQAGIPRAKFNNNETVINGANFFQINRLPDGTRASSSVSYIHPIEGRENFFLLTGLQARKLNFDADKRCTGVDVVDGAFGRTVTLNAAREVVVSAGAIDSPKLLMLSGIGPAEHLKEVGVEVLVDAPGVGEHLQDHPEGVIQWEAKKPMVETSTQWWEIGIFAPTQEGLDRPDLMMHYGSVPFDMHTLRWGYPTSENTFCLTPNVTHAKSRGTVRLRSRDFRDKPMVDPRYFTDPEGHDMRVMVAGIRKAREIAAQPAMAEWTGRELSPGIEAQTDEELQDYIRKTHNTVYHPVGTVRMGPVEDPMSPLDPELRVKGVSGLRVADASVMPEHVTVNPNITVMMIGERCADLIKARLGASRVEETTTTEADFSLSHA

SEQ ID NO: 7 - DNA, 824 bp, Artificial sequence
  Variant L89S/L216W/N217D of Subtilisin Carlsberg
  gene (1)..(824): Variant L89S/L216W/N217D of Subtilisin Carlsberg
  CDS (1)..(822)
gcgcaaaccgttccttacggcattcctctcattaaagcggacaaagtgcaggctcaaggctttaagggagcgaatgtaaaagtagccgtcctggatacaggaatccaagcttctcatccggacttgaacgtagtcggcggagcaagctttgtggctggcgaagcttataacaccgacggcaacggacacggcacacatgttgccggtacagtagctgcgcttgacaatacaacgggtgtattaggcgttgcgccaagcgtatcctcgtacgcggttaaagtactgaattcaagcggaagcggatcatacagcggcattgtaagcggaatcgagtgggcgactacaaacggcatggatgttatcaatatgagccttgggggagcatcaggctcgacagcgatgaaacaggcagtcgacaatgcatatgcaagaggggttgtcgttgtagctgcagcagggaacagcggatcttcaggaaacacgaatacaattggctatcctgcgaaatacgattctgtcatcgctgttggtgcggtagactctaacagcaacagagcttcattttccagcgtcggagcagagcttgaagtcatggctcctggcgcaggcgtatacagcacttacccaacgaacacttatgcaacatgggacggaacgtcaatggcttctcctcatgtagcgggagcagcagctttgatcttgtcaaaacatccgaacctttcagcttcacaagtccgcaaccgtctctccagcacggcgacttatttgggaagctccttctactatgggaaaggtctgatcaatgtcgaagctgccgctcaata

SEQ ID NO: 8 - protein, 274 aa, Artificial sequence
  Variant L89S/L216W/N217D of Subtilisin Carlsberg
AQTVPYGIPLIKADKVQAQGFKGANVKVAVLDTGIQASHPDLNVVGGASFVAGEAYNTDGNGHGTHVAGTVAALDNTTGVLGVAPSVSSYAVKVLNSSGSGSYSGIVSGIEWATTNGMDVINMSLGGASGSTAMKQAVDNAYARGVVVVAAAGNSGSSGNTNTIGYPAKYDSVIAVGAVDSNSNRASFSSVGAELEVMAPGAGVYSTYPTNTYATWDGTSMASPHVAGAAALILSKHPNLSASQVRNRLSSTATYLGSSFYYGKGLINVEAAAQ

SEQ ID NO: 9 - DNA, 824 bp, Artificial sequence
  Variant L216W/N217D of Subtilisin Carlsberg
  gene (1)..(824): Variant L216W/N217D of Subtilisin Carlsberg
  CDS (1)..(822)
gcgcaaaccgttccttacggcattcctctcattaaagcggacaaagtgcaggctcaaggctttaagggagcgaatgtaaaagtagccgtcctggatacaggaatccaagcttctcatccggacttgaacgtagtcggcggagcaagctttgtggctggcgaagcttataacaccgacggcaacggacacggcacacatgttgccggtacagtagctgcgcttgacaatacaacgggtgtattaggcgttgcgccaagcgtatccttgtacgcggttaaagtactgaattcaagcggaagcggatcatacagcggcattgtaagcggaatcgagtgggcgactacaaacggcatggatgttatcaatatgagccttgggggagcatcaggctcgacagcgatgaaacaggcagtcgacaatgcatatgcaagaggggttgtcgttgtagctgcagcagggaacagcggatcttcaggaaacacgaatacaattggctatcctgcgaaatacgattctgtcatcgctgttggtgcggtagactctaacagcaacagagcttcattttccagcgtcggagcagagcttgaagtcatggctcctggcgcaggcgtatacagcacttacccaacgaacacttatgcaacatgggacggaacgtcaatggcttctcctcatgtagcgggagcagcagctttgatcttgtcaaaacatccgaacctttcagcttcacaagtccgcaaccgtctctccagcacggcgacttatttgggaagctccttctactatgggaaaggtctgatcaatgtcgaagctgccgctcaata

SEQ ID NO: 10 - protein, 274 aa, Artificial sequence
  Variant L216W/N217D of Subtilisin Carlsberg
AQTVPYGIPLIKADKVQAQGFKGANVKVAVLDTGIQASHPDLNVVGGASFVAGEAYNTDGNGHGTHVAGTVAALDNTTGVLGVAPSVSLYAVKVLNSSGSGSYSGIVSGIEWATTNGMDVINMSLGGASGSTAMKQAVDNAYARGVVVVAAAGNSGSSGNTNTIGYPAKYDSVIAVGAVDSNSNRASFSSVGAELEVMAPGAGVYSTYPTNTYATWDGTSMASPHVAGAAALILSKHPNLSASQVRNRLSSTATYLGSSFYYGKGLINVEAAAQ

SEQ ID NO: 11 - DNA, 824 bp, Artificial sequence
  Variant T58A/L89S/L216W/N217D of Subtilisin Carlsberg
  gene (1)..(824): Variant T58A/L89S/L216W/N217D of Subtilisin Carlsberg
  CDS (1)..(822)
gcgcaaaccgttccttacggcattcctctcattaaagcggacaaagtgcaggctcaaggctttaagggagcgaatgtaaaagtagccgtcctggatacaggaatccaagcttctcatccggacttgaacgtagtcggcggagcaagctttgtggctggcgaagcttataacgccgacggcaacggacacggcacacatgttgccggtacagtagctgcgcttgacaatacaacgggtgtattaggcgttgcgccaagcgtatcctcgtacgcggttaaagtactgaattcaagcggaagcggatcatacagcggcattgtaagcggaatcgagtgggcgacaacaaacggcatggatgttatcaatatgagccttgggggagcatcaggctcgacagcgatgaaacaggcagtcgacaatgcatatgcaagaggggtcgtcgttgtagctgcagcagggaacagcggatcttcaggaaacacgaatacaattggctatcctgcgaaatacgattctgtcatcgctgttggtgcggtagactctaacagcaacagagcttcattttccagcgtcggagcagagcttgaagtcatggctcctggcgcaggcgtatacagcacttacccaacgaacacttatgcaacatgggacggaacgtcaatggcttctcctcatgtagcgggagcagcagctttgatcttgtcaaaacatccgaacctttcagcttcacaagtccgcaaccgtctctccagcacggcgacttatttgggaagctccttctactatgggaaaggtctgatcaatgtcgaagctgccgctcaata

SEQ ID NO: 12 - protein, 274 aa, Artificial sequence
  Variant T58A/L89S/L216W/N217D of Subtilisin Carlsberg
AQTVPYGIPLIKADKVQAQGFKGANVKVAVLDTGIQASHPDLNVVGGASFVAGEAYNADGNGHGTHVAGTVAALDNTTGVLGVAPSVSSYAVKVLNSSGSGSYSGIVSGIEWATTNGMDVINMSLGGASGSTAMKQAVDNAYARGVVVVAAAGNSGSSGNTNTIGYPAKYDSVIAVGAVDSNSNRASFSSVGAELEVMAPGAGVYSTYPTNTYATWDGTSMASPHVAGAAALILSKHPNLSASQVRNRLSSTATYLGSSFYYGKGLINVEAAAQ

SEQ ID NO: 13 - DNA, 824 bp, Artificial sequence
  Variant T58A/G117D/L216W/N217D of Subtilisin Carlsberg
  gene (1)..(824): Variant T58A/G117D/L216W/N217D of Subtilisin Carlsberg
  CDS (1)..(822)
gcgcaaaccgttccttacggcattcctctcattaaagcggacaaagtgcaggctcaaggctttaagggagcgaatgtaaaagtagccgtcctggatacaggaatccaagcttctcatccggacttgaacgtagtcggcggagcaagctttgtggctggcgaagcttataacgccgacggcaacggacacggcacacatgttgccggtacagtagctgcgcttgacaatacaacgggtgtattaggcgttgcgccaagcgtatccttgtacgcggttaaagtactgaattcaagcggaagcggatcatacagcggcattgtaagcggaatcgagtgggcgacaacaaacgacatggatgttatcaatatgagccttgggggagcatcaggctcgacagcgatgaaacaggcagtcgacaatgcatatgcaagaggggtcgtcgttgtagctgcagcagggaacagcggatcttcaggaaacacgaatacaattggctatcctgcgaaatacgattctgtcatcgctgttggtgcggtagactctaacagcaacagagcttcattttccagcgtcggagcagagcttgaagtcatggctcctggcgcaggcgtatacagcacttacccaacgaacacttatgcaacatgggacggaacgtcaatggcttctcctcatgtagcgggagcagcagctttgatcttgtcaaaacatccgaacctttcagcttcacaagtccgcaaccgtctctccagcacggcgacttatttgggaagctccttctactatgggaaaggtctgatcaatgtcgaagctgccgctcaata

SEQ ID NO: 14 - protein, 274 aa, Artificial sequence
  Variant T58A/G117D/L216W/N217D of Subtilisin Carlsberg
AQTVPYGIPLIKADKVQAQGFKGANVKVAVLDTGIQASHPDLNVVGGASFVAGEAYNADGNGHGTHVAGTVAALDNTTGVLGVAPSVSLYAVKVLNSSGSGSYSGIVSGIEWATTNDMDVINMSLGGASGSTAMKQAVDNAYARGVVVVAAAGNSGSSGNTNTIGYPAKYDSVIAVGAVDSNSNRASFSSVGAELEVMAPGAGVYSTYPTNTYATWDGTSMASPHVAGAAALILSKHPNLSASQVRNRLSSTATYLGSSFYYGKGLINVEAAAQ

SEQ ID NO: 15 - DNA, 822 bp, Artificial sequence
  Variant T58A/L89S/L216W of Subtilisin Carlsberg
  gene (1)..(822): Variant T58A/L89S/L216W of Subtilisin Carlsberg
  CDS (1)..(822)
gcgcaaaccgttccttacggcattcctctcattaaagcggacaaagtgcaggctcaaggctttaagggagcgaatgtaaaagtagccgtcctggatacaggaatccaagcttctcatccggacttgaacgtagtcggcggagcaagctttgtggctggcgaagcttataacgccgacggcaacggacacggcacacatgttgccggtacagtagctgcgcttgacaatacaacgggtgttttaggcgttgcgccaagcgtatcctcgtacgcggttaaagtactgaattcaagcggaagcggatcatacagcggcattgtaagcggaatcgagtgggcgacaacaaacggcatggatgttatcaatatgagccttgggggagcatcaggctcgacagcgatgaaacaggcagtcgacaatgcatatgcaagaggggtcgtcgttgtagctgcagcagggaacagcggatcttcaggaaacacgaatacaattggctatcctgcgaaatacgattctgtcatcgctgttggtgcggtagactctaacagcaacagagcttcattttccagcgtcggagcagagcttgaagtcatggctcctggcgcaggcgtatacagcacttacccaacgaacacttatgcaacatggaacggaacgtcaatggcttctcctcatgtagcgggagcagcagctttgatcttgtcaaaacatccgaacctttcagcttcacaagtccgcaaccgtctctccagcacggcgacttatttgggaagctccttctactatgggaaaggtctgatcaatgtcgaagctgccgctcaa

SEQ ID NO: 16 - protein, 274 aa, Artificial sequence
  Variant T58A/L89S/L216W of Subtilisin Carlsberg
AQTVPYGIPLIKADKVQAQGFKGANVKVAVLDTGIQASHPDLNVVGGASFVAGEAYNADGNGHGTHVAGTVAALDNTTGVLGVAPSVSSYAVKVLNSSGSGSYSGIVSGIEWATTNGMDVINMSLGGASGSTAMKQAVDNAYARGVVVVAAAGNSGSSGNTNTIGYPAKYDSVIAVGAVDSNSNRASFSSVGAELEVMAPGAGVYSTYPTNTYATWNGTSMASPHVAGAAALILSKHPNLSASQVRNRLSSTATYLGSSFYYGKGLINVEAAAQ

SEQ ID NO: 17 - DNA, 824 bp, Artificial sequence
  Variant T58A/L89S/N96D/L216W of Subtilisin Carlsberg
  gene (1)..(824): Variant T58A/L89S/N96D/L216W of Subtilisin Carlsberg
  CDS (1)..(822)
gcgcagaccgttccttacggcattcctctcattaaagcggacaaagtgcaggctcaaggctttaagggagcgaatgtaaaagtagccgtcctggatacaggaatccaggcttctcatccggacttgaacgtagtcggcggagcaagctttgtggctggcgaagcttataacgccgacggcaacggacacggcacacatgttgccggtacagtagctgcgctcgacaatacaacgggtgttttaggcgttgcgccaagcgtatcctcgtacgcggttaaagtactggattcaagcggaagcggatcatacagcggcattgtaagcggaatcgagtgggcgacaacaaacggcatggatgttatcaatatgagccttgggggagcatcaggctcgacagcgatgaaacaggcagtcgacaatgcatatgcgagaggggtcgtcgttgtagctgcagcagggaacagcggatcttcaggaaacacgaatacaattggctatcctgcgaaatacgattctgtcatcgctgttggtgcggtagactctaacagcaacagagcttcattttccagcgtcggagcagagcttgaagtcatggctcctggcgcaggcgtatacagcacttacccaacgaacacttatgcaacatggaacggaacgtcaatggcttctcctcatgtagcgggagcagcagctttgatcttgtcaaaacatccgaacctttcagcttcacaagtccgcaaccgtctctccagcacggcgacttatttgggaagctccttctactatgggaaaggtctgatcaatgtcgaagctgccgctcaata

SEQ ID NO: 18 - protein, 274 aa, Artificial sequence
  Variant T58A/L89S/N96D/L216W of Subtilisin Carlsberg
AQTVPYGIPLIKADKVQAQGFKGANVKVAVLDTGIQASHPDLNVVGGASFVAGEAYNADGNGHGTHVAGTVAALDNTTGVLGVAPSVSSYAVKVLDSSGSGSYSGIVSGIEWATTNGMDVINMSLGGASGSTAMKQAVDNAYARGVVVVAAAGNSGSSGNTNTIGYPAKYDSVIAVGAVDSNSNRASFSSVGAELEVMAPGAGVYSTYPTNTYATWNGTSMASPHVAGAAALILSKHPNLSASQVRNRLSSTATYLGSSFYYGKGLINVEAAAQ

SEQ ID NO: 19 - DNA, 824 bp, Artificial sequence
  Variant T58A/L216W of Subtilisin Carlsberg
  gene (1)..(824): Variant T58A/L216W of Subtilisin Carlsberg
  CDS (1)..(822)
gcgcaaaccgttccttacggcattcctctcattaaagcggacaaagtgcaggctcaaggctttaagggagcgaatgtaaaagtagccgtcctggatacaggaatccaagcttctcatccggacttgaacgtagtcggcggagcaagctttgtggctggcgaagcttataacgccgacggcaacggacacggcacacatgttgccggtacagtagctgcgcttgacaatacaacgggtgttttaggcgttgcgccaagcgtatccttgtacgcggttaaagtactgaattcaagcggaagcggatcatacagcggcattgtaagcggaatcgagtgggcgacaacaaacggcatggatgttatcaatatgagccttgggggagcatcaggctcgacagcgatgaaacaggcagtcgacaatgcatatgcaagaggggtcgtcgttgtagctgcagcagggaacagcggatcttcaggaaacacgaatacaattggctatcctgcgaaatacgattctgtcatcgctgttggtgcggtagactctaacagcaacagagcttcattttccagcgtcggagcagagcttgaagtcatggctcctggcgcaggcgtatacagcacttacccaacgaacacttatgcaacatggaacggaacgtcaatggcttctcctcatgtagcgggagcagcagctttgatcttgtcaaaacatccgaacctttcagcttcacaagtccgcaaccgtctctccagcacggcgacttatttgggaagctccttctactatgggaaaggtctgatcaatgtcgaagctgccgctcaata

SEQ ID NO: 20 - protein, 274 aa, Artificial sequence
  Variant T58A/L216W of Subtilisin Carlsberg
AQTVPYGIPLIKADKVQAQGFKGANVKVAVLDTGIQASHPDLNVVGGASFVAGEAYNADGNGHGTHVAGTVAALDNTTGVLGVAPSVSLYAVKVLNSSGSGSYSGIVSGIEWATTNGMDVINMSLGGASGSTAMKQAVDNAYARGVVVVAAAGNSGSSGNTNTIGYPAKYDSVIAVGAVDSNSNRASFSSVGAELEVMAPGAGVYSTYPTNTYATWNGTSMASPHVAGAAALILSKHPNLSASQVRNRLSSTATYLGSSFYYGKGLINVEAAAQ

SEQ ID NO: 21 - DNA, 824 bp, Artificial sequence
  Variant T58Q/L89S/L216W of Subtilisin Carlsberg
  gene (1)..(824): Variant T58Q/L89S/L216W of Subtilisin Carlsberg
  CDS (1)..(822)
gcgcaaaccgttccttacggcattcctctcattaaagcggacaaagtgcaggctcaaggctttaagggagcgaatgtaaaagtagccgtcctggatacaggaatccaagcttctcatccggacttgaacgtagtcggcggagcaagctttgtggctggcgaagcttataaccaggacggcaacggacacggcacacatgttgccggtacagtagctgcgcttgacaatacaacgggtgttttaggcgttgcgccaagcgtatcctcgtacgcggttaaagtactgaattcaagcggaagcggatcatacagcggcattgtaagcggaatcgagtgggcgacaacaaacggcatggatgttatcaatatgagccttgggggagcatcaggctcgacagcgatgaaacaggcagtcgacaatgcatatgcaagaggggtcgtcgttgtagctgcagcagggaacagcggatcttcaggaaacacgaatacaattggctatcctgcgaaatacgattctgtcatcgctgttggtgcggtagactctaacagcaacagagcttcattttccagcgtcggagcagagcttgaagtcatggctcctggcgcaggcgtatacagcacttacccaacgaacacttatgcaacatggaacggaacgtcaatggcttctcctcatgtagcgggagcagcagctttgatcttgtcaaaacatccgaacctttcagcttcacaagtccgcaaccgtctctccagcacggcgacttatttgggaagctccttctactatgggaaaggtctgatcaatgtcgaagctgccgctcaata

SEQ ID NO: 22 - protein, 274 aa, Artificial sequence
  Variant T58Q/L89S/L216W of Subtilisin Carlsberg
AQTVPYGIPLIKADKVQAQGFKGANVKVAVLDTGIQASHPDLNVVGGASFVAGEAYNQDGNGHGTHVAGTVAALDNTTGVLGVAPSVSSYAVKVLNSSGSGSYSGIVSGIEWATTNGMDVINMSLGGASGSTAMKQAVDNAYARGVVVVAAAGNSGSSGNTNTIGYPAKYDSVIAVGAVDSNSNRASFSSVGAELEVMAPGAGVYSTYPTNTYATWNGTSMASPHVAGAAALILSKHPNLSASQVRNRLSSTATYLGSSFYYGKGLINVEAAAQ

SEQ ID NO: 23 - DNA, 824 bp, Artificial sequence
  Variant L89S/L216W of Subtilisin Carlsberg
  gene (1)..(824): Variant L89S/L216W of Subtilisin Carlsberg
  CDS (1)..(822)
gcgcaaaccgttccttacggcattcctctcattaaagcggacaaagtgcaggctcaaggctttaagggagcgaatgtaaaagtagccgtcctggatacaggaatccaagcttctcatccggacttgaacgtagtcggcggagcaagctttgtggctggcgaagcttataacaccgacggcaacggacacggcacacatgttgccggtacagtagctgcgcttgacaatacaacgggtgttttaggcgttgcgccaagcgtatcctcgtacgcggttaaagtactgaattcaagcggaagcggatcatacagcggcattgtaagcggaatcgagtgggcgacaacaaacggcatggatgttatcaatatgagccttgggggagcatcaggctcgacagcgatgaaacaggcagtcgacaatgcatatgcaagaggggtcgtcgttgtagctgcagcagggaacagcggatcttcaggaaacacgaatacaattggctatcctgcgaaatacgattctgtcatcgctgttggtgcggtagactctaacagcaacagagcttcattttccagcgtcggagcagagcttgaagtcatggctcctggcgcaggcgtatacagcacttacccaacgaacacttatgcaacatggaacggaacgtcaatggcttctcctcatgtagcgggagcagcagctttgatcttgtcaaaacatccgaacctttcagcttcacaagtccgcaaccgtctctccagcacggcgacttatttgggaagctccttctactatgggaaaggtctgatcaatgtcgaagctgccgctcaata

SEQ ID NO: 24 - protein, 274 aa, Artificial sequence
  Variant L89S/L216W of Subtilisin Carlsberg
AQTVPYGIPLIKADKVQAQGFKGANVKVAVLDTGIQASHPDLNVVGGASFVAGEAYNTDGNGHGTHVAGTVAALDNTTGVLGVAPSVSSYAVKVLNSSGSGSYSGIVSGIEWATTNGMDVINMSLGGASGSTAMKQAVDNAYARGVVVVAAAGNSGSSGNTNTIGYPAKYDSVIAVGAVDSNSNRASFSSVGAELEVMAPGAGVYSTYPTNTYATWNGTSMASPHVAGAAALILSKHPNLSASQVRNRLSSTATYLGSSFYYGKGLINVEAAAQ

SEQ ID NO: 25 - DNA, 1140 bp, Bacillus licheniformis
  gene (1)..(1140): Alkaline protease (Subtilisin Carlsberg)
  mat_peptide (316)..()
  CDS (1)..(1137)
atgatgaggaaaaagagtttttggcttgggatgctgacggccttcatgctcgtgttcacgatggcattcagcgattccgcttctgctgctcaaccggcgaaaaatgttgaaaaggattatattgtcggatttaagtcaggagtgaaaaccgcatctgtcaaaaaggacatcatcaaagagagcggcggaaaagtggacaagcagtttagaatcatcaacgcggcaaaagcgaagctagacaaagaagcgcttaaggaagtcaaaaatgatccggatgtcgcttatgtggaagaggatcatgtggcccatgcgctagcgcaaaccgttccttacggcattcctctcattaaagcggacaaagtgcaggctcaaggctttaagggagcgaatgtaaaagtagccgtcctggatacaggaatccaagcttctcatccggacttgaacgtagtcggcggagcaagctttgtggctggcgaagcttataacaccgacggcaacggacacggcacacatgttgccggtacagtagctgcgcttgacaatacaacgggtgtattaggcgttgcgccaagcgtatccttgtacgcggttaaagtactgaattcaagcggaagcggatcatacagcggcattgtaagcggaatcgagtgggcgacaacaaacggcatggatgttatcaatatgagccttgggggagcatcaggctcgacagcgatgaaacaggcagtcgacaatgcatatgcaagaggggttgtcgttgtagctgcagcagggaacagcggatcttcaggaaacacgaatacaataggctatcctgcgaaatacgattctgtcatcgctgttggtgcggtagactctaacagcaacagagcttcattttccagcgtcggagcagagcttgaagtcatggctcctggcgcaggcgtatacagcacttacccaacgaacacttatgcaacattgaacggaacgtcaatggcttctcctcatgtagcgggagcagcagctttgatcttgtcaaaacatccgaacctttcagcttcacaagtccgcaaccgtctctccagcacggcgacttatttgggaagctccttctactatgggaaaggtctgatcaatgtcgaagctgccgctcaatga

SEQ ID NO: 26 - protein, 379 aa, Bacillus licheniformis
MMRKKSFWLGMLTAFMLVFTMAFSDSASAAQPAKNVEKDYIVGFKSGVKTASVKKDIIKESGGKVDKQFRIINAAKAKLDKEALKEVKNDPDVAYVEEDHVAHALAQTVPYGIPLIKADKVQAQGFKGANVKVAVLDTGIQASHPDLNVVGGASFVAGEAYNTDGNGHGTHVAGTVAALDNTTGVLGVAPSVSLYAVKVLNSSGSGSYSGIVSGIEWATTNGMDVINMSLGGASGSTAMKQAVDNAYARGVVVVAAAGNSGSSGNTNTIGYPAKYDSVIAVGAVDSNSNRASFSSVGAELEVMAPGAGVYSTYPTNTYATLNGTSMASPHVAGAAALILSKHPNLSASQVRNRLSSTATYLGSSFYYGKGLINVEAAAQ

SEQ ID NO: 27 - DNA, 1611 bp, Artificial sequence
  N-terminal deleted choline oxidase from Arthrobacter nicotianiae (DSMZ-ID 96-878)
  gene (1)..(1611): Choline oxidase (KC2s)
  misc_feature (1)..(3): First codon will be translated as Met or can artificially be changed into atg, coding for Met.
  CDS (1)..(1611)
ttgaacattgaaaagaaggacttcgactacattgtgatcggcggcggttctgccggcgctgcggtggcttctcgcctgagcgaggatccatcggtgagtgttgcactggttgaagcaggtcccgacgaccgcggctacgatgaagtgctgcagctggaccgctggatggaactgctggaatccggcttggactgggactacccgatcgaaccgcaggaaaacggcaactcgttcatgcgccatgcccgtgccaaggtcatgggcggctgctccagccacaactcctgcatcgccttctgggcgccgcgtgaggaccttgacgagtgggagtcgaagttcggcgccaccggctggaactccgagatggcctaccgcctgtacaagaagctggaaaccaatgaggacgccggaccggatgcaccgcaccacggcgactcgggcccggtcaagctgatgaacgtgccgccggtggacccctgcggcgtggcaatcctggacgccgccgagcaggcaggcatcccgcgcgccaagttcaacaacaacgagacggtcatcaacggcgcgaacttcttccagatcaaccgcctgccggacggcacccgtgcctcctcctcggtctcctacatccacccgatcgagggacgcgagaacttcttcctgctcaccggactgcaggcgcgcaagctgaacttcgacgccgacaagcgctgcaccggcgtggacgtggtggacggggccttcggccgcaccgtgacgctgaacgccgcccgcgaggtcgtcgtctccgccggcgccattgactcgccgaagctgctgatgctctccggcatcggacctgccgaacacctcaaggaggtcggcgtcgaggtgcttgtcgacgctccaggcgtgggcgaacacctgcaggaccacccggaaggcgtgatccagtgggaggctaagaagccgatggttgaaacctccacccagtggtgggaaatcggcatcttcgcaccgacccaggaaggcctcgaccgtccggatctgatgatgcactacggttcggtgcccttcgacatgcacaccctgcgttggggttacccgaccagcgagaacaccttctgcctgaccccgaatgtcacccatgcgaagtcgcgcggcaccgtgcgcctgcgcagctgcgacttcagcgacaagccgaaggtcgaccctcgctacttcaccgatccagagggccacgacgcccgggtcatgaccttcggcatccgcaaggcccgcgagatcgtcgcccagtcgccgatggcagagtgggctggcgaggagcagttcccgggcaaggacgtgcagactgacgaagagatcttcgactacctgcgccgctgccacaacactgtctaccacccggctggttcggtgcgcatgggcgccgaggacgatgtgatgagcccgctggatccgcagctgcgggtcaagggagtctcgggcctgcgcgtggccgacgcttcggtgatgccggagctggtcaccgtgaacccgaacatcacggtgatgatgatcggtgaacgctgcgccgagctgattcaggaaaaccctgtggaagcccgccagagcgaactggttagcgaagcctag

SEQ ID NO: 28 - protein, 536 aa, Artificial sequence
  N-terminal deleted choline oxidase from Arthrobacter nicotianiae (DSMZ-ID 96-878)
  misc_feature (1)..(3): First codon will be translated as Met or can artificially be changed into atg, coding for Met.
LNIEKKDFDYIVIGGGSAGAAVASRLSEDPSVSVALVEAGPDDRGYDEVLQLDRWMELLESGLDWDYPIEPQENGNSFMRHARAKVMGGCSSHNSCIAFWAPREDLDEWESKFGATGWNSEMAYRLYKKLETNEDAGPDAPHHGDSGPVKLMNVPPVDPCGVAILDAAEQAGIPRAKFNNNETVINGANFFQINRLPDGTRASSSVSYIHPIEGRENFFLLTGLQARKLNFDADKRCTGVDVVDGAFGRTVTLNAAREVVVSAGAIDSPKLLMLSGIGPAEHLKEVGVEVLVDAPGVGEHLQDHPEGVIQWEAKKPMVETSTQWWEIGIFAPTQEGLDRPDLMMHYGSVPFDMHTLRWGYPTSENTFCLTPNVTHAKSRGTVRLRSCDFSDKPKVDPRYFTDPEGHDARVMTFGIRKAREIVAQSPMAEWAGEEQFPGKDVQTDEEIFDYLRRCHNTVYHPAGSVRMGAEDDVMSPLDPQLRVKGVSGLRVADASVMPELVTVNPNITVMMIGERCAELIQENPVEARQSELVSEA